(19) United States
(12) Patent Application Publication
(10) Pub. No.: US 2022/0170910 A1
(43) Pub. Date: Jun. 2, 2022

(54) MULTIPLEXING REGULATORY ELEMENTS TO IDENTIFY CELL-TYPE SPECIFIC REGULATORY ELEMENTS

(71) Applicant: Encoded Therapeutics, Inc., South San Francisco, CA (US)

(72) Inventors: Kartik Ramamoorthi, South San Francisco, CA (US); Anne Tanenhaus, South San Francisco, CA (US); Sirika Wood, South San Francisco, CA (US); Martin Moorhead, South San Francisco, CA (US); Stephanie Tagliatela, South San Francisco, CA (US); Jerry S. Chen, South San Francisco, CA (US); Raghavendra Hosur, South San Francisco, CA (US)

(21) Appl. No.: 17/442,057

(22) PCT Filed: Mar. 20, 2020
(86) PCT No.: PCT/US2020/023881

§ 371 Date: Sep. 22, 2021

Related U.S. Application Data
(60) Provisional application No. 62/822,528, filed on Mar. 22, 2019.

(51) Int. Cl. G01N 33/50; C12Q 1/6876; C12N 15/86

(52) U.S. Cl. CPC G01N 33/5023; C12Q 1/6876; C12N 2750/14143; C12Q 2600/158; C12N 2830/008; C12N 15/86

(57) ABSTRACT

Provided herein is a high-throughput method for screening and identifying regulatory elements that provide selective expression in a particular cell type of interest. Also provided are nucleic acid compositions used in the high-throughput screening method.

The present application claims the benefit of priority to U.S. Provisional Patent Application No. 62/822,528, filed on Mar. 22, 2019, which is incorporated herein by reference.

### BACKGROUND OF THE DISCLOSURE

In recent years, the number of clinical trials which have utilized gene therapy for the treatment of disease has steadily increased. One of the major challenges these clinical trials have faced is the ability to control the level of expression or silencing of therapeutic genes in order to provide a balance between therapeutic efficacy and nonspecific toxicity due to overexpression of therapeutic protein or RNA interference-based sequences. Specifically, the level of transgene expression required to achieve a therapeutically relevant dose varies based on the inherent pathophysiology of the specific disease and on the nature of the transgene product (e.g., intracellular versus extracellular, structural versus enzymatic function). Additionally, cell-specific expression of the transgene is highly desirable as it provides the ability to selectively target pathologically relevant cell types (e.g., cancer cells) and reduces the likelihood of adverse events in patients. Thus, there is a need to identify regulatory elements and methods of use thereof for targeting gene therapy or gene expression to a tissue or cell type of interest, which can decrease off-target effects, increase therapeutic efficacy in the target tissue and/or cell type, and increase patient safety and tolerance by lowering the effective dose needed to achieve efficacy.

### SUMMARY OF THE DISCLOSURE

In some embodiments, the disclosure provides for a method of identifying a regulatory element that provides selective expression in a given cell type, comprising: a) providing cells with a mixture of vectors each comprising a candidate regulatory element operably linked to a transgene, wherein each vector further comprises a barcode; b) isolating RNA from a plurality of single cells expressing said transgene; c) identifying each of said single cells by sequencing the transcriptome of each of the single cells; and d) correlating the barcode in the transcriptome to a candidate regulatory element, thereby identifying a regulatory element that provides selective expression in the cell type. In some embodiments, the regulatory element selectively increases expression of the transgene in the cell type. In some embodiments, the regulatory element provides selective expression of the transgene that is at least 2 fold, at least 4 fold, at least 6 fold, at least 8 fold, or at least 10 fold greater or less as compared to expression driven by another candidate regulatory and/or a control regulatory element in the same cell type. In some embodiments, the regulatory element provides selective expression of the transgene that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less as compared to expression driven by another candidate regulatory element and/or control regulatory element in the same cell type. In some embodiments, the regulatory element provides selective expression of the transgene that is about 1.5 times, 2 times, 3 times, 4 times, 5 times, 6 times, 7 times, 7.5 times, 8 times, 9 times, or 10 times greater or less as compared to expression driven by another candidate regulatory element and/or control regulatory element in the same cell type. In some embodiments, the regulatory element provides selective expression of the transgene that is at least 2 fold, at least 4 fold, at least 6 fold, at least 8 fold, or at least 10 fold greater or less as compared to expression of the transgene from the same regulatory element in a different cell type. In some embodiments, the regulatory element provides selective expression of the transgene that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less compared to expression of the transgene from the same regulatory element in a different cell type. In some embodiments, the regulatory element provides selective expression of the transgene that is about 1.5 times, 2 times, 3 times, 4 times, 5 times, 6 times, 7 times, 7.5 times, 8 times, 9 times, or 10 times greater or less compared to expression of the transgene from the same regulatory element in a different cell type. In some embodiments, the regulatory element provides selective expression of the transgene in one cell type over at least one other cell type. In some embodiments, the regulatory element provides selective expression of the transgene in GABAergic neurons as compared to excitatory neurons. In other embodiments, the regulatory element provides selective expression of the transgene in GABAergic neuron subtypes such as GABAergic neurons that express glutamic acid decarboxylase 2 (GAD2), GAD1, NKX2.1, DLX1, DLX5, SST, PV or VIP. In other embodiments, the regulatory element provides selective expression of the transgene in parvalbumin (PV) neurons as compared to non-PV neurons. In some embodiments, the non-PV neuron is one or more of excitatory neurons, dopaminergic neurons, astrocytes, microglia, or motor neurons. In some embodiments, the regulatory element provides selective expression of the transgene that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less compared to expression of the transgene from the same regulatory element in a different GABAergic neuron subtypes. In some embodiments, the regulatory element provides selective expression of the transgene that is about 1.5 times, 2 times, 3 times, 4 times, 5 times, 6 times, 7 times, 7.5 times, 8 times, 9 times, or 10 times greater or less compared to expression of the transgene from the same regulatory element in a different GABAergic neuron subtypes.

In some embodiments, the disclosure provides for a method of identifying a regulatory element that provides selective expression of the transgene in a cell type or cellular subtype, comprising: a) providing cells with a mixture of vectors each comprising a candidate regulatory element operably linked to a transgene, wherein each vector further comprises a barcode; b) isolating RNA from a plurality of single cells expressing said transgene; c) identifying each of said single cells by sequencing the transcriptome of each of the single cells; d) correlating the barcode in the transcriptome to the candidate regulatory element; and e) comparing expression level of the transgene provided by each candidate regulatory element to a reference expression level of the transgene; thereby identifying the candidate regulatory element that provides selective expression of the transgene in the cell type. In some embodiments, the regulatory element selectively increases or decreases expression of the transgene in the cell type. In some embodiments, the reference expression level of the transgene is provided by a control regulatory element. In some embodiments, the regulatory element provides selective expression of the transgene that is at least 2 fold, at least 4 fold, at least 6 fold, at least 8 fold, or at least 10 fold greater or less as compared to expression driven by another candidate regulatory element and/or control regulatory element in the same cell type. In some embodiments, the regulatory element provides selective expression of the transgene that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less as compared to expression driven by another candidate regulatory element and/or control regulatory element in the same cell type. In some embodiments, the regulatory element provides selective expression of the transgene that is about 1.5 times, 2 times, 3 times, 4 times, 5 times, 6 times, 7 times, 7.5 times, 8 times, 9 times, or 10 times greater or less as compared to expression driven by another candidate regulatory element and/or control regulatory element in the same cell type. In some embodiments, the reference expression level of the transgene is provided by a pan-cellular regulatory element. In some embodiments, the pan-cellular regulatory element is selected from the group consisting of cytomegalovirus major immediate-early promoter (CMV), chicken β-actin promoter (CBA), CMV early enhancer/CBA promoter (CAG), elongation factor-1α promoter (EF1α), simian virus 40 promoter (SV40), phosphoglycerate kinase promoter (PGK), and the polyubiquitin C gene promoter (UBC). In some embodiments, the regulatory element provides selective expression of the transgene that is at least 2 fold, at least 4 fold, at least 6 fold, at least 8 fold, or at least 10 fold greater or less as compared to expression driven by a pan-cellular regulatory element in the same cell type. In some embodiments, the regulatory element provides selective expression of the transgene that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less as compared to expression driven by a pan-cellular regulatory element in the same cell type. In some embodiments, the regulatory element provides selective expression of the transgene that is about 1.5 times, 2 times, 3 times, 4 times, 5 times, 6 times, 7 times, 7.5 times, 8 times, 9 times, or 10 times greater or less as compared to expression driven by a pan-cellular regulatory element in the same cell type. In some embodiments, the regulatory element provides selective expression of the transgene in one cell type over at least one other cell type. In some embodiments, the regulatory element results in selective expression of the transgene in PV neurons as compared to non-PV neurons. In some embodiments, the non-PV neuron is one or more of excitatory neurons, dopaminergic neurons, astrocytes, microglia, or motor neurons.

In some embodiments, the disclosure provides for a method of identifying a cell type that selectively expresses a transgene operably linked to a regulatory element, comprising: a) providing cells with a mixture of vectors each comprising a candidate regulatory element operably linked to a transgene, wherein each vector further comprises a barcode; b) isolating RNA from a plurality of single cells expressing said transgene; c) identifying each of said single cells by sequencing the transcriptome of each of the single cells; d) correlating the barcode in the transcriptome to the candidate regulatory element; and e) comparing expression level of the transgene provided by the candidate regulatory element in one cell type to expression level of the same candidate regulatory element in a different cell type, thereby identifying the cell type that selectively expresses the transgene operably linked regulatory element. In some embodiments, the regulatory element selectively increases or decreases expression of the transgene in one cell type as compared to at least one other cell type. In some embodiments, the regulatory element provides selective expression of the transgene in one cell type that is at least 2 fold, at least 4 fold, at least 6 fold, at least 8 fold, or at least 10 fold greater or less as compared to expression driven by the regulatory element in at least one other cell type. In some embodiments, the regulatory element provides selective expression of the transgene in one cell type that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less as compared to expression driven by the regulatory element in at least one other cell type. In some embodiments, the regulatory element provides selective expression of the transgene in one cell type that is about 1.5 times, 2 times, 3 times, 4 times, 5 times, 6 times, 7 times, 7.5 times, 8 times, 9 times, or 10 times greater or less as compared to expression driven by the regulatory element in at least one other cell type. In some embodiments, the regulatory element results in selective expression of the transgene in PV neurons as compared to non-PV neurons. In some embodiments, the non-PV neuron is one or more of excitatory neurons, dopaminergic neurons, astrocytes, microglia, or motor neurons.

As can be readily appreciated, selectivity of expression driven by a regulatory element in a cell or cell type of interest can be measured in a number of ways. For example, selectivity of gene expression in a target cell type over non-target cell types can be measured by comparing the number of target cells that express a detectable level of a transcript from a gene that is operably linked to one or more regulatory elements to the total number of cells that express the gene. Such measurement, detection, and quantification can be done either in vivo or in vitro.

In some instances, selectivity for a specific cell type can be determined using a co-localization assay. In some cases, the co-localization assay is based on immunohistochemistry. In some cases, a detectable reporter gene is used as a transgene to allow the detection and/or measurement of gene expression in the cell type of interest. In some cases, a detectable marker, e.g., a fluorescent marker or an antibody, which specifically labels the target cell is used to detect and/or measure the target cells. In some cases, a co-localization assay employs imaging, e.g., fluorescent imaging, to determine the overlap between different fluorescent labels, e.g., overlap between a fluorescence signal indicative of a target cell and another fluorescence signal indicative of gene expression. In some cases, fluorescent labels used for a co-localization assay include a red fluorescent protein (RFP), such as a tdTomato reporter gene, and a green fluorescent reporter protein, such as eGFP.

In some embodiments, selectivity of a regulatory element in a cell type may be determined by an immunohistochemistry-based co-localization assay. In some embodiments, the assay comprises using: a) a detectable reporter gene as a transgene operably linked to regulatory element to measure transgene expression and, b) a binding agent that identifies a marker that is specific to a target cell type, wherein the binding agent is linked to a detectable label. In some embodiments, selectivity for a cell type can be determined or validated using an immunohistochemistry-based colocalization assay using: a) a transgene operably linked to regulatory element to measure transgene expression and, b) an antibody that identifies the cell type of interest linked to a second fluorescence label.

In some embodiments, the disclosure provides for a method of identifying a regulatory element that provides selective expression in a given cell type, comprising: a) providing cells with a mixture of vectors each comprising a candidate regulatory element operably linked to a transgene, wherein each vector further comprises a barcode; b) isolating RNA from a plurality of single cells expressing said transgene; c) identifying each of said single cells by sequencing the transcriptome of each of the single cells; and d) correlating the barcode in the transcriptome to a candidate regulatory element, thereby identifying a regulatory element that provides selective expression in the cell type. In order to increase detection of AAV constructs that fall below the threshold of detection in single nucleus RNAseq, an enrichment PCR step was performed prior to amplification. In some embodiments, the PCR enrichment step is performed prior to identifying each of said single cells by sequencing the transcriptome of each of the single cells. In some embodiments, the PCR enrichment step produces at least a 1-50 fold, at least a 2-25 fold, or at least a 3-10-fold amplification of a signal from an AAV construct.

In some embodiments of any of the methods disclosed herein, the RNA is selected from the group consisting of: mRNA, long noncoding RNA, antisense transcripts, and pri-miRNAs. In some embodiments of any of the methods disclosed herein, the vector is selected from the group consisting of: a plasmid, a viral vector, or a cosmid. In some embodiments of any of the methods disclosed herein, the viral vector is an adeno-associated virus (AAV) vector. In some embodiments of any of the methods disclosed herein, the AAV vector is AAV1, AAV8, AAV9, scAAV1, scAAV8, or scAAV9. In some embodiments of any of the methods disclosed herein, the AAV vector is AAV9. In some embodiments of any of the methods disclosed herein, the vector comprises a 5′ AAV inverted terminal repeat (ITR) sequence and a 3′ AAV ITR sequence. In some embodiments of any of the methods disclosed herein, the mixture of vectors comprises at least 10⁴ candidate regulatory elements. In some embodiments of any of the methods disclosed herein, each candidate regulatory element correlates to at least one unique barcode. In some embodiments of any of the methods disclosed herein, the transgene comprises a reporter gene sequence. In some embodiments of any of the methods disclosed herein, the reporter gene sequence is operably linked to a sequence encoding a nuclear binding domain. In some embodiments of any of the methods disclosed herein, the transgene comprises the barcode. In some embodiments of any of the methods disclosed herein, the reporter gene sequence comprises the barcode. In some embodiments of any of the methods disclosed herein, the barcode comprises alternative codons. In some embodiments of any of the methods disclosed herein, the sequence encoding the nuclear binding domain comprises the barcode. In some embodiments of any of the methods disclosed herein, the sequence encoding the nuclear binding domain encodes a Klarsicht/ANC-1/Syne homology (KASH) domain or a Sad1p/UNC-84 (SUN) domain protein, or biologically active fragment thereof. In some embodiments of any of the methods disclosed herein, the cell type belongs to a tissue selected from the group consisting of: connective tissue, muscular tissue, nervous tissue, and epithelial tissue.

In some embodiments, the disclosure provides for a nucleic acid molecule comprising a regulatory element operably linked to a transgene, wherein the nucleic acid molecule comprises a barcode. In some embodiments, the barcode comprises alternative codons. In some embodiments, the transgene comprises a reporter gene sequence. In some embodiments, the reporter gene sequence is operably linked to a nucleotide sequence encoding a nuclear binding domain sequence. In some embodiments, the nuclear binding domain sequence encodes a KASH domain or SUN domain protein or biologically active fragment thereof. In some embodiments, the regulatory element is non-naturally occurring. In some embodiments, the reporter gene sequence encodes a fluorescent protein. In some embodiments, the fluorescent protein is a green fluorescent protein (GFP), enhanced green fluorescent protein (EGFP), a yellow fluorescent protein (YFP), such as mBanana, a red fluorescent protein (RFP), such as mCherry, DsRed, dTomato, tdTomato, mHoneydew, or mStrawberry, TagRFP, far-red fluorescent pamidronate (FRFP), such as mGrape1 or mGrape2, a cyan fluorescent protein (CFP), a blue fluorescent protein (BFP), enhanced cyan fluorescent protein (ECFP), ultramarine fluorescent protein (UMFP), orange fluorescent protein (OFP), such as mOrange or mTangerine, red (orange) fluorescent protein (mROFP), TagCFP, or a tetracystein fluorescent motif. In some embodiments, the transgene comprises the barcode. In some embodiments, the sequence encoding the nuclear binding domain comprises the barcode. In some embodiments, the reporter gene sequence comprises the barcode. In some embodiments, the barcode is placed within a coding region of the transgene. In some embodiments, the nucleic acid molecule comprises a non-coding region, and wherein the barcode is placed within a non-coding region of the transgene. In some embodiments, the nucleic acid molecule comprises an untranslated region (UTR) and the barcode is placed within the UTR. In some embodiments, the barcode sequence is located within about 25, 30, 35, 50, 100, 150, 200, 250, 300, 350, 400, 450 or 500 bases from the start of the polyA tail in the nucleic acid. In other embodiments, the nucleic acid comprises a polyA sequence, and wherein the barcode is placed at least 35 bases upstream of the polyA sequence. In some embodiments, the barcode is placed upstream of the transcription start site.

In some embodiments, the disclosure provides for a nucleic acid molecule, wherein the nucleic acid molecule is an RNA molecule transcribed from a DNA molecule, wherein the RNA molecule comprises a transgene and a barcode sequence, wherein the DNA molecule comprises a regulatory element, and wherein the barcode sequence in the RNA molecule correlates with the regulatory element in the DNA molecule. In some embodiments, the transgene comprises a reporter gene sequence. In some embodiments, the reporter gene sequence is operably linked to a nucleotide sequence encoding a nuclear binding domain. In some embodiments, the nuclear binding domain is a KASH domain or SUN domain protein or biologically active fragment thereof. In some embodiments, the regulatory element is non-naturally occurring. In some embodiments, the reporter gene sequence encodes a fluorescent protein. In some embodiments, the fluorescent protein is a green fluorescent protein (GFP), enhanced green fluorescent protein (EGFP), a yellow fluorescent protein (YFP), such as mBanana, a red fluorescent protein (RFP), such as mCherry, DsRed, dTomato, tdTomato, mHoneydew, or mStrawberry, TagRFP, far-red fluorescent pamidronate (FRFP), such as mGrape1 or mGrape2, a cyan fluorescent protein (CFP), a blue fluorescent protein (BFP), enhanced cyan fluorescent protein (ECFP), ultramarine fluorescent protein (UMFP), orange fluorescent protein (OFP), such as mOrange or mTangerine, red (orange) fluorescent protein (mROFP), TagCFP, or a tetracystein fluorescent motif. In some embodiments, the transgene comprises the barcode. In some embodiments, the sequence encoding the nuclear binding domain comprises the barcode. In some embodiments, the reporter gene sequence comprises the barcode. In some embodiments, the barcode comprises alternative codons. In some embodiments, the nucleic acid molecule comprises an untranslated region (UTR) and the barcode is placed within the UTR. In some embodiments, the nucleic acid molecule comprises a polyA sequence, and wherein the barcode is placed at least 30 to 50 bases upstream of the polyA sequence. In some embodiments, the nucleic acid molecule is connected to a microparticle. In some embodiments, the microparticle is a bead. In some embodiments, the microparticle is connected to a microparticle polynucleotide molecule. In some embodiments, the nucleic acid molecule is connected to the microparticle via the microparticle polynucleotide molecule. In some embodiments, the microparticle polynucleotide molecule comprises a primer sequence. In some embodiments, the microparticle polynucleotide molecule comprises a cell barcode sequence. In some embodiments, the microparticle polynucleotide molecule comprises a Unique Molecular Identifier (UMI) nucleotide sequence. In some embodiments, the microparticle polynucleotide molecule comprises an oligo-dT sequence. In some embodiments, the microparticle polynucleotide molecule comprises: a) a primer sequence, b) a cell barcode sequence, c) a Unique Molecular Identifier (UMI) nucleotide sequence, and d) an oligo-dT sequence; wherein the nucleic acid comprises a polyA nucleotide sequence, and wherein the microparticle is connected to a)-d) in the following order: microparticle--a)--b)--c)--d); and wherein the polyA nucleotide sequence is hybridized with the oligo-dT sequence. In some embodiments, the microparticle is a bead.

In some embodiments, the disclosure provides for a vector comprising any of the the nucleic acids disclose herein. In some embodiments, the vector is a viral vector. In some embodiments, the vector is an adeno-associated viral vector. In some embodiments, the adeno-associated viral vector is any one of AAV1, AAV2, AAV3, AAV4, AAV5, AAV6, AAV7, AAV8, AAV9, AAV10, AAV11, AAV12, rh10, and hybrids thereof, avian AAV, bovine AAV, canine AAV, equine AAV, primate AAV, non-primate AAV, or ovine AAV. In some embodiments, the adeno-associated viral vector is an AAV9 vector.

In some embodiments, the disclosure provides for a cell comprising any of the nucleic acids disclosed herein.

In some embodiments, the disclosure provides for a cell comprising any of the vectors disclosed herein.

In some embodiments, the disclosure provides for a microparticle connected to one or more of any of the nucleic acids disclosed herein. In some embodiments, the microparticle is a bead. In some embodiments, the microparticle is connected to a microparticle polynucleotide molecule. In some embodiments, the microparticle polynucleotide molecule comprises a primer sequence. In some embodiments, the microparticle polynucleotide molecule comprises a Unique Molecular Identifier (UMI). In some embodiments, the microparticle polynucleotide molecule comprises an oligo-dT sequence. In some embodiments, the nucleic acid comprises a polyA nucleotide sequence, and wherein the polyA nucleotide sequence is hybridized to the oligo-dT sequence. In some embodiments, the microparticle polynucleotide molecule comprises: a) a primer sequence, b) a cell barcode sequence, c) a Unique Molecular Identifier (UMI) sequence, and d) an oligo-dT sequence; wherein the nucleic acid comprises a polyA nucleotide sequence, wherein the microparticle is connected to a)-d) in the following order: microparticle--a)--b)--c)--d); and wherein the polyA nucleotide sequence is hybridized with the oligo-dT sequence. In some embodiments, the microparticle is a bead.

In some embodiments, the disclosure provides for a droplet comprising any of the nucleic acid molecules disclosed herein.

In some embodiments, the disclosure provides for a droplet comprising any of the cells disclosed herein.

In some embodiments, the disclosure provides for a droplet comprising any of the microparticles disclosed herein.

In some embodiments, the disclosure provides for a droplet comprising any of the cells disclosed herein and any of the microparticles disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a simplified illustration of a method for multiplexing regulatory elements (“REs”) in vivo to evaluate RE specificity by using single nucleus RNAseq. FIG. 1B is a simplified schematic of the workflow of the 10× Genomics Chromium Single Cell 3′ v2 kit for single nucleus RNAseq.

FIG. 2 illustrates the clusters which are annotated based on literature-derived canonical biomarkers. The biomarkers are defined in Table 2. “Exc”=Excitatory neurons; “GABA”=GABAergic neurons; “NonN”=Non-neuronal cells; “TPM”=transcripts per million.

FIG. 3 illustrates the expression of each barcoded AAV transgene under either a CamKII promoter, CBA promoter, or a RE1regulatory element (e.g., SEQ ID NO: 1) within each cell population. “Exc”=Excitatory neurons; “GABA”=GABAergic neurons; “NonN”=Non-neuronal cells; “TPM”=transcripts per million.

FIG. 4 illustrates the CBA-Normalized Fold Change for each AAV transgene within each cell population (i.e., expression of each AAV transgene relative to the average CBA expression within a given cell population). Fold-changes within the excitatory population are normalized to 1. Each barcoded AAV transgene is shown separately. “Exc”=Excitatory neurons; “GABA”=GABAergic neurons; “NonN”=Non-neuronal cells.

FIG. 5 illustrates the CBA-Normalized Fold Change for each AAV transgene within each cell population. Expression values were averaged between the two barcoded versions of each AAV transgene. Fold-changes within the excitatory population are normalized to 1. “Exc”=Excitatory neurons; “GABA”=GABAergic neurons; “NonN”=Non-neuronal cells.

FIG. 6 illustrates AAV transgene expression in excitatory cells compared with four GABA sub-populations (sub-populations positive for PV (parvalbumin), VIP (vasoactive intestinal polypeptide), Sst (somatostatin), or Ndnf-Reln (Neuron-Derived Neurotrophic Factor-Reelin)).

FIG. 7 is a graph showing expression (TPM) of the AAV L3 library for each regulatory elements in GABAergic and excitatory neurons. Control regulatory element are: CBA (Construct 1), EF1α (Construct 2), and RE1 (Construct 3).

FIG. 8 is a graph showing expression (TPM) of the AAV L3.2 library for each regulatory elements in GABAergic and excitatory neurons. Control regulatory element are: CBA (Construct 1), EF1α (Construct 2), and RE1 (Construct 3).

FIG. 9 is a graph showing cell type specific expression of various REs in GABAergic neurons (AAV L3 and AAV L3.2 libraries). Expression for each construct was normalized to the average TPM expression of the AAV EF1α associated transgene. Control regulatory elements are: CBA (Construct 1), EF1α (Construct 2), and RE1 (Construct 3).

FIG. 11 is a graph showing cell type specific expression (AAV9 L3.2 library) within specific cell types within the class of GABAergic neurons (e.g., PV, SST, and VIP cells). Expression for each construct was normalized to the average TPM expression of the AAV EF1α associated transgene. Control regulatory elements are: CBA (Construct 1), EF1α(Construct 2), and RE1 (Construct 3).

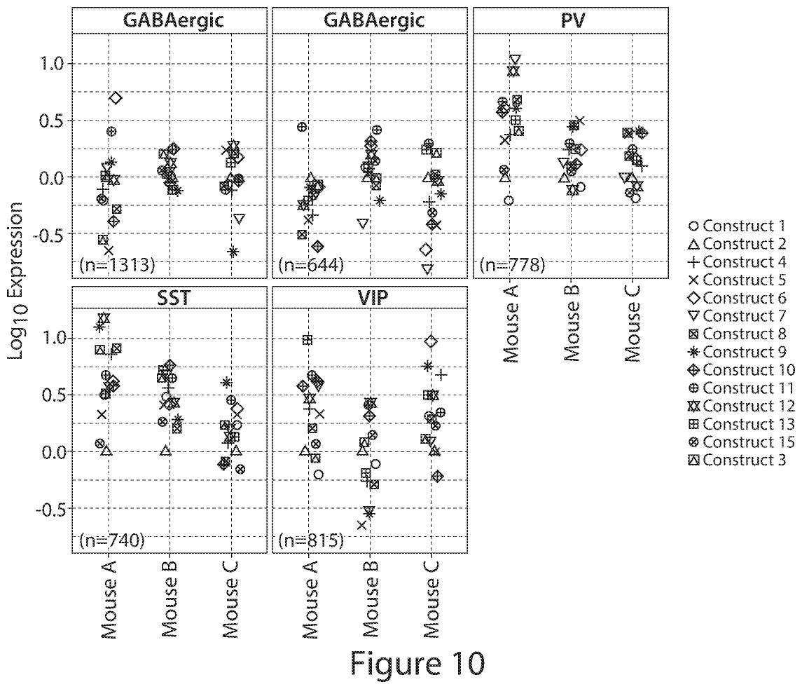

### DETAILED DESCRIPTION OF THE DISCLOSURE

One challenge in gene therapy is ensuring that the transgene of interest is expressed in an appropriate cell type of interest, or the target cell type, to effect or target gene expression without or with minimal off-target effects. Traditional methods for targeted gene therapy have often relied on delivery methods and/or vehicles (e.g., varying the viruses used or capsid sequences of viruses). Therapeutic methods involving the delivery of a transgene also have a number of challenges, such as limitations in the size of the transgene, as many vectors have a limited capacity for transgene size. For instance, AAV vectors have a maximum capacity of approximately 4.7 kb, and the two inverted terminal repeats (ITRs) are about 0.2-0.3 kb total, leaving approximately 4.4 kb that needs to accommodate both the transgene and the regulatory elements that control expression of the transgene.

The present disclosure provides compositions and methods of screening regulatory elements to identify a regulatory element that provides selective expression of a gene of interest (a transgene) in a cell type of interest. In particular, the present disclosure provides methods of screening numerous (e.g., 10 to 10⁴) regulatory elements (e.g., in vivo or in vitro) in order to identify regulatory elements that achieve a physiologically or therapeutically relevant level of expression of a transgene in a specific population of cells. In some embodiments, the present disclosure provides a high-throughput system for identifying a regulatory element, among thousands of candidate regulatory elements, that provides selective expression of a transgene of interest in a cell type of interest (thereby effectively minimizing or eliminating off-target effects when used to drive expression of a transgene in a therapeutic setting). The present disclosure can also be used to identify which cell type is better suited (or more selective) for expressing a transgene using a regulatory element of interest. That is, using the present methods, a given regulatory element can be “matched” to a given cell type (e.g., PV neurons, cardiomyocytes, etc.) for optimal selective expression of any transgene of interest. By identifying regulatory elements using the methods disclosed herein, it is possible to improve the efficacy of a gene therapy, decrease the effective dose needed to result in a therapeutic effect, minimize adverse effects or off-target effect, and/or increase patient safety and/or tolerance. The present disclosure also provides compositions useful for practicing the present methods.

### Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “AAV” is an abbreviation for adeno-associated virus and may be used to refer to the virus itself or a derivative thereof. The term covers all serotypes, subtypes, and both naturally occurring and recombinant forms, except where required otherwise. The abbreviation “rAAV” refers to recombinant adeno-associated virus. The term “AAV” includes AAV1, AAV2, AAV3, AAV4, AAV5, AAV6, AAV7, AAV8, AAV9, AAV10, AAV 11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “rAAV vector” as used herein refers to an AAV vector comprising a polynucleotide sequence not of AAV origin (i.e., a polynucleotide heterologous to AAV), typically a sequence of interest for the genetic transformation of a cell. In some embodiments, the heterologous polynucleotide is flanked by at least one, and generally by two, AAV inverted terminal repeat sequences (ITRs). An rAAV vector may either be single-stranded (ssAAV) or self-complementary (scAAV). An “AAV virus” or “AAV viral particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV viral particle” or simply an “rAAV particle”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% above or below a given value.

The term “connected to” or “connect to” means an association between two or more entities, e.g., an association between two or more of any of the nucleic acid disclosed herein. Two entities may be connected to each other by, for example, a covalent bond (e.g., a phosphodiester bond connecting two or more nucleic acid nucleotide chains together) or hydrogen bonds (e.g., the hydrogen bonds associated with hybridization between a nucleotide sequence on one nucleic acid molecule and the complementary nucleotide sequence on another nucleic acid molecule).

It is understood that wherever embodiments are described herein with the language “comprising,” otherwise analogous embodiments described in terms of “consisting of” and/or “consisting essentially of” are also provided.

The terms “determining”, “measuring”, “evaluating”, “assessing”, “assaying”, “analyzing”, and their grammatical equivalents can be used interchangeably herein to refer to any form of measurement and include determining if an element is present or not (for example, detection). These terms can include both quantitative and/or qualitative determinations. Assessing may be relative or absolute.

The term “expression” or “expressing” refers to the process by which a nucleic acid sequence or nucleic acid molecule and/or a polynucleotide is transcribed from a DNA template (such as into mRNA or other RNA transcript) and/or the process by which a transcribed mRNA is subsequently translated into peptides, polypeptides, or proteins. The term “expression” or “expressing” also may refer to the transcription of a non-coding RNA molecule, such as an antisense RNA molecule, an RNAi molecule and/or a short hairpin RNA molecule. Transcripts and encoded polypeptides may be collectively referred to as “gene product.” If the polynucleotide is derived from genomic DNA, expression may include splicing of the mRNA in a eukaryotic cell.

A “fragment” of a nucleotide or peptide sequence is meant to refer to a sequence that is less than that believed to be the “full-length” sequence.

A “functional fragment” of a DNA or protein sequence refers to a biologically active fragment of the sequence that is shorter than the full-length or reference DNA or protein sequence, but which retains at least one biological activity (either functional or structural) that is substantially similar to a biological activity of the full-length or reference DNA or protein sequence.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

The term “in vivo” refers to an event that takes place in a subject's body.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally, at a chromosomal location that is different from its natural chromosomal location, or contains only coding sequences.

As used herein, “operably linked”, “operable linkage”, “operatively linked”, or grammatical equivalents thereof refer to juxtaposition of genetic elements, e.g., a promoter, an enhancer, a polyadenylation sequence, etc., wherein the elements are in a relationship permitting them to operate in the expected manner. For instance, a regulatory element comprising a promoter is operatively linked to a coding region if the regulatory element helps initiate transcription of the coding sequence. In some embodiments, there may be intervening residues between the regulatory element and coding region so long as this functional relationship is maintained.

The term “regulatory element” (used interchangeably with “RE”) refers to a nucleic acid sequence or genetic element which is capable of influencing (e.g., increasing, decreasing, or modulating) expression of an operably linked sequence, such as a gene. Regulatory elements include, but are not limited to, a promoter, an enhancer, a repressor, a silencer, an insulator sequence, an intron, a UTR, an inverted terminal repeat (ITR) sequence, a long terminal repeat sequence (LTR), a stability element, a micro RNA binding site, a posttranslational response element, or a polyA sequence, or a combination thereof. Regulatory elements can function at the DNA and/or the RNA level, e.g., by modulating gene expression at the transcriptional phase, post-transcriptional phase, or translational phase of gene expression; by modulating the level of translation (e.g., stability elements that stabilize mRNA for translation), RNA cleavage, RNA splicing, and/or transcriptional termination; by recruiting transcriptional factors to a coding region that increase gene expression; by increasing the rate at which RNA transcripts are produced, increasing the stability of RNA produced, and/or increasing the rate of protein synthesis from RNA transcripts; and/or by preventing RNA degradation and/or increasing its stability to facilitate protein synthesis. In some embodiments, a regulatory element refers to an enhancer, repressor, promoter, or a combination thereof, particularly an enhancer plus promoter combination or a repressor plus promoter combination. In some embodiments, the regulatory element is derived from a human sequence, e.g., the sequence is at least 70%, at least 75%, at least 80%, at least 85%, at least 90%, at least 93%, at least 95%, at least 96%, at least 97%, at least 98%, or at least 99% sequence identity to a sequence derived from a human sequence. In some embodiments, the regulatory element is a synthetic sequence.

A “candidate regulatory element” means a regulatory element that is to be assessed in any of the assay methods of the present disclosure. A “candidate regulatory element” can include one regulatory element or a combination of more than one regulatory elements.

A “control regulatory element” means a regulatory element to which a candidate regulatory element is compared. In some embodiments, a “control regulatory element” is a regulatory element with a well-characterized expression profile. For example, in some embodiments, a “control regulatory element” is a naturally occurring regulatory element, such as the chicken β-actin promoter (CBA).

As used herein “RNAseq” or “RNA-seq” is used to refer to a transcriptomic approach where the total complement of RNAs from a given sample is isolated and sequenced using high-throughput next generation sequencing (NGS) technologies (e.g., SOLiD, 454, Illumina, or ION Torrent). In some embodiments, RNAseq transcripts are reverse-transcribed into cDNA, and adapters are ligated to each end of the cDNA. In some embodiments, sequencing can be done either unidirectional (single-end sequencing) or bidirectional (paired-end sequencing) and then aligned to a reference genome database.

In general, “sequence identity” or “sequence homology”, which can be used interchangeably, refer to an exact nucleotide-to-nucleotide or amino acid-to-amino acid correspondence of two polynucleotides or polypeptide sequences, respectively. Two or more sequences (polynucleotide or amino acid) can be compared by determining their “percent identity”, also referred to as “percent homology.” The percent identity to a reference sequence (e.g., nucleic acid or amino acid sequence) may be calculated as the number of exact matches between two optimally aligned sequences divided by the length of the reference sequence and multiplied by 100. Conservative substitutions are not considered as matches when determining the number of matches for sequence identity. It will be appreciated that where the length of a first sequence (A) is not equal to the length of a second sequence (B), the percent identity of A:B sequence will be different than the percent identity of B:A sequence. Sequence alignments, such as for the purpose of assessing percent identity, may be performed by any suitable alignment algorithm or program, including but not limited to the Needleman-Wunsch algorithm (see, e.g., the EMBOSS Needle aligner available on the world wide web at ebi.ac.uk/Tools/psa/emboss_needle/), the BLAST algorithm (see, e.g., the BLAST alignment tool available on the world wide web at blast.ncbi.nlm.nih.gov/Blast.cgi), the Smith-Waterman algorithm (see, e.g., the EMBOSS Water aligner available on the world wide web at ebi.ac.uk/Tools/psa/emboss_water/), and Clustal Omega alignment program (see e.g., the world wide web at clustal.org/omega/ and F. Sievers et al., Mol Sys Biol. 7: 539 (2011)). Optimal alignment may be assessed using any suitable parameters of a chosen algorithm, including default parameters. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997).

The terms “subject” and “individual” are used interchangeably herein to refer to a vertebrate, preferably a mammal, more preferably a human.

A “variant” of a nucleotide sequence refers to a sequence having a genetic alteration or a mutation as compared to the most common wild-type DNA sequence (e.g., cDNA or a sequence referenced by its GenBank accession number) or a specified reference sequence.

A “vector” as used herein refers to a nucleic acid molecule that can be used to mediate delivery of another nucleic acid molecule to which it is linked into a cell where it can be replicated or expressed. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Other examples of vectors include plasmids, viral vectors, and cosmids.

The term “transgene” as used herein refers to polynucleotide sequences not naturally present in a particular cell, polynucleotide sequences exogenously added to a cell, and/or heterologous polynucleotide sequences contained in a vector (e.g., a viral vector such as an AAV vector). Transgenes can comprise natural sequences (e.g., sequence encoding a natural protein) as well as synthetic sequences. A transgene can comprise coding and/or non-coding sequences. In some embodiments, a transgene is a sequence operably linked to a regulatory element.

The term “selective expression” or “selectively expresses” refers to a selective increase or decrease in expression of a transgene relative to a reference expression level (as defined herein) as driven by a regulatory element (e.g., a candidate regulatory element) to which the transgene is operably linked. In various embodiments, selective expression of a transgene provided by a regulatory element includes: transgene expression in one cell type that is higher or lower than the level of transgene expression provided by a different regulatory element in the same cell type; transgene expression in one cell type that is higher or lower than the level of transgene expression provided by the same regulatory element in one or more other cell type(s); an increase or decrease in transgene expression in a particular cell type that is not observed in a different cell type (a reference cell type) expressing the same transgene operably linked to the same regulatory element; an increase or decrease in the ratio of the number of target cells of one particular cell type expressing a transgene operably linked to a candidate regulatory element in a population of cells (e.g., of a target tissue) as compared to the total number of cells in the population expressing the transgene operably linked to the same regulatory element; an increase or decrease in the ratio of the number of target cells expressing a transgene vs. the total number of cells expressing the transgene when the transgene is operably linked to a candidate regulatory element as compared to the ratio obtained when the transgene is operably linked to a different regulatory element; transgene expression in a target cell at a level that is at least 10%, 20%, 30%, 40%, 50%, 60%, 70%, 80%, 90%, 100%, 120%, 140%, 150%, 200%, 250%, 300%, 350%, 400%, 450%, 500% greater than the transgene expression level in non-target cells or non-target tissues (e.g., in a human subject); transgene expression that occurs at meaningful (e.g., therapeutically relevant) levels in at least a portion of the cell type of interest in a target target tissue; and/or transgene expression that occurs primarily in the cells of a target tissue versus those of other tissues.

The term “reference expression level” refers to a level of expression provided by: another candidate regulatory element in the same cell type of interest; the same candidate regulatory in a different cell type; a known, control regulatory element in the same cell type of interest; and/or a known, control regulatory element in a different cell type.

The term “pan-cellular” in the context of a regulatory element refers to a regulatory element that drives expression of a gene or transgene to which it is operably linked across many cell types (or ubiquitiously). Some examples of such regulatory elements include cytomegalovirus major immediate-early promoter (CMV), chicken β-actin promoter (CBA), CMV early enhancer/CBA promoter (CAG), elongation factor-1α promoter (EF1α), simian virus 40 promoter (SV40), phosphoglycerate kinase promoter (PGK), and the polyubiquitin C gene promoter (UBC).

The term “cell type” refers to a distinct morphological or functional form of a cell. A cell type may be identified using various characteristics, including, for example: gene expression profile, epigenetic profile, non-coding RNA profile, protein expression profile, cell surface markers, differentiation potential, proliferative capacity, response to stimuli or signals, anatomical location, morphology, staining profiles, and/or timing of appearance during development, and/or any combination of the foregoing. In some embodiments, a cell type is defined based on a specific characteristic or combination of characteristics. For example, in some embodiments, a cell type is defined based on the expression of a specific gene or combination of genes. In some embodiments, a cell type can be defined by the tissue from which it was sourced or originated, e.g., connective tissue, muscular tissue, nervous tissue, or epithelial tissue. By way of example, cells derived from muscular tissue include cardiac muscle cells (e.g., cardiomyocytes), smooth muscle cells, skeletal muscle cells and various subpopulations of any of the foregoing. A variety of different cell types can be obtained from a single organism (or from the same species of organism), a single organ, or a single tissue. Exemplary cell types include, but are not limited to, urinary bladder, pancreatic epithelial, pancreatic alpha, pancreatic beta, pancreatic endothelial, bone marrow lymphoblast, bone marrow B lymphoblast, bone marrow macrophage, bone marrow erythroblast, bone marrow dendritic, bone marrow adipocyte, bone marrow osteocyte, bone marrow chondrocyte, promyeloblast, bone marrow megakaryoblast, bladder, brain B lymphocyte, brain glial, neuron, brain astrocyte, neuroectoderm, brain macrophage, brain microglia, brain epithelial, cardiomyocyte, cortical neuron, brain fibroblast, breast epithelial, colon epithelial, colon B lymphocyte, mammary epithelial, mammary myoepithelial, mammary fibroblast, colon enterocyte, cervix epithelial, ovary epithelial, ovary fibroblast, breast duct epithelial, tongue epithelial, tonsil dendritic, tonsil B lymphocyte, peripheral blood lymphoblast, peripheral blood T lymphoblast, peripheral blood cutaneous T lymphocyte, peripheral blood natural killer, peripheral blood B lymphoblast, peripheral blood monocyte, peripheral blood myeloblast, peripheral blood monoblast, peripheral blood promyeloblast, peripheral blood macrophage, peripheral blood basophil, liver endothelial, liver mast, liver epithelial, liver B lymphocyte, spleen endothelial, spleen epithelial, spleen B lymphocyte, liver hepatocyte, liver Alexander, liver fibroblast, lung epithelial, bronchus epithelial, lung fibroblast, lung B lymphocyte, lung Schwann, lung squamous, lung macrophage, lung osteoblast, neuroendocrine, lung alveolar, stomach epithelial, and stomach fibroblast.

The term “reporter molecule” refers to a molecule (e.g., a protein) that can be used as an indicator of the occurrence or level of a particular biological process, activity, event, or state in a cell or organism. Reporter molecules typically have one or more properties or enzymatic activities that allow them to be readily measured or that allow selection of a cell that expresses the reporter molecule. In general, a cell can be assayed for the presence of a reporter molecule by determining the presence and/or measuring the level of the reporter molecule itself (e.g., DNA, RNA and/or protein) or an enzymatic activity of the reporter molecule. Detectable characteristics or activities that a reporter molecule may have include, e.g., fluorescence, bioluminescence, ability to bind to specific substrates, sequence, ability to catalyze a reaction that produces a fluorescent or colored substance in the presence of a suitable substrate, or other readouts based on emission and/or absorption of photons (light). Typically, a reporter molecule is a molecule that is not endogenously expressed by a cell or organism in which the reporter molecule is used, or a molecule that has been modified to allow selective detection over an endogenous molecule.

The term “domain” or “protein domain” refers to a part of a protein chain that may exist and function independently of the rest of the protein chain.

The term “non-natural” or “non-naturally” or “variant” should be taken to mean the exhibition of qualities that deviate from what occurs in nature.

The term “statistically significant” or “significantly” refers to statistical significance and generally means at least two standard deviation (2SD) away from a reference level. It is defined as the probability of making a decision to reject the null hypothesis when the null hypothesis is actually true.

The terms “decrease”, “reduced”, “reduction”, “decrease” or “inhibit” are all used herein generally to mean an observable decrease in a measured parameter.

The terms “increased”, “increase” or “enhance” or “activate” are all used herein to generally mean an observable increase in a measured parameter.

As used herein, the terms “treat”, “treatment”, “therapy” and the like refer to obtaining a desired pharmacologic and/or physiologic effect, including, but not limited to, alleviating, delaying or slowing progression, reducing effects or symptoms, preventing onset, preventing reoccurrence, inhibiting, ameliorating onset of a diseases or disorder, obtaining a beneficial or desired result with respect to a disease, disorder, or medical condition, such as a therapeutic benefit and/or a prophylactic benefit. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease, or gradations of any of the following. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some embodiments, for prophylactic benefit, the compositions are administered to a subject at risk of developing a particular disease, or to a subject reporting one or more of the physiological symptoms of a disease, even though a diagnosis of this disease may not have been made. The methods of the present disclosure may be used with any mammal. In some embodiments, the treatment can result in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

Unless otherwise indicated, all terms used herein have the same meaning as they would to one skilled in the art and the practice of the present invention will employ, conventional techniques of molecular biology, microbiology, and recombinant DNA technology, which are within the knowledge of those of skill of the art.

### Nucleic Acid Compositions

In some embodiments, the present disclosure relates to methods of screening numerous (e.g., 10 to 10⁴) candidate regulatory elements (e.g., in vivo or in vitro) in order to identify regulatory elements that provide selective expression of a transgene of interest in a specific population of cells. In some embodiments, the present disclosure relates to methods of screening 10 to 20, 10 to 50, 10 to 100, 10 to 200, 10 to 400, 10 to 600, 10 to 800, 10 to 1000, 10 to 3000, 10 to 6000, 10 to 10,000, 10 to 13,000, 10 to 16,000, 10 to 20,000, 10 to 30,000, 10 to 40,000, 10 to 50,000, 10 to 60,000, 10 to 70,000, 10 to 80,000, 10 to 90,000, 10 to 100,000, 10 to 500,000, or 10 to 1,000,000 candidate regulatory elements (e.g., in vivo or in vitro) in order to identify regulatory elements that provide selective expression of a transgene of interest in a specific population of cells. The methods include providing cells (e.g., a population of cells or tissue) with a mixture of vectors each comprising a nucleic acid molecule having one or more candidate regulatory elements operably linked to a sequence encoding a transgene (e.g., comprising a reporter gene) and a barcode sequence for regulatory element identification. Thus, in some aspects, provided herein are nucleic acid components and compositions useful for practicing the methods of the present disclosure.

In some embodiments, the nucleic acid is a DNA molecule. In some embodiments, the nucleic acid is an RNA molecule. In some embodiments, the nucleic acid is a DNA molecule in any of the vectors disclosed herein. In some embodiments, the nucleic acid molecule comprises any of the transgenes disclosed herein. In some embodiments, the nucleic acid molecule comprises any of the candidate regulatory elements disclosed herein. In some embodiments, the nucleic acid comprises any of the barcode sequences disclosed herein. In some embodiments, the nucleic acid is a DNA molecule comprising any of the transgenes disclosed herein, any of the candidate regulatory elements disclosed herein, and any of the barcode sequences disclosed herein. In some embodiments, the nucleic acid molecule is an RNA nucleic acid molecule comprising any of the transgenes disclosed herein and any of the barcode sequences disclosed herein. In some embodiments, the RNA molecule is transcribed from any of the DNA molecules disclosed herein (e.g., a DNA molecule comprising any of the transgenes, candidate regulatory elements, and barcode sequences disclosed herein). In some embodiments, the RNA molecule is transcribed from any of the DNA molecules disclosed herein (e.g., a DNA molecule comprising any of the transgenes, candidate regulatory elements, and barcode sequences disclosed herein), wherein the RNA molecule comprises a transgene and a barcode sequence, wherein the barcode sequence in the RNA molecule correlates with the candidate regulatory element in the DNA molecule.

As discussed in greater detail below, in some embodiments, any of the nucleic acid molecules disclosed herein is connected to a microparticle. In particular embodiments, the nucleic acid molecule that is connected to the microparticle is an RNA molecule transcribed from a DNA molecule (e.g., any of the DNA molecules disclosed herein). In some embodiments, the RNA molecule comprises a transgene and a barcode sequence. In some embodiments, the DNA molecule comprises a regulatory element, wherein the barcode sequence in the RNA molecule correlates with the regulatory element in the DNA molecule. In some embodiments, the microparticle is a bead. In some embodiments, the microparticle is connected to a microparticle polynucleotide molecule. In some embodiments, the nucleic acid molecule is connected to the microparticle via the microparticle polynucleotide molecule (e.g., via hybridization between complementary nucleotide sequences on the nucleic acid molecule and the microparticle polynucleotide molecule). In some embodiments, the microparticle polynucleotide molecule comprises a primer sequence. In some embodiments, the microparticle polynucleotide molecule comprises a barcode sequence. In some embodiments, the microparticle polynucleotide molecule comprises a Unique Molecular Identifier (UMI) nucleotide sequence. In some embodiments, the microparticle polynucleotide molecule comprises an oligo-dT sequence. In some embodiments, the microparticle polynucleotide molecule comprises: a) a primer sequence, b) a barcode sequence, c) a Unique Molecular Identifier (UMI) nucleotide sequence, d) an oligo-dT sequence, and e) the nucleic acid sequence; wherein the nucleic acid comprises a polyA nucleotide sequence, and wherein the microparticle is connected to a)-e) in the following order: microparticle--a)--b)--c)--d)--e); and wherein the polyA sequence is hybridized with the oligo-dT sequence. In some embodiments, the microparticle polynucleotide molecule comprises: a) a primer sequence, b) a barcode sequence, c) a Unique Molecular Identifier (UMI) nucleotide sequence, d) an oligo-dT sequence, and e) the nucleic acid sequence; wherein the nucleic acid comprises a polyA nucleotide sequence, and wherein the microparticle is connected to a)-e) in the following order: microparticle--a)--c)--b)--d)--e); and wherein the polyA sequence is hybridized with the oligo-dT sequence.

Regulatory Element Identifier Barcode

In some embodiments, any of the nucleic acid molecules disclosed herein comprise a nucleic acid barcode sequence that serves to identify the specific regulatory element with which it is associated. As described herein, the present methods enable the screening of numerous (e.g., 10 to 10⁴) REs (e.g., in vivo or in vitro) in order to identify REs that provide selective expression of a transgene of interest in a specific type and/or population of cells (e.g., neurons, cardiomyocytes, etc.) or cellular subtypes (e.g., GABAergic subtypes, such as GABAergic neurons that express glutamic acid decarboxylase 2 (GAD2), GAD1, NKX2.1, DLX1, DLX5, SST, PV or VIP). The ability to identify a RE that provides selective expression in a given cell type is made possible by the assignment (or tagging, matching, pairing) of a specific barcode sequence to a specific candidate RE. When transgene expression is detected in a cell (e.g., by expression of a reporter gene, such as a gene encoding EGFP), the barcode sequence present in that cell makes it possible to determine which specific candidate RE was present in that cell to drive expression of the transgene (e.g., EGFP). In certain embodiments, the barcode sequence is unique to a specific regulatory element. Thus, for every candidate regulatory element tested in the present methods, a unique barcode sequence is paired to each candidate regulatory element, enabling identification of each candidate regulatory element. In some embodiments, the disclosure provides for methods of expressing any of the nucleic acids disclosed herein. In some embodiments, expression of the nucleic acid involves the step of transcribing a transgene of interest in the nucleic acid, wherein the transgene is operably linked to a candidate RE. Because in some embodiments the candidate RE in the nucleic acid is not transcribed along with the transgene, the barcode sequence is particularly useful because it preserves information identifying the specific candidate RE that facilitated transcription of the transgene of interest in the nucleic acid. In particular embodiments, the barcode sequence is in a DNA nucleic acid molecule. In some embodiments, the barcode sequence is in an RNA nucleic acid molecule that was transcribed from any of the DNA nucleic acid molecules disclosed herein.

The size of the barcode sequence can range from about 4 to about 100, about 4 to about 50, about 4 to about 20, or about 6 to about 20 or more nucleotides in length. In certain embodiments, the length of a barcode sequence is 6, 7, 8, 9, 10, 11, 12, 13, 14, 15, 16, 17, 18, 19, 20 nucleotides or longer. In certain embodiments, the length of a barcode sequence is at least 6, at least 7, at least 8, at least 9, at least 10, at least 11, at least 12, at least 13, at least 14, at least 15, at least 16, at least 17, at least 18, at least 19, at least 20 nucleotides in length. In some embodiments, the barcode sequence is contiguous, i.e., in a single stretch of adjacent nucleotides, or in some embodiments, the barcode sequence is separated into two or more separate subsequences that are separated by 1 or more nucleotides. In certain embodiments, separated barcode subsequences can be from about 4 to about 16 nucleotides in length. In some embodiments, the barcode subsequence is 4, 5, 6, 7, 8, 9, 10, 11, 12, 13, 14, 15, 16 nucleotides or longer. In certain embodiments, the barcode subsequence may be at least 4, 5, 6, 7, 8, 9, 10, 11, 12, 13, 14, 15, 16 nucleotides or longer. In certain embodiments, the barcode subsequence may be at most 4, 5, 6, 7, 8, 9, 10, 11, 12, 13, 14, 15, 16 nucleotides or shorter. In some embodiments, the barcode sequence comprises 2, 3, 4, 5, 6, 7, 8, 9, 10, 11, 12, 13, 14, 15, 16, 17, 18, 19, or 20 barcode subsequences, wherein the barcode subsequences are at least 2 to 10 nucleotides in length. In some embodiments, the barcode sequence comprises 2, 3, 4, 5, 6, 7, 8, 9, 10, 11, 12, 13, 14, 15, 16, 17, 18, 19, or 20 barcode subsequences, wherein the barcode subsequences are at least 4 to 20 nucleotides in length. In some embodiments, there is one or more nucleotides between two or more barcode subsequences. In some embodiments, there are 1 to 200, 1 to 150, 1 to 100, 1 to 90, 1 to 80, 1 to 70, 1 to 60, 1 to 50, 1 to 40, 1 to 30, 1 to 20, 1 to 10, 5 to 200, 5 to 150, 5 to 100, 5 to 90, 5 to 80, 5 to 70, 5 to 60, 5 to 50, 5 to 40, 5 to 30, 5 to 20, t to 10, 10 to 200, 10 to 150, 10 to 100, 10 to 90, 10 to 80, 10 to 70, 10 to 60, 10 to 50, 10 to 40, 10 to 30, 10 to 20, 20 to 200, 20 to 150, 20 to 100, 20 to 90, 20 to 80, 20 to 70, 20 to 60, 20 to 50, 20 to 40, 20 to 30, 30 to 200, 30 to 150, 30 to 100, 30 to 90, 30 to 80, 30 to 70, 30 to 60, 30 to 50, 30 to 40, 50 to 200, 50 to 150, 50 to 100, 50 to 90, 50 to 80, 50 to 70, 50 to 60, 75 to 200, 75 to 150, 75 to 100, 75 to 90, 75 to 80, 80 to 200, 80 to 150, 80 to 100, or 80 to 90 nucleotides between two or more barcode subsequences. In some embodiments, the barcode comprises two barcode subsequences, wherein each barcode subsequence is from 4 to 20 nucleotides in length, and wherein the barcode subsequences are separated by 1 to 200, 1 to 150, 1 to 100, 1 to 90, 1 to 80, 1 to 70, 1 to 60, 1 to 50, 1 to 40, 1 to 30, 1 to 20, 1 to 10, 5 to 200, 5 to 150, 5 to 100, 5 to 90, 5 to 80, 5 to 70, 5 to 60, 5 to 50, 5 to 40, 5 to 30, 5 to 20, t to 10, 10 to 200, 10 to 150, 10 to 100, 10 to 90, 10 to 80, 10 to 70, 10 to 60, 10 to 50, 10 to 40, 10 to 30, 10 to 20, 20 to 200, 20 to 150, 20 to 100, 20 to 90, 20 to 80, 20 to 70, 20 to 60, 20 to 50, 20 to 40, 20 to 30, 30 to 200, 30 to 150, 30 to 100, 30 to 90, 30 to 80, 30 to 70, 30 to 60, 30 to 50, 30 to 40, 50 to 200, 50 to 150, 50 to 100, 50 to 90, 50 to 80, 50 to 70, 50 to 60, 75 to 200, 75 to 150, 75 to 100, 75 to 90, 75 to 80, 80 to 200, 80 to 150, 80 to 100, or 80 to 90 nucleotides. In some embodiments, the barcode comprises three barcode subsequences, wherein each barcode subsequence is from 4 to 20 nucleotides in length, and wherein the barcode subsequences are separated by 1 to 200, 1 to 150, 1 to 100, 1 to 90, 1 to 80, 1 to 70, 1 to 60, 1 to 50, 1 to 40, 1 to 30, 1 to 20, 1 to 10, 5 to 200, 5 to 150, 5 to 100, 5 to 90, 5 to 80, 5 to 70, 5 to 60, 5 to 50, 5 to 40, 5 to 30, 5 to 20, t to 10, 10 to 200, 10 to 150, 10 to 100, 10 to 90, 10 to 80, 10 to 70, 10 to 60, 10 to 50, 10 to 40, 10 to 30, 10 to 20, 20 to 200, 20 to 150, 20 to 100, 20 to 90, 20 to 80, 20 to 70, 20 to 60, 20 to 50, 20 to 40, 20 to 30, 30 to 200, 30 to 150, 30 to 100, 30 to 90, 30 to 80, 30 to 70, 30 to 60, 30 to 50, 30 to 40, 50 to 200, 50 to 150, 50 to 100, 50 to 90, 50 to 80, 50 to 70, 50 to 60, 75 to 200, 75 to 150, 75 to 100, 75 to 90, 75 to 80, 80 to 200, 80 to 150, 80 to 100, or 80 to 90 nucleotides. In some embodiments, the barcode comprises four barcode subsequences, wherein each barcode subsequence is from 4 to 20 nucleotides in length, and wherein the barcode subsequences are separated by 1 to 200, 1 to 150, 1 to 100, 1 to 90, 1 to 80, 1 to 70, 1 to 60, 1 to 50, 1 to 40, 1 to 30, 1 to 20, 1 to 10, 5 to 200, 5 to 150, 5 to 100, 5 to 90, 5 to 80, 5 to 70, 5 to 60, 5 to 50, 5 to 40, 5 to 30, 5 to 20, t to 10, 10 to 200, 10 to 150, 10 to 100, 10 to 90, 10 to 80, 10 to 70, 10 to 60, 10 to 50, 10 to 40, 10 to 30, 10 to 20, 20 to 200, 20 to 150, 20 to 100, 20 to 90, 20 to 80, 20 to 70, 20 to 60, 20 to 50, 20 to 40, 20 to 30, 30 to 200, 30 to 150, 30 to 100, 30 to 90, 30 to 80, 30 to 70, 30 to 60, 30 to 50, 30 to 40, 50 to 200, 50 to 150, 50 to 100, 50 to 90, 50 to 80, 50 to 70, 50 to 60, 75 to 200, 75 to 150, 75 to 100, 75 to 90, 75 to 80, 80 to 200, 80 to 150, 80 to 100, or 80 to 90 nucleotides. In some embodiments, the barcode comprises five or more barcode subsequences, wherein each barcode subsequence is from 4 to 20 nucleotides in length, and wherein the barcode subsequences are separated by 1 to 200, 1 to 150, 1 to 100, 1 to 90, 1 to 80, 1 to 70, 1 to 60, 1 to 50, 1 to 40, 1 to 30, 1 to 20, 1 to 10, 5 to 200, 5 to 150, 5 to 100, 5 to 90, 5 to 80, 5 to 70, 5 to 60, 5 to 50, 5 to 40, 5 to 30, 5 to 20, t to 10, 10 to 200, 10 to 150, 10 to 100, 10 to 90, 10 to 80, 10 to 70, 10 to 60, 10 to 50, 10 to 40, 10 to 30, 10 to 20, 20 to 200, 20 to 150, 20 to 100, 20 to 90, 20 to 80, 20 to 70, 20 to 60, 20 to 50, 20 to 40, 20 to 30, 30 to 200, 30 to 150, 30 to 100, 30 to 90, 30 to 80, 30 to 70, 30 to 60, 30 to 50, 30 to 40, 50 to 200, 50 to 150, 50 to 100, 50 to 90, 50 to 80, 50 to 70, 50 to 60, 75 to 200, 75 to 150, 75 to 100, 75 to 90, 75 to 80, 80 to 200, 80 to 150, 80 to 100, or 80 to 90 nucleotides.

In some embodiments, one or more barcode sequences can be included in more than one region of the nucleic acid molecule. For example, one or more barcode sequences can be included in a coding region (e.g., sequence encoding the expressed transgene) or non-coding region (e.g., UTR and/or intronic sequence), or both. In some embodiments, neither the coding region nor the non-coding region of the transgene comprises the barcode sequence. In some embodiments, the barcode sequence is linked to the coding region or non-coding region of the transgene. In some embodiments, if more than one barcode sequence is included within the nucleic acid molecule, each barcode sequence can be identical (e.g., three copies of the same barcode sequence separated by at least 1 nucleotide), each can be different from each other (e.g., three different barcode sequences separated by at least 1 nucleotide), or some of the barcode sequences can be identical to and different from each other. Thus, any number of barcode sequences (identical, each different, or some identical/some different) can be included in any of the nucleic acid molecules disclosed herein. In certain embodiments, the nucleic acid molecule comprises at least 1, at least 2, at least 3, at least 4, at least 5, or at least 6 identical barcode sequences. In certain embodiments, the nucleic acid molecule comprises at least 1, at least 2, at least 3, at least 4, at least 5, or at least 6 different barcode sequences.

In some embodiments, a barcode sequence is specific to a specific candidate regulatory element. In some embodiments, a combination of barcode sequences is specific to a specific candidate regulatory element. In some embodiments, the placement of a barcode sequence in a nucleic acid molecule is specific to a specific candidate regulatory element. In some embodiments, a) a barcode sequence, b) a combination of barcode sequences, c) the placement of a barcode sequence in a nucleic acid molecule, or any combinations of a)-c) is specific to a specific candidate regulatory element.

In some embodiments, the coding region (e.g., the transgene) of any of the nucleic acid molecules comprises one or more barcode sequences. In some embodiments, the barcode in the coding region of the transgene comprises alternative codons. Alternative codons refer to synonymous codons in coding DNA. The genetic code is described as degenerate, or redundant, because a single amino acid may be coded for by more than one codon. For example, the codon TAT and codon TAC both encode the amino acid tyrosine. Thus, by way of example, a barcode placed in a coding region of a nucleotide sequence encoding EGFP can be designed to encode a region of EGFP using alternative codons (e.g., a change to the DNA sequence) while maintaining expression of the EGFP wildtype protein sequence (i.e., the alternative codons within the barcode sequence present in the coding region of an EGFP-encoding nucleotide sequence does not alter the EGFP amino acid sequence encoded by that nucleotide sequence). In some embodiments, a non-coding region (e.g., the UTR and/or intronic region of the transgene) of any of the nucleic acid molecules disclosed herein comprises one or more barcode sequences. In some embodiments, a non-coding region and a coding region of any of the nucleic acid molecules disclosed herein each comprises one or more barcode sequences. In some embodiments, any of the nucleic acid molecules disclosed herein comprises at least one barcode sequence that is at least partially in a coding region of the nucleic acid molecule and at least partially in a non-coding region of the nucleic acid molecule.

Generally, one or more barcode sequences can be placed anywhere in the nucleic acid molecule. In some embodiments, any of the nucleic acid sequences disclosed herein comprises a polyA tail and at least one barcode sequence. In some embodiments, the barcode sequence is located within about 25, 30, 35, 50, 100, 150, 200, 250, 300, 350, 400, 450 or 500 bases from the start of the polyA tail in the nucleic acid. In some embodiments, the barcode is located within about 50 bases from the start of the polyA tail in the nucleic acid. In some embodiments, the nucleic acid comprises multiple barcodes, wherein each barcode is separated by 80 to 120 bp within a region spanning about 50 bases proximal to the polyA tail in the nucleic acid. In some embodiments, at least one barcode sequence is placed in each 80 to 120 bp span within a region spanning about 50 bases proximal to the polyA tail.

Transgenes

In some embodiments, any of the nucleic acid molecules provided herein that can be used according to the present methods comprises a transgene sequence operably linked to a candidate regulatory element for use in the multiplex methods. In some embodiments, the transgenes of the present compositions and methods serve as reporters for detecting expression, if any, driven by the candidate regulatory element. In some embodiments, the candidate RE is located upstream of the transgene. In some embodiments, the candidate RE is located within a non-coding region of the transgene.

In some embodiments, the transgene is derived from a wildtype reference gene sequence (e.g., a gene sequence encoding an EGFP protein). In some embodiments, the transgene is at least 60%, 65%, 70%, 75%, 80%, 85%, 90%, 91%, 92%, 93%, 94%, 95%, 96%, 97%, 98%, 99% or 100% identical to a wildtype gene sequence. In some embodiments, the transgene does not comprise any mutations as compared to a wildtype reference nucleotide sequence. In some embodiments, the transgene is linked to any one or more of the barcode sequences disclosed here (i.e., the barcode sequence is not in the coding or non-coding region of the transgene). Any transgene of interest can be designed and used in the present methods. As described and exemplified herein, transgenes can be designed to include readily detectable and/or identifiable properties, characteristics or moieties. In some embodiments, the transgene comprises a modified nucleotide sequence (e.g., alternative codons) as compared to a reference nucleotide sequence. In some embodiments, the transgene can be designed to have certain beneficial properties, e.g., the expressed transgene specifically localizes to a particular compartment of a cell and/or the expressed transgene facilitates isolation and/or purification of the transgene protein, cell or cell component (e.g., nucleus). Various methods of protein design incorporating functional domains and/or tags known in the art can be used to generate a transgene useful in a specific context for the present methods. In some embodiments, the transgene is a DNA nucleic acid molecule. In some embodiments, the transgene is an RNA nucleic acid molecule that has been transcribed from any of the DNA nucleic acid molecules described herein.

In some embodiments, the transgene comprises a sequence encoding a reporter gene. Various reporter genes known in the art can be used to generate a transgene for the present methods. Reporter genes include any gene or nucleotide sequence that facilitates detection of the transgene expression, if any. A reporter gene can optionally allow for the localization of the expressed product, e.g., in a specific region or organelle of a cell and/or in a specific cell, tissue, organ or any part of a multicellular organism. Such reporter genes can also be designed such that they encode a fusion protein comprising a reporter polypeptide (e.g., a GFP protein) and one or more domains conferring a functional benefit, e.g., cell isolation, cell identification, or reporter localization to a region of a cell (e.g., via a nuclear binding domain). In some embodiments, any of the reporter genes disclosed herein encode one or more fluorescent protein such as green fluorescent protein (GFP), enhanced green fluorescent protein (EGFP), a yellow fluorescent protein (YFP), such as mBanana, a red fluorescent protein (RFP), such as mCherry, DsRed, dTomato, tdTomato, mHoneydew, mStrawberry, TagRFP, far-red fluorescent pamidronate (FRFP), such as mGrape1 or mGrape2, cyan fluorescent protein (CFP), blue fluorescent protein (BFP), enhanced cyan fluorescent protein (ECFP), ultramarine fluorescent protein (UMFP), orange fluorescent protein (OFP), such as mOrange or mTangerine, red (orange) fluorescent protein (mROFP), TagCFP, or a tetracystein fluorescent motif. In certain embodiments, the fluorescent protein is GFP or EGFP. In some embodiments, the transgene encodes a detectably labeled protein, such as a detectably labeled antibody or antigen-binding fragment thereof. In some embodiments, the transgene encodes a protein that may be detected using one or more agents that bind to the protein. For example, in some embodiments, the transgene encodes a protein that may be detected with one or more detectably labeled antibodies (e.g., a fluorescently labeled antibody).

As exemplified herein, the transgene can comprise a reporter gene sequence (e.g., a sequence encoding EGFP) operably linked to a sequence encoding a nuclear binding domain (e.g., a KASH domain or SUN domain protein, or biologically active fragment thereof), which targets the expressed reporter gene protein (EGFP) to the outer nuclear membrane. While the EGFP enables easy identification and sorting of cells expressing the transgene, the nuclear binding domain facilitates nuclei isolation from cells, which is beneficial for certain cells (e.g., neurons or adipocytes) that are prone to cell membrane disruption during dissociation from intact tissue. As those of skill in the art will recognize, a polypeptide encoded by a reporter gene sequence need not be linked to a nuclear binding domain sequence. In some embodiments, the polypeptide encoded by the reporter gene (e.g., EGFP) can be used alone to label the cytosol of the cell expressing the reporter gene, allowing for the identification of cells expressing the transgene. This labelling can be used to isolate whole cells from tissues which are not as prone to disruption of the cell membrane during dissociation from intact tissues (e.g., epithelial cells and fibroblasts). Such cells can be separated from their source (e.g., tissue), sorted based on reporter gene expression, and their transcriptome sequenced for analysis as detailed herein.

In some embodiments, the transgene comprises a sequence encoding a cell localization domain. Various cell localization domains are known in the art, and include, e.g., a KASH domain, SUN domain. The skilled worker is aware of other cell localization domains, such as those stored in the LOCATE subcellular localization database (http://locate.imb.uq.edu.au).

Regulatory Elements

In some embodiments, any of the nucleic acid molecules of the present disclosure include, e.g., one or more barcode sequences, and one or more candidate regulatory element operably linked to a transgene. As described herein, the present disclosure relates, in part, to a method of screening numerous (e.g., 10 to 10⁴) candidate REs (e.g., in vivo or in vitro) in order to identify REs that provide selective expression of a transgene of interest in a specific population of cells. Candidate REs can be tested using the methods described herein in order to identify REs which provide selective expression of a transgene in a given cell type (a cell type of interest or target cell). Generally, any known, natural, and/or synthetic candidate REs can be screened, isolated, and identified using the methods described herein. Known and/or naturally-occurring REs can be readily obtained for use as candidate REs in the present methods. Synthetic candidate REs useful for the present disclosure can be designed and generated using various methods known in the art. In some embodiments, candidate REs that can be used in the present methods can be REs with known activity in one or more cell types, but unknown in other cell types. In some embodiments, candidate REs that can be used in the present methods can be REs with unknown activity. Various known or novel (e.g., synthetic) REs can be screened according to the present methods to identify cell types in which the RE provides selective expression, as described herein. In some embodiments, a candidate RE that can be used in the present methods include known REs that can be used as negative or positive control REs against which candidate REs can be compared (e.g., pan-cellular REs).

In particular embodiments, the candidate RE is part of a DNA nucleic acid molecule. In some embodiments, the DNA nucleic acid molecule comprises any of the transgenes disclosed herein, one or more candidate REs, and one or more barcode sequences, wherein the barcode sequence correlates with the candidate RE in the nucleic acid (e.g., the barcode can be used to identify the RE contained in the nucleic acid molecule). In some embodiments, the disclosure provides for an RNA nucleic acid molecule transcribed from any of the DNA nucleic acid molecules disclosed herein (e.g., a DNA nucleic acid molecule comprising a barcode sequence(s), candidate RE(s) and transgenes as disclosed herein), wherein the RNA nucleic acid molecule comprises a transgene and a barcode sequence, and wherein the barcode sequence in the RNA molecule correlates with the candidate RE in the DNA molecule.

REs can function at the DNA and/or the RNA level. REs can function to modulate or control cell-selective (cell-specific) gene expression. REs can function to modulate gene expression at the transcriptional phase, post-transcriptional phase, or translational phase of gene expression. REs include, but are not limited to, promoter, enhancer, intronic, or other non-coding sequences. At the RNA level, regulation can occur at the level of translation (e.g., stability elements that stabilize mRNA for translation), RNA cleavage, RNA splicing, and/or transcriptional termination. In some embodiments, REs can recruit transcriptional factors that increase gene expression selectively in a cell type of interest. In some embodiments, REs can increase the rate at which RNA transcripts are produced, increase the stability of RNA produced, and/or increase the rate of protein synthesis from RNA transcripts.

REs are nucleic acid sequences or genetic elements which are capable of influencing (e.g., increasing or decreasing) expression of a gene or transgene (e.g., a reporter gene encoding a protein such as EGFP or luciferase; a transgene encoding a localization domain such as a KASH domain; and/or a therapeutic gene) in one or more cell types or tissues. In some embodiments, a RE can be an intron, a promoter, an enhancer, UTR, an inverted terminal repeat (ITR) sequence, a long terminal repeat sequence (LTR), stability element, posttranslational response element, micro RNA binding site, or a polyA sequence, or a combination thereof. In some embodiments, the RE is a promoter or an enhancer, or a combination thereof. In some embodiments, the RE is derived from a human sequence.

In some embodiments, two or more REs (known, natural and/or synthetic REs) can be combined to form a larger RE, which can be used as a candidate RE in the methods described herein. In some embodiments, it may be desirable to generate smaller candidate REs. Smaller REs that retain transgene expression activity are advantageous in gene therapy methods using large transgene, and/or where the cloning capacity of a vector or a plasmid is limited in view of the size of a transgene to be delivered using gene therapy. Thus, in some embodiments, candidate REs can be derived from REs with known activity by, e.g., truncating one or more bases at a time, and testing each resulting candidate RE for its ability to drive expression according to the present methods.

In some embodiments, two or more relatively short REs can be combined to form a larger RE and used as a candidate RE in the present methods. Such combinations have been previously shown to yield high transgene expression activity and/or size normalized gene expression. As such, this candidate RE can be screened to identify, e.g., in which cell type it can provide selective expression.

In some embodiments, a candidate RE disclosed herein comprises no more than 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 or 5000 bp.

In some embodiments, a candidate RE disclosed herein comprises no more than 40 bp, 45 bp, 49 bp, 50 bp, 56 bp, 60 bp, 70 bp, 80 bp, 90 bp, 100 bp, 110 bp, 117 bp, 120 bp, 130 bp, 140 bp, 150 bp, 160 bp, 170 bp, 180 bp, 190 bp, 200 bp, 210 bp, 220 bp, 230 bp, 240 bp, 250 bp, 259 bp, 260 bp, 265 bp, 270 bp, 280 bp, 290 bp, 300 bp, 310 bp, 320 bp, 330 bp, 340 bp, 350 bp, 360 bp, 370 bp, 380 bp, 390 bp, or 400 bp.

In some embodiments, a candidate RE that can be screened in the present methods is no more than 49 bp, 50 bp, 56 bp, 60 bp, 70 bp, 80 bp, 90 bp, 100 bp, 110 bp, 117 bp, 120 bp, 130 bp, 140 bp, 150 bp, 160 bp, 170 bp, 180 bp, 190 bp, 200 bp, 210 bp, 220 bp, 230 bp, 240 bp, 250 bp, 259 bp, 260 bp, 265 bp, 270 bp, 280 bp, 290 bp, 300 bp, 310 bp, 320 bp, 330 bp, 340 bp, 350 bp, 360 bp, 370 bp, 380 bp, 390 bp, or 400 bp. Such candidate REs may be useful for driving expression of a large transgene (e.g., in a gene therapy or an expression cassette) because the REs enhance transgene expression without taking up significant space within an AAV vector or expression cassette thus allowing greater capacity for a large transgene.

In some embodiments, the candidate RE described herein is 40-50 bp, 45-55 bp, 50-60 bp, or 55-65 bp. In some embodiments, the candidate RE is 45-60 bp. In some embodiments, the candidate RE described herein is 49 bp or 56 bp. In some embodiments, the candidate RE may be between 100 bp and 150 bp, between 110 bp and 140 bp, between 110 bp and 130 bp, or between 115 bp and 125 bp. In some embodiments, candidate REs are or are about 100 bp.

In some embodiments, candidate regulatory elements for use in the methods described herein can be selected using any method which allows for the identification of a candidate regulatory element (e.g., DNAase hypersensitivity, ATAC-Seq, and ChIP-Seq). See, e.g., WO 2018187363, which is incorporated herein by reference in its entirety. In some embodiments, regulatory elements may be identified using assay-based experiments (e.g., reporter gene assay), high-throughput experiments (e.g., a chromatin immunoprecipitation experiment), or computational approaches (e.g., ChIP-seq). See, e.g., Narlikar, et al., 2009, Briefings in Functional Genomics and Proteomics, 8(4): 215-230. In some embodiments, computational methodologies may be used to identify regulatory elements in a particular genome of interest (e.g., hg19). In some embodiments, putative insulator regions, which block the interaction between enhancers and promoters, may be identified and used to estimate the likely range of influence of genes and enhancers within a genomic region. See, e.g., Khan, et al., 2013, Genesis, 51:311-324. In some embodiments, phylogenetic footprinting can be used for computation prediction of cis-regulatory elements. In particular, phylogenetic footprinting can be used to identify conserved segments of DNA which may contain transcription factor finding sites which are retained throughout evolution. Id. In some embodiments, phylogenetic footprinting will be used only in regions defined by putative insulator regions, effectively allowing for the selection of candidate regulatory elements. Id.

In some embodiments, a candidate RE is derived from a known, control RE such as a known promoter. Examples of known, control promoters that can be used include, but are not limited to, a CMV promoter, a super core promoter, a TTR promoter, a Proto 1 promoter, a UCL-HLP promoter, an AAT promoter, a KAR promoter, a EF1a promoter, EFS promoter, or CMVe enhancer/CMV promoter combination, chicken β-actin promoter (CBA), CMV early enhancer/CBA promoter (CAG), elongation factor-1α promoter (EF1α), simian virus 40 promoter (SV40), phosphoglycerate kinase promoter (PGK), and the polyubiquitin C gene promoter (UBC). The level of expression from a transgene operably linked to such known, control REs can be analyzed against the level of expression from a transgene (the same transgene) operably linked to a candidate RE.

In some embodiments, a candidate RE can be a promoter that, when included in a nucleic acid molecule of the present disclosure, can drive transcription of a downstream sequence, which may be closely associated or in direct contact with the downstream sequence (e.g., a transgene). A promoter may drive high, medium, or low expression of a linked transgene.

In some embodiments, a candidate RE disclosed herein comprises a human-derived sequence. In some embodiments, a candidate RE of this disclosure is non-naturally occurring. In some embodiments, the candidate RE comprises a nucleotide sequence that has at least 80%, 90%, 95% or 99% sequence identity to a sequence in a human reference genome (or a human genome build). A homologous sequence may be a sequence which has a region with at least 80% sequence identity (e.g., as measured by BLAST) as compared to a region of the human genome. For example, a sequence that is at least 80%, at least 85%, at least 90%, at least 91%, at least 92%, at least 93%, at least 94%, at least 95%, at least 96%, at least 97%, at least 98%, or at least 99% homologous to a human sequence is deemed a human derived sequence.

In some embodiments, a human-derived candidate RE is a sequence that is 100% identical to a human sequence. In some embodiments, the sequence of a candidate RE is human derived, wherein the candidate RE differs from the corresponding human sequence by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, or 95 nucleotides or base pairs.

In some embodiments, at least 5%, 10%, 20%, 30%, 40%, 50%, 60%, 70%, 80%, 90%, 95%, 98%, or 99% of the candidate RE sequence is human derived. For example, a candidate RE can have 50% of its sequence be human derived, and the remaining 50% be non-human derived (e.g., mouse derived or fully synthetic). For further example, a candidate RE that is regarded as 50% human derived and comprises 300 bp may have an overall 45% sequence identity to a sequence in the human genome, while base pairs 1-150 of the candidate RE may have 90% identity (e.g., local sequence identity) to a similarly sized region of the human genome.

In some embodiments, a candidate RE contains a human-derived sequence and a non-human-derived sequence such that overall the RE has low sequence identity to the human genome. However, a part of the candidate RE has 100% sequence identity to the human genome. In other instances, at least 50%, 60%, 70%, 80%, 90%, 95%, 98% or 99% of the candidate RE sequence is human-derived or at least 10, 20, 30, 40, or 50 contiguous nucleotides are human-derived. For example, a candidate RE can have 50% of its sequence be human-derived, and the remaining 50% be non-human-derived (e.g., mouse derived, virus derived or fully synthetic).

The candidate RE can be derived from different species. In some embodiments, at least one part of a candidate RE is human-derived. Non-human-derived REs can be derived from mammalian, viral, or synthetic sequences.

As described herein, the present disclosure contemplates a method of identifying REs, wherein the RE can be operably linked to one or more functional sequences, including, e.g., transgenes described herein. Methods of effecting this operative linking, either before or after the DNA molecule is inserted into a vector, are well known.

In some embodiments, a candidate RE disclosed herein may be derived from a genomic promoter sequence. In some embodiments, a candidate RE disclosed herein may be derived from both a genomic promoter sequence and a 3′ untranslated region (3′ UTR). In some embodiments, a candidate RE disclosed herein may be derived from an intergenic sequence. In some embodiments, a candidate RE disclosed herein may be derived from a genomic sequence downstream of a gene, or from a 5′ UTR sequence, or a mixture of a 5′ UTR and downstream sequence.

In some embodiments, a candidate RE can be an enhancer, and its activity in an expression vector along with a promoter can be assessed for whether it provides selective expression (e.g., an increase or decrease of expression) of a transgene (e.g., EGFP) in a specific type of cell or specific population of cells as compared to expression of the same transgene by the promoter without the enhancer.

In some embodiments, a candidate RE herein is an intronic sequence, or comprises an intron, and its activity in an expression vector along with a promoter can be assessed for whether it provides selective expression of a transgene (e.g., a transgene encoding EGFP) in a specific population of cells as compared to expression of the same transgene by the promoter without the intronic sequence.

In some embodiments, a candidate RE herein is a promoter sequence, or comprises a promoter sequence, and it can be operably linked to a transgene of interest in a nucleic acid molecule of the present disclosure without any other promoter sequences and/or enhancer sequences to express the transgene.

In some embodiments, the candidate REs comprise part or all of a 5′ untranslated region (5′ UTR). 5′ UTR candidate REs can influence expression of a gene in several different ways. 5′ UTR candidate REs can contain binding sites for RNA binding proteins. Further, secondary structures formed by REs in the 5′ UTR can affect the binding of RNA binding proteins required for translation. In some examples, the candidate RE can have a high degree of secondary structure. In some embodiments, the candidate RE can have little or no secondary structure. The candidate RE can also contain an internal ribosome entry site (IRES), allowing for 5′ cap independent translation. The candidate RE can contain an upstream translation initiation codon (uAUG). In some embodiments, the candidate RE does not contain an upstream translation initiation codon. In some embodiments, the candidate RE does not contain any codon within one base of an AUG codon, or contains fewer codons similar to an AUG codon than expected by chance. In some embodiments, the candidate RE can contain an upstream open reading frame, which occurs when an upstream AUG (or sufficiently similar sequence) is present, followed by an in frame stop codon. In some examples, the candidate RE does not comprise an uORF. In some embodiments, the candidate REs contain microRNA binding sites, or binding sites for RNA binding proteins.

In some embodiments, a candidate RE of the disclosure can also be a functional fragment of any of the above. When the functional fragment is an enhancer, intronic sequence, a promoter sequence, or a combination thereof, higher, lower or more selective expression is observed when the fragment is operably linked to a transgene, as compared to a similar vector or cassette without the functional fragment. In some embodiments, a fragment is less than or equal to 25 bp, 30 bp, 40 bp, 50 bp, 60 bp, 70 bp, 80 bp, 90 bp, 100 bp, or 110 bp in length. In some embodiments, a candidate RE of the present disclosure derived from a human promoter sequence can be used without a second promoter in vector.

In some embodiments, a candidate RE that is an intronic sequence can be coupled or operably linked to any promoter. In some embodiments, a candidate RE that is a promoter sequence can be coupled or operably linked to a transgene without any other promoter sequences. In some embodiments, a candidate RE comprising a promoter sequence and an intronic sequence can be coupled or operably linked to a transgene without any other promoter sequences. In some embodiments, a candidate RE comprising a promoter sequence and an enhancer sequence can be coupled or operably linked to a transgene without any other promoter sequences.

Microparticles

In some embodiments, the disclosure provides for a microparticle connected to any of the nucleic acid molecules disclosed herein. In particular embodiments, the nucleic acid molecule that is connected to the microparticle is an RNA molecule transcribed from any of the DNA nucleic acid molecules disclosed herein. In some embodiments, the RNA molecule comprises a transgene and a barcode sequence. In some embodiments, the DNA molecule comprises a regulatory element, wherein the barcode sequence in the RNA molecule correlates with the regulatory element in the DNA molecule. In some embodiments, the microparticle is a bead. In some embodiments, the microparticle is connected to a microparticle polynucleotide molecule. In some embodiments, the microparticle polynucleotide sequence comprises a primer sequence. In particular embodiments, the primer sequence facilitates amplification and/or expression of at least a portion of the microparticle polynucleotide sequence. In some embodiments, the primer sequence facilitates amplification and/or expression of at least a portion of the microparticle polynucleotide sequence and at least a portion of any of the nucleic acid molecules disclosed herein that are connected/hybridized to the microparticle polynucleotide sequence. In some embodiments, the microparticle polynucleotide comprises a barcode nucleotide sequence unique to the microparticle (e.g., bead). In some embodiments, each microparticle comprises two or more microparticle polynucleotides. In some embodiments, each of the two or more microparticle polynucleotides comprises a different Unique Molecular Identifier (UMI) nucleotide sequence. In some embodiments, the microparticle polynucleotide comprises an oligo-dT nucleotide sequence. In some embodiments, the oligo-dT sequence is capable of hybridizing to a polyA portion of any of the nucleic acid molecules disclosed herein. In some embodiments, the microparticle polynucleotide molecule comprises: a) a primer sequence, b) a barcode sequence, c) a Unique Molecular Identifier (UMI) sequence, d) an oligo-dT sequence, and e) any of the nucleic acid molecules disclosed herein. In some embodiments, the microparticle polynucleotide molecule comprises: a) a primer sequence, b) a barcode sequence, c) a Unique Molecular Identifier (UMI) sequence, d) an oligo-dT sequence, and e) any of the nucleic acid molecules disclosed herein; wherein the nucleic acid comprises a polyA nucleotide sequence, wherein the microparticle is connected to a)-e) in the following order: microparticle--a)--b)--c)--d)--e); and wherein the polyA sequence is hybridized with the oligo-dT sequence. In some embodiments, the microparticle polynucleotide molecule comprises: a) a primer sequence, b) a barcode sequence, c) a Unique Molecular Identifier (UMI) sequence, d) an oligo-dT sequence, and e) any of the nucleic acid molecules disclosed herein; wherein the nucleic acid comprises a polyA nucleotide sequence, wherein the microparticle is connected to a)-e) in the following order: microparticle--a)--c)--b)--d)--e); and wherein the polyA sequence is hybridized with the oligo-dT sequence.

Delivery Methods and Compositions

In some embodiments, the disclosure provides for a vector (e.g., any of the vectors disclosed herein) comprising any of the nucleic acid molecules disclosed herein. In some embodiments, the vector is a viral vector (e.g., an adeno-associated viral vector). In some embodiments, the vector is a viral particle. In some embodiments, the vector is a non-viral vector.

In some embodiments, the nucleic acid molecules described herein are provided (or delivered) to cells or tissue, in vitro or in vivo, using various known and suitable methods available in the art. Conventional viral and non-viral based gene delivery methods can be used to introduce the nucleic acid molecules disclosed herein into cells (e.g., mammalian cells) and target tissues. Non-viral expression vector systems include nucleic acid vectors such as, e.g., linear oligonucleotides and circular plasmids; artificial chromosomes such as human artificial chromosomes (HACs), yeast artificial chromosomes (YACs), and bacterial artificial chromosomes (BACs or PACs)); episomal vectors; transposons (e.g., PiggyBac); and cosmids. Viral vector delivery systems include DNA and RNA viruses, such as, e.g., retroviral vectors, lentiviral vectors, adenoviral vectors, and adeno-associated viral vectors. Methods of incorporating the nucleic acid molecules described herein into any of the non-viral and viral expression systems are known to those of skill in the art.

Methods and compositions for non-viral delivery of nucleic acids are known in the art, including physical and chemical methods. Physical methods generally refer to methods of delivery employing a physical force to counteract the cell membrane barrier in facilitating intracellular delivery of genetic material. Examples of physical methods include the use of a needle, ballistic DNA, electroporation, sonoporation, photoporation, magnetofection, and hydroporation. Chemical methods generally refer to methods in which chemical carriers deliver a nucleic acid molecule to a cell and may include inorganic particles, lipid-based carriers, polymer-based carriers and peptide-based carriers.

In some embodiments, a non-viral expression vector is administered to a target cell using an inorganic particle. Inorganic particles may refer to nanoparticles, such as nanoparticles that are engineered for various sizes, shapes, and/or porosity to escape from the reticuloendothelial system or to protect an entrapped molecule from degradation. Inorganic nanoparticles can be prepared from metals (e.g., iron, gold, and silver), inorganic salts, or ceramics (e.g., phosphate or carbonate salts of calcium, magnesium, or silicon). The surface of these nanoparticles can be coated to facilitate DNA binding or targeted gene delivery. Magnetic nanoparticles (e.g., supermagnetic iron oxide), fullerenes (e.g., soluble carbon molecules), carbon nanotubes (e.g., cylindrical fullerenes), quantum dots and supramolecular systems may also be used.

In some embodiments, a non-viral expression vector is administered to a target cell using a cationic lipid (e.g., cationic liposome). Various types of lipids have been investigated for gene delivery, such as, for example, a lipid nano emulsion (e.g., which is a dispersion of one immiscible liquid in another stabilized by emulsifying agent) or a solid lipid nanoparticle. In some embodiments, a non-viral expression vector can be delivered using lipid nanoparticles (LNPs). In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2017/173054, WO2015/095340, and WO2014/136086, as well as references provided therein.

In some embodiments, a non-viral expression vector is administered to a target cell using a peptide based delivery vehicle. Peptide based delivery vehicles can have advantages of protecting the genetic material to be delivered, targeting specific cell receptors, disrupting endosomal membranes and delivering genetic material into a nucleus. In some embodiments, a non-viral expression vector is administered to a target cell using a polymer based delivery vehicle. Polymer based delivery vehicles may comprise natural proteins, peptides and/or polysaccharides or synthetic polymers. In one embodiment, a polymer based delivery vehicle comprises polyethylenimine (PEI). PEI can condense DNA into positively charged particles which bind to anionic cell surface residues and are brought into the cell via endocytosis. In other embodiments, a polymer based delivery vehicle may comprise poly-L-lysine (PLL), poly (DL-lactic acid) (PLA), poly (DL-lactide-co-glycoside) (PLGA), polyornithine, polyarginine, histones, protamines, dendrimers, chitosans, synthetic amino derivatives of dextran, and/or cationic acrylic polymers. In certain embodiments, polymer based delivery vehicles may comprise a mixture of polymers, such as, for example PEG and PLL.

In some embodiments, any of the nucleic acid molecules disclosed herein comprise a candidate regulatory element operably linked to a transgene and barcode sequence and can be delivered using any known suitable viral vector including, e.g., retroviruses (e.g., A-type, B-type, C-type, and D-type viruses), adenovirus, parvovirus (e.g. adeno-associated viruses or AAV), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Examples of retroviruses include avian leukosis-sarcoma virus, human T-lymphotrophic virus type 1 (HTLV-1), bovine leukemia virus (BLV), lentivirus, and spumavirus. Other viruses include Norwalk virus, togavirus, flavivirus, reoviruses, papovavirus, hepadnavirus, and hepatitis virus, for example. Viral vectors may be classified into two groups according to their ability to integrate into the host genome—integrating and non-integrating. Oncoretroviruses and lentiviruses can integrate into host cellular chromatin while adenoviruses, adeno-associated viruses, and herpes viruses predominantly persist in the cell nucleus as extrachromosomal episomes.

In some embodiments, a suitable viral vector is a retroviral vector. Retroviruses refer to viruses of the family Retroviridae. Examples of retroviruses include oncoretroviruses, such as murine leukemia virus (MLV), and lentiviruses, such as human immunodeficiency virus 1 (HIV-1). Retroviral genomes are single-stranded (ss) RNAs and comprise various genes that may be provided in cis or trans. For example, a retroviral genome may contain cis-acting sequences such as two long terminal repeats (LTR), with elements for gene expression, reverse transcription and integration into the host chromosomes. Other components include the packaging signal (psi or ψ), for the specific RNA packaging into newly formed virions and the polypurine tract (PPT), the site of the initiation of the positive strand DNA synthesis during reverse transcription. In addition, in some embodiments, the retroviral genome may comprise gag, pol and env genes. The gag gene encodes the structural proteins, the pol gene encodes the enzymes that accompany the ssRNA and carry out reverse transcription of the viral RNA to DNA, and the env gene encodes the viral envelope. Generally, the gag, pol and env are provided in trans for viral replication and packaging.

In some embodiments, a retroviral vector provided herein may be a lentiviral vector. At least five serogroups or serotypes of lentiviruses are recognized. Viruses of the different serotypes may differentially infect certain cell types and/or hosts. Lentiviruses, for example, include primate retroviruses and non-primate retroviruses. Primate retroviruses include HIV and simian immunodeficiency virus (SIV). Non-primate retroviruses include feline immunodeficiency virus (FIV), bovine immunodeficiency virus (BIV), caprine arthritis-encephalitis virus (CAEV), equine infectious anemia virus (EIAV) and visnavirus. Lentiviruses or lentivectors may be capable of transducing quiescent cells. As with oncoretrovirus vectors, the design of lentivectors may be based on the separation of cis- and trans-acting sequences.

In some embodiments, the present disclosure provides expression vectors that have been designed for delivery by an optimized therapeutic retroviral vector. The retroviral vector can be a lentivirus comprising any one or more of: a left (5′) LTR; sequences which aid packaging and/or nuclear import of the virus; a promoter; optionally one or more additional regulatory elements (such as, for example, an enhancer or polyA sequence); optionally a lentiviral reverse response element (RRE); a construct comprising a candidate regulatory element operably linked to a transgene (e.g. EGFP-KASH); optionally an insulator; and a right (3′) retroviral LTR.

In some embodiments, a viral vector provided herein is an adeno-associated virus (AAV). AAV is a small, replication-defective, non-enveloped animal virus that infects humans and some other primate species. AAV is not known to cause human disease and induces a mild immune response. AAV vectors can also infect both dividing and quiescent cells without integrating into the host cell genome.

The AAV genome naturally consists of a linear single stranded DNA which is ˜4.7 kb in length. The genome consists of two open reading frames (ORF) flanked by an inverted terminal repeat (ITR) sequence that is about 145 bp in length. The ITR consists of a nucleotide sequence at the 5′ end (5′ ITR) and a nucleotide sequence located at the 3′ end (3′ ITR) that contain palindromic sequences. The ITRs function in cis by folding over to form T-shaped hairpin structures by complementary base pairing that function as primers during initiation of DNA replication for second strand synthesis. The two open reading frames encode for rep and cap genes that are involved in replication and packaging of the virion. In some embodiments, an AAV vector provided herein does not contain the rep or cap genes. Such genes may be provided in trans for producing virions as described further below.

In some embodiments, an AAV vector may include a stuffer nucleic acid. In some embodiments, the stuffer nucleic acid may encode a green fluorescent protein or antibiotic resistance gene providing resistance to antibiotics such as kanamycin or ampicillin. In certain embodiments, the stuffer nucleic acid may be located outside of the ITR sequences (e.g., as compared to the transgene sequence and regulatory sequences, which are located between the 5′ and 3′ ITR sequences).

In some embodiments, the AAV vector is any one of AAV1, AAV2, AAV3, AAV3b, AAV4, AAV5, AAV6, AAV7, AAV8, AAV9, AAV10, AAV 11, AAV12, AAV13, AAV-DJ, AAV-DJ8, AAV-DJ9 or a chimeric, hybrid, or variant AAV. The AAV can also be a self-complementary AAV (scAAV). These serotypes differ in their tropism, or the types of cells they infect. In some embodiments, the AAV vector comprises the genome and capsids from multiple serotypes (e.g., pseudotypes). For example, an AAV may comprise the genome of serotype 2 (e.g., ITRs) packaged in the capsid from serotype 5 or serotype 9. Pseudotypes may improve transduction efficiency as well as alter tropism. In some embodiments, the AAV is an AAV9 serotype. In certain embodiments, an expression vector designed for delivery by an AAV comprises a 5′ ITR and a 3′ ITR.

In some embodiments, the ITRs of AAV serotype 6 or AAV serotype 9 can be used in any of the AAV vectors disclosed herein. However, ITRs from other suitable serotypes may be selected. In some embodiments, any of the nucleic acid molecules disclosed herein is packaged into a capsid protein and delivered to a selected host cell. AAV vectors of the present disclosure may be generated from a variety of adeno-associated viruses. The tropism of the vector may be altered by packaging the recombinant genome of one serotype into capsids derived from another AAV serotype. In some embodiments, the ITRs of the rAAV virus can be based on the ITRs of any one of AAV1-12 and may be combined with an AAV capsid selected from any one of AAV1-12, AAV-DJ, AAV-DJ8, AAV-DJ9 or other modified serotypes. In particular embodiments, the AAV ITRs and/or capsids are selected based on the cell or tissue to be targeted with the AAV vector.

In some embodiments, the disclosure provides for a vector comprising any of the nucleic acids disclosed herein, wherein the vector is an AAV vector or an AAV viral particle, or virion. In some embodiments, an AAV vector or an AAV viral particle, or virion, can be used to deliver any of the nucleic acid molecules disclosed herein comprising any of the candidate regulatory elements disclosed herein operably linked to any of the transgenes disclosed herein, either in vivo, ex vivo, or in vitro. In some embodiments, such an AAV vector is replication-deficient. In some embodiments, an AAV virus is engineered or genetically modified so that it can replicate and generate virions only in the presence of helper factors.

In some embodiments, one or more candidate regulatory elements operably linked to a transgene can be screened using methods described herein to determine if the candidate regulatory element provides selective (e.g., increased or decreased) expression of the transgene in a target cell, cell type, or tissue. In some embodiments, an expression vector designed for delivery by an AAV comprises a 5′ ITR, a promoter, a nucleic acid molecule comprising a candidate regulatory element operably linked to a transgene (e.g. a transgene encoding EGFP-KASH) and a barcode sequence, and a 3′ ITR. In some embodiments, an expression vector designed for delivery by an AAV comprises a 5′ ITR, an enhancer, a promoter, a nucleic acid molecule comprising a candidate regulatory element operably linked to a transgene (e.g. a transgene encoding EGFP-KASH) and a barcode sequence, a polyA sequence, and a 3′ ITR.

In some embodiments, the present disclosure provides for a viral vector comprising any of the nucleic acids disclosed herein. The terms “viral particle”, and “virion” are used herein interchangeably and relate to an infectious and typically replication-defective virus particle comprising the viral genome (e.g., the viral expression vector) packaged within a capsid and, as the case may be e.g., for retroviruses, a lipidic envelope surrounding the capsid. A “capsid” refers to the structure in which the viral genome is packaged. A capsid consists of several oligomeric structural subunits made of proteins. For example, AAV have an icosahedral capsid formed by the interaction of three capsid proteins: VP1, VP2 and VP3. In some embodiments, a virion provided herein is a recombinant AAV virion obtained by packaging an AAV vector that comprises a candidate regulatory element operably linked to a transgene and barcode sequence, as described herein, in a protein shell.

In some embodiments, a recombinant AAV virion provided herein may be prepared by encapsidating an AAV genome derived from a particular AAV serotype in a viral particle formed by natural Cap proteins corresponding to an AAV of the same particular serotype. In other embodiments, an AAV viral particle provided herein comprises a viral vector comprising ITR(s) of a given AAV serotype packaged into proteins from a different serotype. See e.g., Bunning H et al. J Gene Med 2008; 10: 717-733. For example, a viral vector having ITRs from a given AAV serotype may be packaged into: a) a viral particle constituted of capsid proteins derived from a same or different AAV serotype (e.g. AAV2 ITRs and AAV9 capsid proteins; AAV2 ITRs and AAV8 capsid proteins; etc.); b) a mosaic viral particle constituted of a mixture of capsid proteins from different AAV serotypes or mutants (e.g. AAV2 ITRs with AAV1 and AAV9 capsid proteins); c) a chimeric viral particle constituted of capsid proteins that have been truncated by domain swapping between different AAV serotypes or variants (e.g. AAV2 ITRs with AAV8 capsid proteins with AAV9 domains); or d) a targeted viral particle engineered to display selective binding domains, enabling stringent interaction with target cell specific receptors (e.g. AAV5 ITRs with AAV9 capsid proteins genetically truncated by insertion of a peptide ligand; or AAV9 capsid proteins non-genetically modified by coupling of a peptide ligand to the capsid surface).

The skilled person will appreciate that an AAV virion provided herein may comprise capsid proteins of any AAV serotype. In one embodiment, the viral particle comprises capsid proteins from an AAV serotype selected from the group consisting of an AAV1, an AAV2, an AAV5, an AAV6, an AAV8, and an AAV9.

Numerous methods are known in the art for production of rAAV virions, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids. In some embodiments, rAAV production cultures for the production of rAAV virus particles comprise; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a nucleic acid molecule comprising a candidate regulatory element operably linked to a transgene (e.g., a nucleotide sequence encoding a nuclear binding domain operably linked to a reporter gene sequence as described herein), flanked by AAV ITR sequences; wherein the nucleic acid molecule comprises one or more barcode sequences, and 5) suitable media and media components to support rAAV production.

In some embodiments, the producer cell line is an insect cell line (typically Sf9 cells) that is infected with baculovirus expression vectors that provide Rep and Cap proteins. This system does not require adenovirus helper genes (Ayuso E, et al., Curr. Gene Ther. 2010, 10:423-436).

The term “cap protein”, as used herein, refers to a polypeptide having at least one functional activity of a native AAV Cap protein (e.g. VP1, VP2, VP3). Examples of functional activities of cap proteins include the ability to induce formation of a capsid, facilitate accumulation of single-stranded DNA, facilitate AAV DNA packaging into capsids (i.e. encapsidation), bind to cellular receptors, and facilitate entry of the virion into host cells. In principle, any Cap protein can be used in the context of the present invention.

Cap proteins have been reported to have effects on host tropism, cell, tissue, or organ specificity, receptor usage, infection efficiency, and immunogenicity of AAV viruses. Accordingly, an AAV cap for use in an rAAV may be selected taking into consideration, for example, the subject's species (e.g. human or non-human), the subject's immunological state, the subject's suitability for long or short-term treatment, or a particular therapeutic application (e.g. treatment of a particular disease or disorder, or delivery to particular cells, tissues, or organs). In certain embodiments, the cap protein is derived from the AAV of the group consisting of AAV1, AAV2, AAV5, AAV6, AAV8, and AAV9 serotypes.

In some embodiments, an AAV Cap for use in the methods provided herein can be generated by mutagenesis (i.e., by insertions, deletions, or substitutions) of one of the aforementioned AAV caps or its encoding nucleic acid. In some embodiments, the AAV cap is at least 70%, 75%, 80%, 85%, 90%, 95%, 98%, or 99% or more similar to one or more of the aforementioned AAV caps.

In some embodiments, the AAV cap is chimeric, comprising domains from two, three, four, or more of the aforementioned AAV caps. In some embodiments, the AAV cap is a mosaic of VP1, VP2, and VP3 monomers originating from two or three different AAV or a recombinant AAV. In some embodiments, a rAAV composition comprises more than one of the aforementioned caps.

In some embodiments, an AAV cap for use in a rAAV virion is engineered to contain a heterologous sequence or other modification. For example, a peptide or protein sequence that confers selective targeting or immune evasion may be engineered into a cap protein. Alternatively or in addition, the cap may be chemically modified so that the surface of the rAAV is polyethylene glycolated (i.e., pegylated), which may facilitate immune evasion. The cap protein may also be mutagenized (e.g., to remove its natural receptor binding, or to mask an immunogenic epitope).

The term “rep protein”, as used herein, refers to a polypeptide having at least one functional activity of a native AAV rep protein (e.g., rep 40, 52, 68, 78). Examples of functional activities of a rep protein include any activity associated with the physiological function of the protein, including facilitating replication of DNA through recognition, binding and nicking of the AAV origin of DNA replication as well as DNA helicase activity. Additional functions include modulation of transcription from AAV (or other heterologous) promoters and site-specific integration of AAV DNA into a host chromosome. In some embodiments, AAV rep genes may be from the serotypes AAV1, AAV2, AAV4, AAV5, AAV6, AAV7, AAV8, AAV9, AAV10 or AAVrh10.

In some embodiments, an AAV rep protein for use in the method of the invention can be generated by mutagenesis (i.e. by insertions, deletions, or substitutions) of one of the aforementioned AAV reps or its encoding nucleic acid. In some embodiments, the AAV rep is at least 70%, 75%, 80%, 85%, 90%, 95%, 98%, or 99% or more similar to one or more of the aforementioned AAV reps.

The expressions “helper functions” or “helper genes”, as used herein, refer to viral proteins upon which AAV is dependent for replication. The helper functions include those proteins required for AAV replication including, without limitation, those proteins involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. Helper functions include, without limitation, adenovirus E1, E2a, VA, and E4 or herpesvirus UL5, ULB, UL52, and UL29, and herpesvirus polymerase. In a preferred embodiment, the proteins upon which AAV is dependent for replication are derived from adenovirus.

In some embodiments, a viral protein upon which AAV is dependent for replication for use in the method of the invention can be generated by mutagenesis (i.e. by insertions, deletions, or substitutions) of one of the aforementioned viral proteins or its encoding nucleic acid. In some embodiments, the viral protein is at least 70%, 75%, 80%, 85%, 90%, 95%, 98%, or 99% or more similar to one or more of the aforementioned viral proteins.

Methods for assaying the functions of cap proteins, rep proteins and viral proteins upon which AAV is dependent for replication are well known in the art.

In some embodiments, a viral expression vector can be associated with a lipid delivery vehicle (e.g., cationic liposome or LNPs as described here) for administering to a target cell.

The various delivery systems containing the nucleic acid molecules described herein can be administered to an organism for delivery to cells in vivo or administered to a cell or cell culture ex vivo. Administration is by any of the routes normally used for introducing a molecule into ultimate contact with blood, fluid, or cells including, but not limited to, injection, infusion, topical application and electroporation. Suitable methods of administering such nucleic acids are available and known to those of skill in the art.

The nucleic acid molecules can be delivered, in vitro, in vivo, or ex vivo to target various cells and/or tissues. In some embodiments, delivery can be targeted to various organs/tissues and corresponding cells, e.g., to the brain, heart, skeletal muscle, liver, kidney, spleen, or stomach. In some embodiments, the nucleic acid molecules are delivered to any one or more of neuronal cells, cardiomyocytes, skeletal muscle cells, smooth muscle cells, hepatocytes, podocytes, or epithelial cells. In some embodiments, delivery can be targeted to diseased cells, such as, e.g., tumor or cancer cells. In some embodiments, delivery can be targeted to stem cells, blood cells, or immune cells.

In some embodiments, the disclosure provides for a mixture of any of the vectors disclosed herein, or any of the nucleic acids disclosed herein. In some embodiments, the mixture comprises two or more nucleic acid molecules wherein each of the nucleic acid molecules comprises a different barcode nucleotide sequence. In some embodiments, the mixture comprises about 10¹ to about 10⁴ nucleic acid molecules, wherein each nucleic acid molecule comprises a different regulatory element. In some embodiments, the mixture comprises about 10¹ nucleic acid molecules, wherein each nucleic acid molecule comprises a different regulatory element. In some embodiments, the mixture comprises about 10² nucleic acid molecules, wherein each nucleic acid molecule comprises a different regulatory element. In some embodiments, the mixture comprises about 10³ nucleic acid molecules, wherein each nucleic acid molecule comprises a different regulatory element. In some embodiments, the mixture comprises about 10⁴ nucleic acid molecules, wherein each nucleic acid molecule comprises a different regulatory element. In some embodiments, the mixture or nucleic acid molecules comprises about 10, about 50, about 100, about 250, about 500, about 750, about 1000, about 1250, about 1500, about 1750, about 2000, about 2500, about 3000, about 3500, about 4000, about 4500, about 5000, about 5500, about 6000, about 6500, about 7000, about 7500, about 8000, about 8500, about 9000, about 9500, about 10000, or more different regulatory elements.

Methods of the Multiplex Assay

As described herein, the present disclosure relates, in part, to a high-throughput method of screening regulatory elements (e.g., in vivo or in vitro) in order to identify regulatory elements that provide selective expression of a transgene of interest in a specific population of cells.

In some embodiments, the methods include providing/treating two or more cells (e.g., a population of cells or tissue) with a mixture of vectors each comprising nucleic acid sequences comprising a candidate regulatory element operably linked to a sequence encoding a transgene (e.g., a transgene comprising a reporter gene and a barcode for regulatory element identification). In some embodiments, any of the methods disclosed herein may comprise the step of administering any of the nucleic acids or vectors disclosed herein to a population of cells. Administration is by any of the routes normally used for introducing a molecule into ultimate contact with a population of cells including, but not limited to, injection, infusion, topical application and electroporation. In some embodiments, the cells in the population of cells are mammalian cells. In some embodiments, the cells in the population of cells are human cells. In some embodiments, the population of cells is in vitro. In some embodiments, the population of cells is in vivo. In some embodiments, the population of cells is in a tissue or organ from an animal. In some embodiments, the population of cells is in an animal. In some embodiments, the animal is a mouse, rat, frog, dog, rabbit, guinea pig, or non-human primate. In some embodiments, the non-human primate is a cynomolgus monkey or a chimpanzee. In some embodiments, if the population of cells is in a tissue or organ in an animal, the tissue or organ (or a sample from the tissue or organ) is removed (e.g., surgically removed) from the animal to separate/isolate cells from the population of cells (as described in greater detail below). In some embodiments, the population of cells is in an animal, and the vector and/or nucleic acid is administered to the animal by any one or more of the following routes of administration: intravenous, subcutaneous, orally, intranasal, intramuscular, intraocular, direct injection into a tissue of interest, or intrathecal.

In some embodiments, in order to identify regulatory elements according to the present methods, individual cells of the treated cells or tissue are separated and isolated for further analysis to, e.g., assess transgene expression, determine the identity of each cell expressing the transgene, and/or correlate the expressed transgene with the originating regulatory element (e.g., using the barcode), as described below.

Single Cell RNA Isolation

In some embodiments, the disclosure provides for methods incorporating any method which allows for the isolation or separation of a single cell from a mixture of cells (e.g., cells from a tissue, organ, or body fluids (e.g., serum)). In some embodiments, each cell that expresses a transgene operably linked to a regulatory element is separated/isolated in order to sequence the transcriptome of each of the cells. Generally, various methods are known in the art for separating individual cells from a mixture of cells (e.g., cells from a tissue, organ, or body fluids (e.g., serum)). Such methods include, but are not limited to, separating cells based on buoyant density in a cell separation composition (U.S. Pat. No. 4,927,750), separating serological factors on density gradients using latex beads coated with antiserological factor (U.S. Pat. No. 3,862,303), separating cells through the use of a magnetic field (U.S. Pat. No. 4,777,145), and separating T and B cells on density gradients (U.S. Pat. No. 4,511,662). In some embodiments, the individual cells are separated based on fluorescent intensities emitted by a fluorescent marker within or bound to the cells, e.g., by using FACS sorting. Those skilled in the art can readily carry out a suitable process for a particular context or application. For example, cell membranes of certain cell types (e.g., neurons and adipocytes) are prone to disruption during dissociation from intact tissue. Thus, certain standard organ dissociation techniques (e.g., enzymatic and mechanical forces) are better suited for some cell types over others. In some cases, depending on the particular application, the cells are separated/isolated intact (e.g., without lysing). In some embodiments, the nuclei of the cells are separated/isolated intact (e.g., without lysing).

In some embodiments, individual cells can be isolated from a population of cells, such as from a tissue source. Examples of a tissue source that can be used in the present methods include connective tissue, muscular tissue, nervous tissue, and epithelial tissue. Examples of cells of connective tissue that can be separated/isolated and analyzed in the application of the present methods include, e.g., fibroblasts, adipocytes, macrophages, mast cells, plasma cells, etc. Examples of cells of muscular tissue that can be separated/isolated and analyzed in the application of the present methods include, e.g., cardiomyocytes, skeletal muscle cells, cardiac muscle cells, smooth muscle cells, etc. Examples of cells of nervous tissue that can be separated/isolated and analyzed in the application of the present methods include, e.g., neurons, glia, etc. Examples of cells of nervous tissue that can be separated/isolated and analyzed in the application of the present methods include subtypes of neuronal cells, such as GABAergic cells, including, e.g., GABAergic neurons that express glutamic acid decarboxylase 2 (GAD2), GAD1, NKX2.1, DLX1, DLX5, SST, PV or VIP. Examples of cells of epithelial tissue that can be separated/isolated and analyzed in the application of the present methods include, e.g., squamous epithelium, cuboidal epithelium, columnar epithelium, etc. In some embodiments, individual cells can be separated/isolated from blood cells. In some embodiments, individual cells can be separated/isolated from a population of stem cells, e.g., from bone marrow. In some embodiments, individual cells can be separated/isolated from a tumor. In some embodiments, individual cells can be separated/isolated from a cancer.

In some embodiments, the disclosure provides for methods incorporating any method which allows for the sorting of separated/isolated cells. In some embodiments, the separated/isolated cells (or nuclei) are sorted prior to undergoing single-cell RNA sequencing. In certain embodiments, cells are isolated and sorted based on, e.g., the expression of a transgene (e.g., a reporter gene encoding proteins such as EGFP or EGFP-KASH, as exemplified herein), presence of natural cell-specific markers, or presence of an added label. Various reporter genes, natural cell-specific markers, and labels for the purpose of cell sorting are known in the art, as described herein. As those of skill in the art would recognize, a reporter transgene or label can be designed to be expressed in any part of a cell (e.g., cell surface or surface of the nuclear envelope) as needed. For example, KASH proteins (Klarsicht, ANC-1, Syne homology) and SUN proteins (Sad1 and UNC-84), both of which are representative nuclear binding domain sequences, express and localize to the outer membrane of the nuclear envelope. As exemplified herein, expression of a transgene comprising a fluorescent marker and a nuclear binding domain sequence allows nuclei sorting based on the expression of the transgene. Various cell sorting methods such as fluorescence-activated cell sorting (FACS) and magnet-activated cell sorting (MACS) can be used in the practice of the present disclosure.

In some embodiments, the separated cells are not sorted prior to undergoing single-cell RNA sequencing.

In some embodiments, any labeling substances known to those skilled in the art can be utilized in combination with the cell sorting methods described above. In certain embodiments, cells can be isolated and sorted based on the expression of a reporter gene (e.g. expression of a fluorescent label such as EGFP). In some embodiments, the label is a fluorescent label. Examples of fluorescent labels include, but are not limited to, green fluorescent protein (GFP), enhanced green fluorescent protein (EGFP), a yellow fluorescent protein (YFP), such as mBanana, a red fluorescent protein (RFP), such as mCherry, DsRed, dTomato, tdTomato, mHoneydew, or mStrawberry, TagRFP, far-red fluorescent pamidronate (FRFP), such as mGrape1 or mGrape2, a cyan fluorescent protein (CFP), a blue fluorescent protein (BFP), enhanced cyan fluorescent protein (ECFP), ultramarine fluorescent protein (UMFP), orange fluorescent protein (OFP), such as mOrange or mTangerine, red (orange) fluorescent protein (mROFP), TagCFP, or a tetracystein fluorescent motif. In certain embodiments, the fluorescent label is GFP or EGFP. In some embodiments, the separated/isolated cell or nucleus is encapsulated in a droplet. In some embodiments, the droplet is an emulsion droplet. In some embodiments, the droplet is of a nanoliter-scale. In some embodiments, the droplet further comprises a microparticle. In some embodiments, the microparticle is a bead.

In some embodiments, the disclosure provides for methods incorporating any method of compartmentalizing cells or nuclei for further analysis of their mRNA transcripts. In some embodiments, the disclosure provides for a droplet (e.g., an emulsion droplet) comprising any of the nucleic acids disclosed herein. In some embodiments, the disclosure provides for an emulsion droplet comprising any of the cells disclosed herein. In some embodiments, the disclosure provides for a droplet (e.g., an emulsion droplet) comprising any of the microparticles disclosed herein. In some embodiments, the disclosure provides for a droplet (e.g., an emulsion droplet) comprising any of the microparticles disclosed herein and also any of the cells disclosed herein.

In some embodiments, once any of the cells or nuclei disclosed herein is encapsulated by any of the droplets disclosed herein, the cell or nucleus is lysed to release the contents of the cell or nucleus (e.g., the RNA contents) into the droplet. In particular embodiments, the cell or nucleus is lysed to release the contents of the cell or nucleus (e.g., the RNA contents) into the droplet, wherein the droplet further comprises any of the microparticles disclosed herein. In some embodiments, a plurality of RNA molecules is connected to a plurality of microparticles (e.g., beads), wherein each bead is uniquely barcoded. In some embodiments, the microparticle is connected to a microparticle polynucleotide, wherein the microparticle polynucleotide comprises an oligo-dT nucleotide sequence. In some embodiments, the oligo-dT nucleotide sequence is capable of hybridizing with the 3′ polyadenylated (poly(A)) tail of any of the mRNA molecules released from the lysed cell or nucleus. In some embodiments, RNA captured and isolated for analysis in the present methods include mRNA, long noncoding RNA, antisense transcripts, and pri-miRNAs. In some embodiments, the isolated RNA is mRNA. In a particular embodiment, mRNA is isolated by binding to a barcoded microparticle (e.g. a bead).

Methods of Identifying the Cell Type of Isolated Cells

As described above, the present methods contemplate sequencing a single cell transcriptome to determine the cell's identity (i.e., cell type) and/or to obtain information regarding genes and transgene expressed in that particular cell. Ultimately, the sequence information may be collected in a library, which can be used not only to identify the cell, but to determine which candidate regulatory element enabled expression of the transgene in the particular cell, as well as to quantify the level of transgene expression in the cell.

In some embodiments, the disclosure provides for methods incorporating any method which allows for the isolation of RNA from a single cell or single nucleus. In some embodiments, the disclosure provides for methods incorporating any method that allows for the analysis of mRNA transcripts while preserving information regarding the transcript's cell of origin. In some embodiments, the disclosure provides for methods incorporating any method which allows for the identification of a cell expressing the transgene operably linked to a candidate regulatory element. In one example, single cells can be identified by use of Droplet-Sequencing (also known as “Drop-Sequence” or “Drop-Seq”) methods. Drop-Sequence methods provide a high-throughput single-cell RNA-Seq and/or targeted nucleic acid profiling (e.g., sequencing, quantitative reverse transcription polymerase chain reaction, and the like) in which the RNAs from different cells are tagged individually using uniquely barcoded polynucleotides, allowing a single library to be created while retaining the cell identity of each sequenced mRNA. In some embodiments, a combination of molecular barcoding and emulsion-based microfluidics is used to isolate, lyse, barcode, and prepare nucleic acids from individual cells in a high-throughput manner.

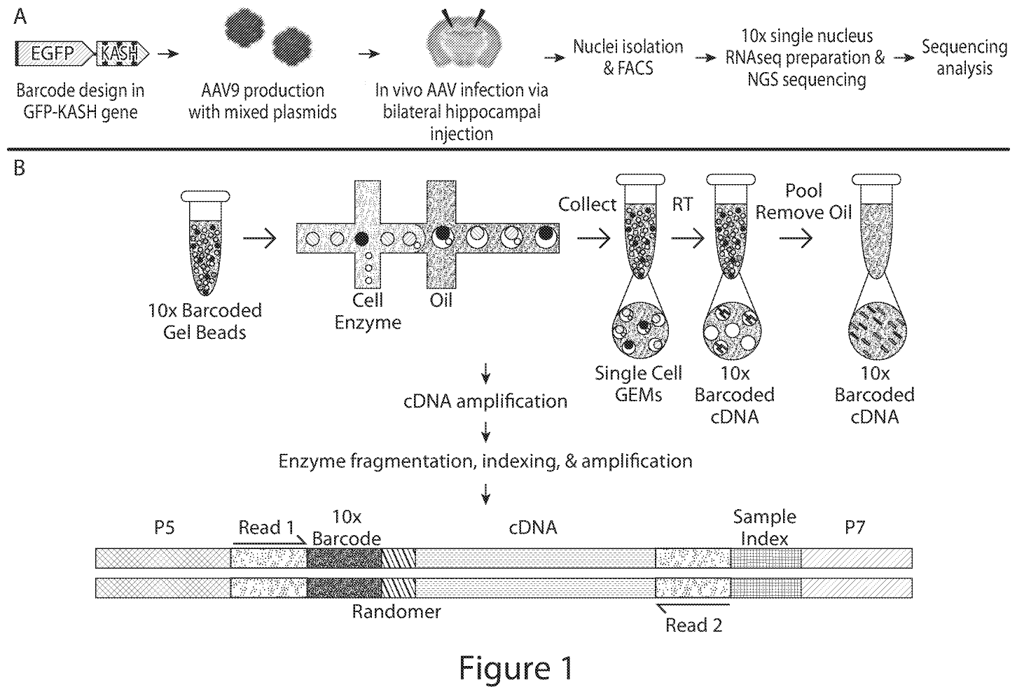

In the Drop-Sequence method, specially designed microparticles (e.g., beads) connected to uniquely barcoded polynucleotides are used for cell identification. As shown in FIG. 1, a single microparticle (bead) containing a large number of uniquely barcoded polynucleotides may be introduced into an individual emulsion droplet together with a single cell (or a single nucleus). In some embodiments, the barcoded polynucleotides are covalently attached to a microparticle (e.g., bead) (from 5′ to 3′, yielding free 3′ ends available for enzymatic priming) via a flexible multi-atom linker to form a barcoded capture bead. In some embodiments, the barcoded polynucleotides are covalently attached to a microparticle (e.g., bead) from 5′ to 3′, (yielding free 3′ ends available for enzymatic priming) via a flexible multi-atom linker to form a barcoded capture bead.

In some embodiments, any of the microparticles (e.g., beads) disclosed herein is connected to a polynucleotide molecule (referred to herein as a “microparticle polynucleotide”). In some embodiments, the microparticle polynucleotide comprises a constant sequence for use as a priming site for downstream PCR and sequencing. In some embodiments, the microparticle polynucleotide comprises a barcode sequence (a “cell barcode”) unique to the microparticle (e.g., bead), but that is common to all of the microparticle polynucleotides connected to the microparticle. In some embodiments, the microparticle polynucleotide comprises a Unique Molecular Identifier (UMI) nucleotide sequence which is unique to each microparticle polynucleotide. For example, if a microparticle comprises two or more microparticle polynucleotides, each microparticle polynucleotide on that microparticle would comprise a different UMI sequence. In some embodiments, the UMI may be used to identify PCR duplicates. In some embodiments, the microparticle polynucleotide comprises an oligo-dT sequence. In some embodiments, the oligo-dT sequence may be used to capture polyadenylated mRNAs (e.g., via hybridization with the polyA sequence of an mRNA) and/or priming reverse transcription.

In some embodiments, any of the microparticle polynucleotide molecules disclosed herein interacts with any of the nucleic acid molecules disclosed herein. In some embodiments, the nucleic acid molecule that interacts with (e.g., is connected to) the microparticle is an RNA molecule transcribed from a DNA molecule. In some embodiments, the RNA molecule comprises a transgene and a barcode sequence. In some embodiments, the DNA molecule comprises a regulatory element, wherein the barcode sequence in the RNA molecule correlates with the regulatory element in the DNA molecule. In some embodiments, the nucleic acid molecule comprises a polyA tail and the microparticle polynucleotide molecule comprises an oligo-dT sequence, and the polyA tail of the nucleic acid molecule hybridizes to the oligo-dT sequence of the microparticle polynucleotide.

In some embodiments, each microparticle polynucleotide molecule comprises four distinct regions: (1) a constant sequence for use as a priming site for downstream PCR and sequencing (identical on all microparticle polynucleotide molecules across all microparticles); (2) a “cell barcode” which is identical across all the microparticle polynucleotide molecules on any one microparticle, but different from the cell barcodes on other microparticles (i.e., a cell barcode is unique to a particular microparticle); (3) a Unique Molecular Identifier (UMI) which is different on each microparticle polynucleotide molecule, and is used to identify PCR duplicates; and (4) an oligo-dT sequence which is used for capturing polyadenylated mRNAs and priming reverse transcription.

As noted above, emulsion droplets (aqueous droplets that are surrounded by an immiscible carrier fluid) created by microfluidic devices can be used to co-encapsulate a cell (or a nucleus) with a barcoded microparticle. In some embodiments, the cell (or nucleus) is lysed within the droplet, and the mRNA (transcriptome) from the lysed cell or nucleus hybridizes to the numerous microparticle polynucleotide molecules (e.g., on the oligo-dT region of the microparticle polynucleotide molecule) of the microparticle (e.g., bead). See, e.g., FIG. 1. As described herein, in particular embodiments, the microparticle is uniquely barcoded so that each droplet and its contents are distinguishable. The methods disclosed herein contemplate single-cell approaches using any microparticle type (e.g. 10× Genomics Chromium Single Cell Gene Expression Assays). See, e.g., U.S. Published Application No. 20180030515 and Macosko et al., 2015, “Highly Parallel Genome-wide Expression Profiling of Individual Cells Using Nanoliter Droplets” Cell 161, 1202-1214; and Klein et al., 2015, “Droplet Barcoding for Single-Cell Transcriptomics Applied to Embryonic Stem Cells” Cell 161, 1187-1201, which are each incorporated herein by reference in their entirety. Other techniques that may be utilized to identify a cell that expresses a transgene include, for example, CEL-seq2/C1, MARS-seq, SCRB-seq, Smar-seq/C1, and/or Smart-seq2. See, e.g., Ziegenhain, et al., 2017, Molecular Cell, 65:631-643.

Single-Cell Transcriptome Sequencing

In some embodiments, the RNA from a lysed cell or nucleus, as discussed above, may be sequenced using any of the sequencing methods disclosed herein and the sequence information is collected to generate a sequence library. In some embodiments, the disclosure provides for methods incorporating any method which allows for the sequencing of a cell's transcriptome. Various methods for generating a sequence library are known in the art, and the methods are tailored to the particular high-throughput platform being used. In some embodiments, in mRNA analysis the 3′ polyadenylated (poly(A)) tail is targeted in order to ensure that coding RNA is separated from noncoding RNA. In the Drop-sequence method described herein, the barcoded microparticle polynucleotide molecules hybridize to the mRNAs. See, e.g., FIG. 1. In some embodiments, following capture of the mRNA on the barcoded microparticle, a reverse transcription (RT) reaction is performed to convert each cell's mRNA into a first strand cDNA that is both uniquely barcoded and covalently linked to the mRNA microparticle. Subsequently, in some embodiments, a universal primer via a template switching reaction is used to introduce a PCR handle downstream of the synthesized cDNA. In some embodiments, each of the cDNAs can then be amplified using PCR, quantified, and sequenced in parallel using a high-throughput platform such as next generation sequencing (NGS) to create data sets. PCR methods are well-known in the art. See, e.g., Dieffenbach and Dveksler, PCR Primer, a Laboratory Manual, Cold Spring Harbor Press, Plainview, N.Y. [1995]. NGS methods, such as the Illumina/Solexa™ platform and the NovaSeq™ platform are known to those of skill in the art.

Once sequencing is complete, the raw sequence data may, in some embodiments, undergo additional analysis. In some embodiments, conventional library preparation protocols can be used to prepare an RNA-Seq library. In some embodiments, a generalized data analysis pipeline for NGS data may be utilized. In some embodiments, a generalized data analysis pipeline for NGS data may include, but is not limited to, pre-processing the data to remove adapter sequences and low-quality reads, mapping of the data to a reference genome or de novo alignment of the sequence reads, and analysis of the compiled sequences. For example, in some embodiments, sequences can be aligned to a particular human transcriptome, and their “cell” barcode sequence information can be extracted in order to identify which mRNAs came from which cells. In some embodiments, sequences can be aligned to a particular human transcriptome, and their “UMI” barcode sequence information can be extracted in order to identify the abundance of a particular transcript in a particular cell. In some embodiments, sequences can be aligned to a particular human transcriptome, and their “cell” and “UMI” barcode sequence information can be extracted in order to identify which mRNAs came from which cells and the abundance of a particular transcript in a particular cell. Analysis of the sequences can include a wide variety of bioinformatic assessments, including, but not limited to, assessment for genetic variants calling for detection of small nucleotide polymorphisms (SNPs), detection of novel genes, identification of transgene insertion sites, determination of cell type expressing the transgene, identification of the candidate regulatory element involved with the expression of the transgene, and/or assessment of gene (e.g., transgene) transcript expression levels. In some embodiments, through a single sequencing run, tens of thousands (or more) of distinguishable transcriptomes can be simultaneously obtained.

Analysis of Single Cell Expression Profiles

In some embodiments, the disclosure provides for methods of assaying heterogeneous cell populations to identify a candidate regulatory element that provides selective expression in a given cell type. In some embodiments, the disclosure provides for methods incorporating any method which allows for the identification a cell which selectively expresses a transgene operably linked to a candidate regulatory element. In some embodiments, the cell may be within a heterogenous cell population. The heterogeneous cell populations may comprise not only cells of different types (e.g., cells of different lineages, cells of different differentiation status, and/or cells obtained from one or more tissue source throughout the body), but also cells in various cell cycle stages. In some embodiments, the transcriptome measurements from these heterogeneous cell populations may undergo a variety of bioinformatics assessments.

In some embodiments, raw sequence data can be aligned to a reference genome, providing a count of the number of reads associated with each gene. In some embodiments, raw sequence data can be aligned with sequence data in one or more molecular atlases of gene expression for known cell types or novel cell types. In some embodiments, the read count is determined by quantifying the number of transcripts using the UMI barcode to identify and remove transcripts which have been included due to PCR amplification bias. The data is normalized to account for cell to cell variation in the efficiencies of the cDNA library formation and sequencing. Numerous normalization methods are known in the art. See, e.g., Risso et al., 2018, “A General and Flexible Method for Signal Extraction from Single-Cell RNA-Seq Data” Nat. Comm. 9:284; 1-17 incorporated herein by reference in its entirety. In some embodiments, the cells or genes can be clustered to form subgroups based on their transcriptomic profile, allowing for the identification of cell subtypes or covarying genes, respectively. In some embodiments, various analyses such as principal component analysis (PCA) or t-SNE can be used to simplify the data for visualization and pattern detection by transforming cells from a high to a lower dimensional space. In some embodiments, representative cell markers (i.e., literature-derived canonical biomarkers) can be mapped onto each cluster in order to identify specific cell populations.

In some embodiments, if the cells express a barcoded transgene (e.g., a transgene encoding EGFP-KASH) operably linked to a candidate regulatory element, a comparative analysis of each transgene barcode can be performed to evaluate the effect that a given candidate regulatory element has on transgene expression in a particular cell type, as described herein. For example, the magnitude of expression of a particular transgene operably linked to a particular regulatory element can be evaluated. In some embodiments, the magnitude of expression (e.g., the level of decrease or increase of expression) of a particular transgene operably linked to a candidate regulatory element can be compared to the expression level of the same transgene operably linked to a different candidate regulatory element. In some embodiments, the magnitude of expression (e.g., the level of decrease or increase of expression) in one cell type of a particular transgene operably linked to a candidate regulatory element can be compared to the expression level of the same transgene linked to the same candidate regulatory element in a different cell type. Additionally, in some embodiments, it is further contemplated that comparisons can be made to compare the expression of a transgene operably linked to a candidate regulatory element amongst various cell types. In this way, the cell type specificity of the regulatory element and the magnitude of expression from a transgene operably linked to the regulatory element can be determined.

Determining Selective Expression Provided by a Regulatory Element

In some embodiments, the methods of the present disclosure include various methods, e.g., for isolating RNA from cells expressing the reporter transgene, sequencing the transcript of interest, measuring and/or detecting expression of the transgene, identifying the regulatory element that provides expression of the transgene in a cell type of interest, etc. The present methods can be used to identify and select a regulatory element suitable for expressing any transgene of interest in a target cell type based on the selectivity of expression of a transgene in the target cell type. In some embodiments the selectivity of expression of the transgene is a determination of whether the transgene is expressed in the target cell type as opposed to a non-target cell type. In some embodiments, the selectivity of expression of the transgene is a determination of whether the transgene is expressed at a greater level in the target cell type as opposed to a non-target cell type. In some embodiments, the selectivity of expression of the transgene is a determination of whether the transgene is expressed at a lower level in the target cell type as opposed to a non-target cell type.

In some embodiments, the present method can be used to identify a regulatory element that provides selective expression in any cell type of interest. In some embodiments, the cell type of interest is a muscle cell, a neuronal cell, an epithelial cell, or a connective tissue cell or various subpopulations thereof. In some embodiments, the muscle cell is a cardiomyocyte, skeletal muscle cell, cardiac muscle cell, or smooth muscle cell. In some embodiments, the epithelial cell is a squamous epithelial cell, a cuboidal epithelial cell, or a columnar epithelial cell. In some embodiments, the neuronal cell is a neuron or glial cell. In some embodiments, the connective tissue cell is a fibroblast, adipocyte, macrophage, mast cell, or plasma cell. In some embodiments, the cell of interest is a blood cell. In some embodiments, the cell of interest is a stem cell. In some embodiments, the cell of interest is a tumor cell (e.g., a cancer cell). In some embodiments, the cell type of interest is a eukaryotic cell such as a mammalian cell, which include, but are not limited to, cells from: humans, non-human primates (such as apes, chimpanzees, monkeys, and orangutans), domesticated animals, including dogs and cats, as well as livestock such as horses, cattle, pigs, sheep, and goats, or other mammalian species including, without limitation, mice, rats, guinea pigs, rabbits, hamsters, and the like. In some embodiments, the cell type of interest includes “transformants” and “transformed cells,” which include the primary transformed cell and progeny derived therefrom without regard to the number of passages.

In a simple scenario, a given candidate regulatory element (“regulatory element A”) may be determined to drive expression of a transgene to a higher level in a particular cell type than another regulatory element (“regulatory element B”) in the same cell type. In such a scenario, regulatory element A would be deemed to be more selective as compared to regulatory element B in enabling expression of the transgene in the particular cell type. In another simple scenario, a given regulatory element A may be determined to drive expression of a transgene to a lower level in a particular cell type than another regulatory element B in the same cell type. In some cases, a regulatory element A may enable wide-spread expression of the transgene across many different cell types of a given tissue (e.g., neuronal tissue). In some embodiments, a regulatory element B may enable expression of the transgene in a discrete population of the target cells of the given tissue (i.e., the regulatory element B provides a higher ratio of target cells expressing the transgene vs. total number of cells expressing the transgene). In this scenario, the regulatory element B would be deemed to be more selective as compared to regulatory element A in enabling expression of the transgene in a more limited subset of cell type(s), which may be beneficial for, e.g., reducing off-target events. In the above simplified example scenarios, neither comparison for determining selectivity is mutually exclusive.

In some embodiments, multiple comparisons can be considered for a particular use of a regulatory element and/or to achieve a specific therapeutic purpose. In some embodiments, a regulatory element suitable for a specific therapeutic purpose need not provide the highest or lowest level of expression in a given cell type. As detailed herein, selectivity of expression driven by a candidate regulatory element can be measured and determined in a number of ways.

In one aspect, the present methods can be used to screen and identify, from a pool of candidate regulatory elements operably linked to a transgene (e.g., a reporter gene), the regulatory element(s) that allows any detectable expression of the transgene in a cell type of interest. That is, any detectable expression of the transgene operably linked to a given candidate regulatory element in a cell type of interest indicates that the regulatory element can be used in the cell type of interest to drive expression of any transgene. By way of example, a regulatory element that has been identified to drive expression of a transgene (e.g., a reporter gene) in PV cells indicates that the identified regulatory element can be used in PV cells to drive expression of a transgene of interest. In some embodiments, the expression level of the transgene need not be compared to a reference expression level; any detectable level of expression of a transgene operably linked to a regulatory element indicates that the regulatory element provides selective expression in a given cell type. Thus, in some embodiments, the identified regulatory element selectively drives expression of the transgene in one cell type as compared to another cell type (in which no or low expression is detected). In some embodiments, the identified regulatory element selectively drives expression of the transgene in one cell type as compared to another candidate regulatory element (which did not drive expression of the transgene in the same cell type).

In some aspects, the methods described herein can be used to screen and identify, from a pool of candidate regulatory elements operably linked to a transgene (e.g., a reporter gene), the regulatory element(s) that allows selective (e.g., increased or decreased) expression of the transgene in a cell type of interest as compared to a reference expression level of the transgene in the same cell type. In some embodiments, the reference expression level of the transgene is the level of transgene expression provided by a control regulatory element. The skilled worker is aware of numerous exemplary control regulatory elements in the art (e.g., CBA). In some embodiments, the control regulatory element is naturally occurring regulatory element (e.g., CBA). In some embodiments, the reference expression level of the transgene is the level of transgene expression provided by another candidate regulatory element in the same cell type. In some embodiments, the reference expression level of the transgene is the level of transgene expression provided by a pan-cellular regulatory element in the same cell type. Examples of pan-cellular regulatory element include, e.g., cytomegalovirus major immediate-early promoter (CMV), chicken β-actin promoter (CBA), CMV early enhancer/CBA promoter (CAG), elongation factor-1α promoter (EF1α), simian virus 40 promoter (SV40), phosphoglycerate kinase promoter (PGK), and the polyubiquitin C gene promoter (UBC), and as described herein. By way of example, selectivity of a candidate regulatory element in a cell type of interest can be determined by comparing the level of expression provided by the regulatory element in the cell type to the level of expression driven by one or more different candidate regulatory elements in the same cell type. In some embodiments, the regulatory element provides selective expression that is at least 1.2 fold, at least 1.4 fold, at least 1.6 fold, at least 1.8 fold, at least 2 fold, at least 3 fold at least 4 fold, at least 5 fold, at least 6 fold, at least 7 fold, at least 8 fold, at least 9 fold, at least 10 fold, at least 12 fold, at least 14 fold, at least 16 fold, at least 18 fold, at least 20 fold greater or less as compared to a reference expression level (e.g., level of transgene expression provided by another candidate regulatory element; level of transgene expression provided by a pan-cellular regulatory element) in the same cell type. In some embodiments, the regulatory element provides selective expression that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 250%, at least 300%, at least 350%, at least 400%, at least 450%, or at least 500% greater as compared to a reference expression level (e.g., level of transgene expression provided by another candidate regulatory element; level of transgene expression provided by a pan-cellular regulatory element) in the same cell type. In some embodiments, the regulatory element provides selective expression that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% less as compared to a reference expression level (e.g., level of transgene expression provided by another candidate regulatory element; level of transgene expression provided by a pan-cellular regulatory element) in the same cell type. In some embodiments, the regulatory element provides selective expression that is about 1.5 times, about 2 times, about 2.5 times, about 3 times, about 3.5 times, about 4 times, about 4.5 times, about 5 times, about 5.5 times, about 6 times, about 6.5 times about 7 times, about 7.5 times, about 8 times, about 8.5 times, about 9 times, about 9.5 times, or about 10 times greater as compared to a reference expression level (e.g., level of transgene expression provided by another candidate regulatory element; level of transgene expression provided by a pan-cellular regulatory element) in the same cell type. In some embodiments, the regulatory element provides selective expression that is about 1.5 times, about 2 times, about 2.5 times, about 3 times, about 3.5 times, about 4 times, about 4.5 times, about 5 times, about 5.5 times, about 6 times, about 6.5 times about 7 times, about 7.5 times, about 8 times, about 8.5 times, about 9 times, about 9.5 times, or about 10 times less as compared to a reference expression level (e.g., level of transgene expression provided by another candidate regulatory element; level of transgene expression provided by a pan-cellular regulatory element) in the same cell type.

In some aspects, any of the methods described herein can be used to screen and identify, from a pool of candidate regulatory elements operably linked to a transgene (e.g., a reporter gene), the selective (e.g., increased or decreased) expression of the transgene operably linked to a regulatory element in one cell type as compared to the expression level of the same transgene operably linked to the same regulatory element in one or more different cell types (the reference expression level). By way of example, selectivity of a candidate regulatory element in a cell type of interest can be determined by comparing the level of expression provided by the regulatory element in the cell type to the level of expression provided by the same regulatory element in one or more different cell types. In some embodiments, the regulatory element provides selective expression that is at least 1.2 fold, at least 1.4 fold, at least 1.6 fold, at least 1.8 fold, at least 2 fold, at least 3 fold at least 4 fold, at least 5 fold, at least 6 fold, at least 7 fold, at least 8 fold, at least 9 fold, at least 10 fold, at least 12 fold, at least 14 fold, at least 16 fold, at least 18 fold, at least 20 fold greater as compared to a reference expression level (e.g., level of transgene expression provided by the same regulatory element in one or more different cell types). In some embodiments, the regulatory element provides selective expression that is at least 1.2 fold, at least 1.4 fold, at least 1.6 fold, at least 1.8 fold, at least 2 fold, at least 3 fold at least 4 fold, at least 5 fold, at least 6 fold, at least 7 fold, at least 8 fold, at least 9 fold, at least 10 fold, at least 12 fold, at least 14 fold, at least 16 fold, at least 18 fold, at least 20 fold less as compared to a reference expression level (e.g., level of transgene expression provided by the same regulatory element in one or more different cell types). In some embodiments, the regulatory element provides selective expression that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 250%, at least 300%, at least 350%, at least 400%, at least 450%, or at least 500% greater as compared to a reference expression level (e.g., level of transgene expression provided by the same regulatory element in one or more different cell types). In some embodiments, the regulatory element provides selective expression that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% less as compared to a reference expression level (e.g., level of transgene expression provided by the same regulatory element in one or more different cell types). In some embodiments, the regulatory element provides selective expression that is about 1.5 times, about 2 times, about 2.5 times, about 3 times, about 3.5 times, about 4 times, about 4.5 times, about 5 times, about 5.5 times, about 6 times, about 6.5 times about 7 times, about 7.5 times, about 8 times, about 8.5 times, about 9 times, about 9.5 times, or about 10 times greater as compared to a reference expression level (e.g., level of transgene expression provided by the same regulatory element in one or more different cell types). In some embodiments, the regulatory element provides selective expression that is about 1.5 times, about 2 times, about 2.5 times, about 3 times, about 3.5 times, about 4 times, about 4.5 times, about 5 times, about 5.5 times, about 6 times, about 6.5 times about 7 times, about 7.5 times, about 8 times, about 8.5 times, about 9 times, about 9.5 times, or about 10 times less as compared to a reference expression level (e.g., level of transgene expression provided by the same regulatory element in one or more different cell types).

In some embodiments, selectivity of transgene expression operably linked to a regulatory element can be determined by methods that measure a ratio of a particular cell type of interest (a hypothetical cell type of interest “Cell X”) that expresses a transgene in a population of cells (e.g., in a tissue). In some embodiments, determination of a ratio does not include measuring a level or magnitude of transgene expression; rather, in such embodiments, any detectable expression in the cell contributes to the ratio. In some embodiments, selectivity of transgene expression operably linked to a candidate regulatory element can be measured by comparing the number of Cell X cells that express a pre-determined threshold level (e.g., a detectable level) of the transgene in a population of cells (e.g., in a tissue) to the total number of cells that express the transgene operably linked to the same regulatory element. In some embodiments, this “ratio” is calculated as being the number of transgene-expressing Cell X cells vs. the total number of transgene-expressing cells in the cell population (Cell X+non-Cell X cells), wherein the transgene is operably linked to the same regulatory element in all of the cells in the cell population. By way of example, selective expression of a transgene (e.g., a transgene encoding GFP) operably linked to a regulatory element in GABAergic neurons, such as PV neurons as compared to other non-PV cells in neuronal tissue can be measured by comparing the number of PV cells that express a detectable level of the transgene (e.g., express the GFP transgene) to the total number of cells in the neuronal tissue that express GFP under the control of the same regulatory element A (i.e., the ratio of PV vs. total cells (PV+non-PV cells) expressing GFP). Such measurement, detection, and quantification can be done either in vivo or in vitro, according to the assay methods described herein. For example, using the analysis methods detailed herein, cells expressing GFP can be separated and isolated, the identity of each isolated cell can be determined (e.g., PV neuron versus non-PV cells), and the number of GFP-expressing PV neurons under the control of a candidate regulatory element and GFP-expressing non-PV neurons under the control of the same regulatory element can be quantified. In some embodiments, the higher the number of Cell X cells that expresses the transgene operably linked to a regulatory element vs. the total cells that express the transgene operably linked to the same regulatory element (i.e., the higher the ratio), the more selective the regulatory element is for Cell X.

In some embodiments, selectivity of a regulatory element in a cell type can be determined or validated using an immunohistochemistry-based colocalization assay. In some embodiments, the assay entails using: a) a transgene (e.g., a transgene encoding GFP) operably linked to regulatory element to measure transgene expression and, b) a binding agent (e.g., an antibody) that identifies a marker that is specific to a target cell type, wherein the binding agent is linked to a detectable label. For example, in some embodiments, selectivity for a cell type can be determined or validated using an immunohistochemistry-based colocalization assay using: a) a transgene (e.g., a transgene encoding GFP) operably linked to regulatory element to measure transgene expression and, b) an antibody that identifies the cell type of interest (e.g., an anti-PV antibody that interacts specifically with PV neurons) linked to a second fluorescence label (e.g., red fluorescent protein). Selectivity of gene expression in a cell type is measured as percentage of GFP positive cells (e.g., total cells) that are also positive for the cell type (e.g., PV cells). In such an assay, the positive cell types of interest that are also GFP positive are indicated by the colocalization of both fluorescence signals, i.e., an overlap of the red and green fluorescence. Such measurement, analysis, and/or detection can be done by eye inspection or by a computer.

In some embodiments, the “ratio” as described herein can be calculated by dividing the number of Cell X cells expressing a transgene operably linked to a candidate regulatory element by the total number of cells that express the transgene operably linked to the same regulatory element (i.e., Cell X and non-Cell X cells), and multiplying by 100 to convert into a percentage. In some embodiments, a regulatory element A is selective for Cell X if about 35%, 40%, 45%, 50%, 55%, 60%, 65%, 70%, 75%, 80%, 85%, 90%, 91%, 92%, 93%, 94%, 95%, 96%, 97%, 98%, 99%, or greater than about 99% of the total number of cells expressing the transgene operably linked to regulatory element A are Cell X cells.

In some embodiments, the ratio (or percentage) as described above can be determined for Cell X cells using a regulatory element and comparing it to a ratio (or percentage) determined for Cell X cells using one or more different regulatory elements. For example, in some embodiments, a regulatory element is selective for expression in Cell X when the percentage of Cell X cells (e.g., Cell X cells/total cells×100) expressing the transgene is at a higher percentage than the percentage of Cell X cells expressing the same transgene when operably linked to a different regulatory element. In some embodiments, the different regulatory element is a reference regulatory element. In some embodiments, the different regulatory element is a pan-cellular regulatory element, e.g., cytomegalovirus major immediate-early promoter (CMV), chicken β-actin promoter (CBA), CMV early enhancer/CBA promoter (CAG), elongation factor-1α promoter (EF1α), simian virus 40 promoter (SV40), phosphoglycerate kinase promoter (PGK), and the polyubiquitin C gene promoter (UBC), and as described herein. In some embodiments, a regulatory element provides selective expression in Cell X when the percentage of Cell X cells (e.g., Cell X cells/total cells×100) expressing the transgene is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 250%, at least 300%, at least 350%, at least 400%, at least 450%, or at least 500% higher, or at least 1-5%, 5%-10%, 10-15%, 15-20%, 20-25%, 25-30%, 30-35%, 35-40%, 40-45%, 45-50%, 50-55%, 55-60%, 65-70%, 70-75%, 75-80%, 80-85%, 85-90%, 90-95%, 100-125%, 125-150%, 150-200%, 200-250%, 250-300%, 300-350%, 350-400%, 400-450%, or 450-500% higher than the percentage of Cell X cells expressing the same transgene when operably linked to a different regulatory element. In some embodiments, a regulatory element provides selective expression in Cell X when the percentage of Cell X cells (e.g., Cell X cells/total cells×100) expressing the transgene is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% less, or at least 1-5%, 5%-10%, 10-15%, 15-20%, 20-25%, 25-30%, 30-35%, 35-40%, 40-45%, 45-50%, 50-55%, 55-60%, 65-70%, 70-75%, 75-80%, 80-85%, 85-90%, or 90-95% less than the percentage of Cell X cells expressing the same transgene when operably linked to a different regulatory element. In some embodiments, a regulatory element provides selective expression in Cell X when the percentage of Cell X cells (e.g., Cell X cells/total cells×100) expressing the transgene is at least 1.5 fold, at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, or at least 50 fold higher as compared to the percentage of Cell X cells expressing the same transgene when operably linked to a different regulatory element. In some embodiments, a regulatory element provides selective expression in Cell X when the percentage of Cell X cells (e.g., Cell X cells/total cells×100) expressing the transgene is at least 1.5 fold, at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, or at least 50 fold lower as compared to the percentage of Cell X cells expressing the same transgene when operably linked to a different regulatory element. In some embodiments, a regulatory element provides selective expression in Cell X when the percentage of Cell X cells (e.g., Cell X cells/total cells×100) expressing the transgene is at a level that is at least 1.1, 1.2, 1.3, 1.4, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, or 100 times higher than the percentage of Cell X cells expressing the transgene when operably linked to a different regulatory element.

In some embodiments, a regulatory element that provides selective expression in Cell X also has high levels of activity. In certain embodiments, the regulatory element that provides selective expression in Cell X increases expression of a transgene in Cell X cells by at least 2, 5, 10, 15, 20, 30, 40, 50, 60, 70, 80, 90, 100, or more fold as compared to the level of expression of the same construct in Cell X cells without the regulatory element or with a different regulatory element (a reference regulatory element). In some embodiments, a regulatory element that provides selective expression in Cell X increases gene expression by at least 1.5%, 2%, 5%, 10%, 15%, 20%, 50%, 55%, 60%, 65%, 70%, 75%, 80%, 85%, 90%, 95%, or 100% as compared to the level of expression of the same construct in Cell X cells without the regulatory element or with a different regulatory element (a reference regulatory element). In some embodiments, a regulatory element that provides selective expression in Cell X increases gene expression in Cell X cells by at least 1.5%, 2%, 5%, 10%, 15%, 20%, 50%, 55%, 60%, 65%, 70%, 75%, 80%, 85%, 90%, 95%, or 100% as compared to the level of expression of the same construct in a cell type different from Cell X. In some embodiments, a regulatory element increases transgene expression in Cell X cells by at least 1.5%, 2%, 5%10%, 15%, 20%, 50%, 55%, 60%, 65%, 70%, 75%, 80%, 85%, 90%, 95%, or 100% as compared to the amount of increase in expression in a different cell expressing the same transgene operably linked to the same regulatory element. In some embodiments, a regulatory element increases transgene expression in Cell X cells by at least 1.5%, 2%, 5%, 10%, 15%, 20%, 50%, 55%, 60%, 65%, 70%, 75%, 80%, 85%, 90%, 95%, or 100% as compared to the amount of increase in expression in Cell X cells expressing the same transgene operably linked to a different regulatory element (e.g., a reference regulatory element or a pan cellular regulatory element).

Generally, an increase or decrease in expression can occur at the transcriptional or posttranscriptional level, and either the transcriptional or posttranscriptional product can be measured. For example, at the transcriptional level, a regulatory element can increase expression by recruiting transcription factors, and/or RNA polymerase, increasing initiation of transcription or recruiting DNA and/or histone modifications that increase the level of transcription. An increase or decrease in expression can be detected by measuring an increase or decrease in the amount of RNA transcripts that are representative of the transgene. At the posttranscriptional level, a regulatory element can increase expression by increasing the amount of or rate at which RNA that is translated into protein. This can be achieved through various mechanisms, for example, by increasing the stability of the mRNA or increasing recruitment and assembly of proteins required for translation. Such increase or decrease of protein expression can be detected by measuring the amount of protein expressed that is representative of the transgene. The amount of protein produced can be measured directly, for example by an enzyme linked immunosorbent assay (ELISA), or indirectly, for example, by a functional assay.

The selectivity of various REs identified using the methods described above can be further tested and validated for selective gene expression in specific cell types. For instance, REs can be tested for selective gene expression in GABAergic neurons such as PV, SST, or VIP neurons using immunohistochemical methods. GABAergic neurons can be identified by markers such as the expression of glutamic acid decarboxylase 2 (GAD2), GAD1, NKX2.1, DLX1, DLX5, SST, PV and VIP. Alternatively, REs can be tested for selective gene expression in other cell types such as excitatory neurons, dopaminergic neurons, microglia, motor neurons, vascular cells, non-GABAergic neurons or other CNS cells, epithelial cells, cardiomyocytes, or hepatocytes, or any other cell type in the body. Selectivity of expression driven by a regulatory element in a cell or cell type of interest can be measured in a number of ways. Selectivity of gene expression in a target cell type over non-target cell types can be measured by comparing the number of target cells that express a detectable level of a transcript from a gene that is operably linked to one or more regulatory elements to the total number of cells that express the gene. Such measurement, detection, and quantification can be done either in vivo or in vitro.

In some instances, selectivity for a specific cell type can be determined using a co-localization assay. In some cases, the co-localization assay is based on immunohistochemistry. In some cases, a detectable reporter gene is used as a transgene to allow the detection and/or measurement of gene expression in the cell type of interest. In some cases, a detectable marker, e.g., a fluorescent marker or an antibody, which specifically labels the target cell is used to detect and/or measure the target cells. In some cases, a co-localization assay employs imaging, e.g., fluorescent imaging, to determine the overlap between different fluorescent labels, e.g., overlap between a fluorescence signal indicative of a target cell and another fluorescence signal indicative of gene expression. In some cases, fluorescent labels used for a co-localization assay include a red fluorescent protein (RFP), such as a tdTomato reporter gene, and a green fluorescent reporter protein, such as eGFP.

In some instances, a gene operably linked to one or more regulatory elements is a fluorescent protein, e.g., eGFP or RFP, wherein expression of the transgene provides a detectable signal. In some cases, tissue is stained for eGFP or fluorescence from eGFP is detected directly using a fluorescence microscope. A second fluorescent marker or reporter gene having a different fluorescence or detectable signal can be used to indicate the target cells, such as an antibody that identifies the target cells. For example, an anti-PV antibody that interacts specifically with PV neurons can be used to yield a detectable signal that is distinguishable from the fluorescence used to measure gene expression, such as a red fluorescence or a red stain. Thus, in an example wherein eGFP is a transgene operably linked to one or more regulatory elements that drive selective expression in PV neurons, and wherein the PV neurons are labeled with an anti-PV antibody, selectivity of gene expression in PV cells is measured as percentage of eGFP+ cells that are also PV+. In such assay, PV+ cells that are also eGFP+ are indicated by the overlap of both fluorescence signals, i.e., an overlap of the red and green fluorescence. Such measurement, analysis, and/or detection can be done by eye inspection or by a computer.

In some cases, one can also measure the proportion of a cell type of interest (or target cell type) that expresses a transgene as compared to the proportion of non-target cell types (or other cells) that express the transgene to assess the selectivity of one or more regulatory elements operably linked to the transgene. Similarly, selectivity of expression can also be measured by comparing the number of target cells that express a transgene operably linked to one or more regulatory elements to the total number of all cells that express the transgene. In both approaches, the higher the number of target cells that express the transgene, the more selective are the regulatory elements for the target cells. In some cases, the target cells are PV neurons.

### Alternative Applications of the Single Nucleus Multiplex Assay

In certain embodiments, the single nucleus multiplex assay described herein is used to measure AAV transduction in a cell of interest. In such embodiments, the multiplex assay can be used to measure transduction of a specific virus of interest into a cell of interest, such as a specific AAV serotype, a recombinant or engineered AAV, or a specific lentiviral strain. In certain embodiments, the multiplex assay is used to measure transduction of an AAV selected from the group consisting of: AAV1, AAV2, AAV3, AAV4, AAV5, AAV6, AAV7, AAV8, AAV9, AAV10, AAV11, AAV12, rh10, and hybrids thereof, avian AAV, bovine AAV, canine AAV, equine AAV, primate AAV, non-primate AAV, and ovine AAV, into a cell of interest. In certain embodiments, the single nucleus multiplex assay described herein is used to measure AAV transduction into a cell type of interest, such as a CNS cell (e.g., a neuron, or a glial cell such as an astrocyte), a non-CNS cell (e.g., excitatory neurons, dopaminergic neurons, microglia, motor neurons, vascular cells, non-GABAergic neurons, or other CNS cells), epithelial cells, cardiomyocytes, or hepatocytes. In particular embodiments, the single nucleus multiplex assay described herein is used to measure AAV transduction into a GABAergic neuron, which can be identified by markers such as the expression of glutamic acid decarboxylase 2 (GAD2), GAD1, NKX2.1, DLX1, DLX5, SST, PV and VIP.

In particular embodiments, the single nucleus multiplex assay of the invention is used to identify novel viral capsids or viral DNA sequences that increase transduction of the virus into a cell interest, by measuring an increase or decrease in viral transduction in a cell of interest. For instance, a library of novel viral capsid variants or viral DNA sequences can be screened to identify a capsid or DNA sequence that increases viral transduction (e.g., an AAV or lentivirus) into a cell of interest. In some cases, a capsid or DNA sequence increases viral transduction into a cell type of interest, such as a CNS cell (e.g., a neuron, or a glial cell such as an astrocyte), a non-CNS cell (e.g., excitatory neurons, dopaminergic neurons, microglia, motor neurons, vascular cells, non-GABAergic neurons, or other CNS cells), epithelial cells, cardiomyocytes, or hepatocytes. In particular cases, the single nucleus multiplex assay described herein is used to identify capsid or DNA sequences that increase AAV transduction into a GABAergic neuron, such as a GABAergic neuron that expresses glutamic acid decarboxylase 2 (GAD2), GAD1, NKX2.1, DLX1, DLX5, SST, PV or VIP.

In other embodiments, a library of novel viral capsid variants or viral DNA sequences can be screened to identify a viral capsid or viral DNA sequence that decreases or inhibits viral transduction (e.g., an AAV or lentivirus) into a cell of interest. For instance, a capsid or DNA sequence decreases or inhibit viral transduction into a cell type of interest, such as a CNS cell (e.g., a neuron, or a glial cell such as an astrocyte), a non-CNS cell (e.g., excitatory neurons, dopaminergic neurons, microglia, motor neurons, vascular cells, non-GABAergic neurons, or other CNS cells), epithelial cells, cardiomyocytes, or hepatocytes. In particular embodiments, the single nucleus multiplex assay described herein is used to identify capsid or DNA sequences that decrease or inhibit AAV transduction into a GABAergic neuron, such as a GABAergic neuron that expresses glutamic acid decarboxylase 2 (GAD2), GAD1, NKX2.1, DLX1, DLX5, SST, PV or VIP.

In another embodiment, the single nucleus multiplex assay of the invention is used to identify a factor that regulates translation of a transgene that is transduced into a cell of interest by a virus (e.g., AAV, lentivirus, HSV, etc.). For instance, a library of candidate factors is screened to identify a factor that increases or decreases translation of a transgene that is transduced into a cell of interest by a virus (e.g., AAV, lentivirus, HSV, etc.). In one embodiment, a factor increases or decreases translation of a transgene that is transduced into a cell of interest, such as a CNS cell (e.g., a neuron, or a glial cell such as an astrocyte), a non-CNS cell (e.g., excitatory neurons, dopaminergic neurons, microglia, motor neurons, vascular cells, non-GABAergic neurons, or other CNS cells), epithelial cells, cardiomyocytes, or hepatocytes. In particular embodiments, the single nucleus multiplex assay described herein is used to identify a factor that increases or decreases translation of a transgene that is transduced into a GABAergic neuron, such as a GABAergic neuron that expresses glutamic acid decarboxylase 2 (GAD2), GAD1, NKX2.1, DLX1, DLX5, SST, PV or VIP.

In another embodiment, the single nucleus multiplex assay of the invention is used to identify viral DNA sequences that facilitate viral (e.g., AAV) second strand synthesis in a cell of interest. For instance, a library of novel viral DNA sequences can be screened to identify a DNA sequence that increases or decreases AAV second strand synthesis in a cell of interest. For instance, a DNA sequence increases or decreases AAV second strand synthesis in a cell type of interest, such as a CNS cell (e.g., a neuron, or a glial cell such as an astrocyte), a non-CNS cell (e.g., excitatory neurons, dopaminergic neurons, microglia, motor neurons, vascular cells, non-GABAergic neurons, or other CNS cells), epithelial cells, cardiomyocytes, or hepatocytes. In particular embodiments, the single nucleus multiplex assay described herein is used to identify viral DNA sequences that increase or decrease AAV second strand synthesis in a GABAergic neuron, such as a GABAergic neuron that expresses glutamic acid decarboxylase 2 (GAD2), GAD1, NKX2.1, DLX1, DLX5, SST, PV or VIP.

In another embodiment, the single nucleus multiplex assay of the invention is used to measure gene expression in a cell of interest in response to a functional protein of interest, such as a functional protein effector. In such embodiment, a library of proteins can be added to one or more cells, and gene expression in response to each unique protein is measured in a cell of interest. The gene expression can be analyzed for therapeutic response, cell pathway signaling response, off-target gene regulation, immune response, etc., in response to one or more proteins from the library.

SEQUENCES
SEQ ID NO: 1
TCAACAGGGGGACACTTGGGAAAGAAGGATGGGGACAGAGCCGAGAGGAC
TGTTACACATTAGAGAAACATCAGTGACTGTGCCAGCTTTGGGGTAGACT
GCACAAAAGCCCTGAGGCAGCACAGGCAGGATCCAGTCTGCTGGTCCCAG
GAAGCTAACCGTCTCAGACAGAGCACAAAGCACCGAGACATGTGCCACAA
GGCTTGTGTAGAGAGGTCAGAGGACAGCGTACAGGTCCCAGAGATCAAAC
TCAACCTCACCAGGCTTGGCAGCAAGCCTTTACCAACCCACCCCCACCCC
ACCCACCCTGCACGCGCCCCTCTCCCCTCCCCATGGTCTCCCATGGCTAT
CTCACTTGGCCCTAAAATGTTTAAGGATGACACTGGCTGCTGAGTGGAAA
TGAGACAGCAGAAGTCAACAGTAGATTTTAGGAAAGCCAGAGAAAAAGGC
TTGTGCTGTTTTTAGAAAGCCAAGGGACAAGCTAAGATAGGGCCCAAGTA
ATGCTAGTATTTACATTTATCCACACAAAACGGACGGGCCTCCGCTGAAC
CAGTGAGGCCCCAGACGTGCGCATAAATAACCCCTGCGTGCTGCACCACC
TGGGGAGAGGGGGAGGACCACGGTAAATGGAGCGAGCGCATAGCAAAAGG
GACGCGGGGTCCTTTTCTCTGCCGGTGGCACTGGGTAGCTGTGGCCAGGT
GTGGTACTTTGATGGGGCCCAGGGCTGGAGCTCAAGGAAGCGTCGCAGGG
TCACAGATCTGGGGGAACCCCGGGGAAAAGCACTGAGGCAAAACCGCCGC
TCGTCTCCTACAATATATGGGAGGGGGAGGTTGAGTACGTTCTGGATTAC
TCATAAGACCTTTTTTTTTTCCTTCCGGGCGCAAAACCGTGAGCTGGATT
TATAATCGCCCTATAAAGCTCCAGAGGCGGTCAGGCACCTGCAGAGGAGC
CCCGCCGCTCCGCCGACTAGCTGCCCCCGCGAGCAACGGCCTCGTGATTT
CCCCGCCGATCCGGTCCCCGCCTCCCCACTCTGCCCCCGCCTACCCCGGA
GCCGTGCAGCCGCCTCTCCGAATCTCTCTCTTCTCCTGGCGCTCGCGTGC
GAGAGGGAACTAGCGAGAACGAGGAAGCAGCTGGAGGTGACGCCGGGCAG
ATTACGCCTGTCAGGGCCGAGCCGAGCGGATCGCTGGGCGCTGTGCAGAG
GAAAGGCGGGAGTGCCCGGCTCGCTGTCGCAGAGCCGAGGTGGGTAAGCT
AGCGACCACCTGGACTTCCCAGCGCCCAACCGTGGCTTTTCAGCCAGGTC
CTCTCCTCCCGCGGCTTCTCAACCAACCCCATCCCAGCGCCGGCCACCCA
ACCTCCCGAAATGAGTGCTTCCTGCCCCAGCAGCCGAAGGCGCTACTAGG
AACGGTAACCTGTTACTTTTCCAGGGGCCGTAGTCGACCCGCTGCCCGAG
TTGCTGTGCGACTGCGCGCGCGGGGCTAGAGTGCAAGGTGACTGTGGTTC
TTCTCTGGCCAAGTCCGAGGGAGAACGTAAAGATATGGGCCTTTTTCCCC
CTCTCACCTTGTCTCACCAAAGTCCCTAGTCCCCGGAGCAGTTAGCCTCT
TTCTTTCCAGGGAATTAGCCAGACACAACAACGGGAACCAGACACCGAAC
CAGACATGCCCGCCCCGTGCGCCCTCCCCGCTCGCTGCCTTTCCTCCCTC
TTGTCTCTCCAGAGCCGGATCTTCAAGGGGAGCCTCCGTGCCCCCGGCTG
CTCAGTCCCTCCGGTGTGCAGGACCCCGGAAGTCCTCCCCGCACAGCTCT
CGCTTCTCTTTGCAGCCTGTTTCTGCGCCGGACCAGTCGAGGACTCTGGA
CAGTAGAGGCCCCGGGACGACCGAGCTG
SEQ ID NO: 2
GAGGAGGAGGAGGAGACAGACAGCAGGATGCCCCACCTCGACAGCCCCGG
TTCATCACAACCGAGACGCTCCTTCCTCTCAAGGGTGATCAGGGCAGCTC
TACCGTTGCAGCTGCTTCTGCTGCTGCTGCTGCTCCTGGCCTGCCTGTTG
CCTGCTTCAGAGGATGACTACAGCTGCACCCAGGCCAACAACTTTGCCCG
ATCCTTCTACCCCATGCTGCGGTACACCAACGGGCCACCTCCCACCTAGG
ACTCAGCT
SEQ ID NO: 3
GAGGAGGAGGAGGAGACAGACAGCAGGATGCCCCACCTCGACAGCCCCGG
TAGCAGCCAACCGAGACGCTCCTTCCTCTCAAGGGTGATCAGGGCAGCTC
TACCGTTGCAGCTGCTTCTGCTGCTGCTGCTGCTCCTGGCCTGCCTGTTG
CCTGCCAGCGAGGATGACTACAGCTGCACCCAGGCCAACAACTTTGCCCG
ATCCTTCTACCCCATGCTGCGGTACACCAACGGGCCACCTCCCACCTAGC
TTACTAGC
SEQ ID NO: 4
GAGGAGGAGGAGGAGACAGACAGCAGGATGCCCCACCTCGACAGCCCCGG
CAGTAGTCAACCGAGACGCTCCTTCCTCTCAAGGGTGATCAGGGCAGCTC
TACCGTTGCAGCTGCTTCTGCTGCTGCTGCTGCTCCTGGCCTGCCTGTTG
CCCGCTAGTGAGGATGACTACAGCTGCACCCAGGCCAACAACTTTGCCCG
ATCCTTCTACCCCATGCTGCGGTACACCAACGGGCCACCTCCCACCTAGT
CAGGAATC
SEQ ID NO: 5
GAGGAGGAGGAGGAGACAGACAGCAGGATGCCCCACCTCGACAGCCCCGG
CTCGTCGCAACCGAGACGCTCCTTCCTCTCAAGGGTGATCAGGGCAGCTC
TACCGTTGCAGCTGCTTCTGCTGCTGCTGCTGCTCCTGGCCTGCCTGTTG
CCCGCCTCGGAGGATGACTACAGCTGCACCCAGGCCAACAACTTTGCCCG
ATCCTTCTACCCCATGCTGCGGTACACCAACGGGCCACCTCCCACCTAGA
GACAGGTA
SEQ ID NO: 6
GAGGAGGAGGAGGAGACAGACAGCAGGATGCCCCACCTCGACAGCCCCGG
ATCTTCTCAACCGAGACGCTCCTTCCTCTCAAGGGTGATCAGGGCAGCTC
TACCGTTGCAGCTGCTTCTGCTGCTGCTGCTGCTCCTGGCCTGCCTGTTG
CCAGCATCTGAGGATGACTACAGCTGCACCCAGGCCAACAACTTTGCCCG
ATCCTTCTACCCCATGCTGCGGTACACCAACGGGCCACCTCCCACCTAGG
ATTCTCAG
SEQ ID NO: 7
GAGGAGGAGGAGGAGACAGACAGCAGGATGCCCCACCTCGACAGCCCCGG
GTCCTCCCAACCGAGACGCTCCTTCCTCTCAAGGGTGATCAGGGCAGCTC
TACCGTTGCAGCTGCTTCTGCTGCTGCTGCTGCTCCTGGCCTGCCTGTTG
CCGGCGTCCGAGGATGACTACAGCTGCACCCAGGCCAACAACTTTGCCCG
ATCCTTCTACCCCATGCTGCGGTACACCAACGGGCCACCTCCCACCTAGC
AGATACCA
SEQ ID NO: 8:
CCCCTGGTT
SEQ ID NO: 9:
GGTTCATCACAA
SEQ ID NO: 10:
TTGCCTGCTTCAGAG
SEQ ID NO: 11:
CTAACGGTT
SEQ ID NO: 12:
GTGGATTCT
SEQ ID NO: 13:
GGTAGCAGCCAA
SEQ ID NO: 14:
TTGCCTGCCAGCGAG
SEQ ID NO: 15:
CTTTCTCTC
SEQ ID NO: 16:
GGTGGTACT
SEQ ID NO: 17:
GGCAGTAGTCAA
SEQ ID NO: 18:
TTGCCCGCTAGTGAG
SEQ ID NO: 19:
TCCCATCAT
SEQ ID NO: 20:
GGTTCCTTC
SEQ ID NO: 21:
GGCTCGTCGCAA
SEQ ID NO: 22:
TTGCCCGCCTCGGAG
SEQ ID NO: 23:
AAGTTGGCG
SEQ ID NO: 24:
GGTGGTACT
SEQ ID NO: 25:
GGATCTTCTCAA
SEQ ID NO: 26:
TTGCCAGCATCTGAG
SEQ ID NO: 27:
TCCCATCAT
SEQ ID NO: 28:
GGAGGCAAG
SEQ ID NO: 29:
GGGTCCTCCCAA
SEQ ID NO: 30:
TTGCCGGCGTCCGAG
SEQ ID NO: 31:
CATCAATCG
SEQ ID NO: 32:
TCGCAATCT
SEQ ID NO: 33:
GGTTCGTCGCAG
SEQ ID NO: 34:
CTCCCTGCATCGGAA
SEQ ID NO: 35:
ACGGCTACA
SEQ ID NO: 36:
CGCTACCAG
SEQ ID NO: 37:
GGTTCTTCTCAG
SEQ ID NO: 38:
CTCCCTGCTTCTGAA
SEQ ID NO: 39:
GCGTCGTAA
SEQ ID NO: 40:
ACAACACCT
SEQ ID NO: 41:
GGCTCCTCCCAG
SEQ ID NO: 42:
CTCCCCGCATCCGAA
SEQ ID NO: 43:
ATGACGACC
SEQ ID NO: 44:
AAAGTCCCG
SEQ ID NO: 45:
GGCTCATCACAG
SEQ ID NO: 46:
CTCCCCGCGTCAGAA
SEQ ID NO: 47:
TCTCATCCG
SEQ ID NO: 48:
GACTTCTCT
SEQ ID NO: 49:
GGAAGCAGCCAG
SEQ ID NO: 50:
CTCCCAGCCAGCGAA
SEQ ID NO: 51:
TCCACGGTT
SEQ ID NO: 52:
ACTCCAACT
SEQ ID NO: 53:
GGGAGTAGTCAG
SEQ ID NO: 54:
CTCCCGGCCAGTGAA
SEQ ID NO: 55:
TTCCAGCTC
SEQ ID NO: 56:
CAGGCTGAA
SEQ ID NO: 57:
GGTAGTTCTCAG
SEQ ID NO: 58:
TTGCCTGCATCTGAA
SEQ ID NO: 59:
TTCGCATTG
SEQ ID NO: 60:
CGTCGATGC
SEQ ID NO: 61:
GGCAGCTCCCAA
SEQ ID NO: 62:
TTGCCAGCTAGCGAG
SEQ ID NO: 63:
GACTCCACT
SEQ ID NO: 64:
GTTCGGAAA
SEQ ID NO: 65:
GGGAGCTCCCAG
SEQ ID NO: 66:
TTGCCGGCAAGTGAG
SEQ ID NO: 67:
ACTCCGTCG
SEQ ID NO: 68
AATGATACGGCGACCACCGAGATCTACACTAGATCGCACACTCTTTCCCT
ACACGACGCTCTTCCGATCT
SEQ ID NO: 69
GTGACTGGAGTTCAGACGTGTGCTCTTCCGATCTGATCCTTCTACCCCAT
GCTGCGG
SEQ ID NO: 70
CAAGCAGAAGACGGCATACGAGATxxxxxxxxGTGACTGGAGTTCAGACG
TGTGCTCTTCCGATC

### EXAMPLES

These examples are provided for illustrative purposes only and not to limit the scope of the claims provided herein.

### Example 1

### Multiplexing Regulatory Elements (REs) in an In Vivo AAV-Based Infection to Evaluate Specificity of REs

Multiple regulatory elements were assayed in an in vivo AAV-based system in order to evaluate the cell specificity of the individual regulatory elements. This assay allows for the identification of cell specific regulatory elements and the magnitude of expression of each transgene under the cell specific regulatory element.

### Design, Production and In Vivo Testing of Multiplexed RE AAVs

To test the ability of the system to multiplex three regulatory elements, a transgene of interest was operably linked to one of three following candidate REs: (1) CamKII, (2) CBA and (3) a regulatory element encoded by the nucleic acid sequence of SEQ ID NO: 1 (RE1). These REs were chosen with the understanding that the CamKII promoter exhibits preferential expression in excitatory neurons, the CBA promoter exhibits ubiquitous expression, and the regulatory element encoded by the nucleic acid sequence of SEQ ID NO: 1 (RE1) exhibits preferential expression in inhibitory/paravalbumin (PV) neurons. The transgene consisted of the reporter gene encoding an EGFP protein fused to a KASH nuclear tethering domain (EGFP-KASH). Three specific regions of KASH in the EGFP-KASH transgene were sequence modified to allow for individual identification in a mixed pool (Table 1). These sequence modifications only affected the DNA and RNA sequence of EGFP-KASH and did not vary the amino acid sequence. Therefore, the sequence modifications serve as a unique barcode for a given RE driving the respective EGFP-KASH transgene construct. The barcoded transgenes were cloned into an AAV genome backbone, and plasmids were assessed by transiently transfected HEK293 cells and EGFP fluorescence was evaluated. The barcoding strategy is shown in Table 1 below in which the barcoded regions of the KASH sequence are denoted in bold and underline.

TABLE 1

| Barcode | Sequence |
|---|---|
| MBC | GAGGAGGAGGAGGAGACAGACAGCAGGATGCCCCACC |
| 1 | TCGACAGCCCCGGTTCATCACAACCGAGACGCTCCTT |
| | CCTCTCAAGGGTGATCAGGGCAGCTCTACCGTTGCAG |
| | CTGCTTCTGCTGCTGCTGCTGCTCCTGGCCTGCCTGT |
| | TGCCTGCTTCAGAGGATGACTACAGCTGCACCCAGGC |
| | CAACAACTTTGCCCGATCCTTCTACCCCATGCTGCGG |
| | TACACCAACGGGCCACCTCCCACCTAGGACTCAGCT |
| | (SEQ ID NO: 2) |
| MBC | GAGGAGGAGGAGGAGACAGACAGCAGGATGCCCCACC |
| 2 | TCGACAGCCCCGGTAGCAGCCAACCGAGACGCTCCTT |
| | CCTCTCAAGGGTGATCAGGGCAGCTCTACCGTTGCAG |
| | CTGCTTCTGCTGCTGCTGCTGCTCCTGGCCTGCCTGT |
| | TGCCTGCCAGCGAGGATGACTACAGCTGCACCCAGGC |
| | CAACAACTTTGCCCGATCCTTCTACCCCATGCTGCGG |
| | TACACCAACGGGCCACCTCCCACCTAGCTTACTAGC |
| | (SEQ ID NO: 3) |
| MBC | GAGGAGGAGGAGGAGACAGACAGCAGGATGCCCCACC |
| 3 | TCGACAGCCCCGGCAGTAGTCAACCGAGACGCTCCTT |
| | CCTCTCAAGGGTGATCAGGGCAGCTCTACCGTTGCAG |
| | CTGCTTCTGCTGCTGCTGCTGCTCCTGGCCTGCCTGT |
| | TGCCCGCTAGTGAGGATGACTACAGCTGCACCCAGGC |
| | CAACAACTTTGCCCGATCCTTCTACCCCATGCTGCGG |
| | TACACCAACGGGCCACCTCCCACCTAGTCAGGAATC |
| | (SEQ ID NO: 4) |
| MBC | GAGGAGGAGGAGGAGACAGACAGCAGGATGCCCCACC |
| 4 | TCGACAGCCCCGGCTCGTCGCAACCGAGACGCTCCTT |
| | CCTCTCAAGGGTGATCAGGGCAGCTCTACCGTTGCAG |
| | CTGCTTCTGCTGCTGCTGCTGCTCCTGGCCTGCCTGT |
| | TGCCCGCCTCGGAGGATGACTACAGCTGCACCCAGGC |
| | CAACAACTTTGCCCGATCCTTCTACCCCATGCTGCGG |
| | TACACCAACGGGCCACCTCCCACCTAGAGACAGGTA |
| | (SEQ ID NO: 5) |
| MBC | GAGGAGGAGGAGGAGACAGACAGCAGGATGCCCCACC |
| 5 | TCGACAGCCCCGGATCTTCTCAACCGAGACGCTCCTT |
| | CCTCTCAAGGGTGATCAGGGCAGCTCTACCGTTGCAG |
| | CTGCTTCTGCTGCTGCTGCTGCTCCTGGCCTGCCTGT |
| | TGCCAGCATCTGAGGATGACTACAGCTGCACCCAGGC |
| | CAACAACTTTGCCCGATCCTTCTACCCCATGCTGCGG |
| | TACACCAACGGGCCACCTCCCACCTAGGATTCTCAG |
| | (SEQ ID NO: 6) |
| MBC | GAGGAGGAGGAGGAGACAGACAGCAGGATGCCCCACC |
| 6 | TCGACAGCCCCGGGTCCTCCCAACCGAGACGCTCCTT |
| | CCTCTCAAGGGTGATCAGGGCAGCTCTACCGTTGCAG |
| | CTGCTTCTGCTGCTGCTGCTGCTCCTGGCCTGCCTGT |
| | TGCCGGCGTCCGAGGATGACTACAGCTGCACCCAGGC |
| | CAACAACTTTGCCCGATCCTTCTACCCCATGCTGCGG |
| | TACACCAACGGGCCACCTCCCACCTAGCAGATACCA |
| | (SEQ ID NO: 7) |

To set up the initial multiplexing experiment (as illustrated in the simplified schematic of FIG. 1), two barcodes for each RE were assigned, and a plasmid mix was made comprising equal quantities of each barcoded construct (e.g., CamKII-EGFP-KASH barcode 1, CamKII-EGFP-KASH barcode 2, CBA-EGFP-KASH barcode 3, CBA-EGFP-KASH barcode 4, RE1-EGFP-KASH barcode 5, and RE1-EGFP-KASH barcode 6). This mix (referred to as L1) was used to produce adeno-associated virus 9 (AAV9), the selected in vivo delivery vehicle. Wildtype (C571B16/5) mice (n=6) were infused bilaterally with the AAV vectors into the dorsal and ventral hippocampus (4 injections sites) with AAV9 L1 (2E14 genome copies (gc)/mouse), or a PBS control. Four weeks post injection, animals were sacrificed and right and left hippocampi were surgically removed and stored in RNAlater™ at 4° C. overnight.

Individual mouse hippocampi were homogenized in lysis buffer via manual douncing, in order to release nuclei. Concentrated, crude nuclei preparations were obtained by PBS-based washes and centrifugation. Nuclei were stained with DAPI for identification on the cell sorter and to confirm nuclei integrity. Nuclei were purified using a BD FACSAria™ II cell sorter, with the PBS-injected control sample used to define the gating strategy. For every sample, approximately 100,000 nuclei were sorted, and samples were concentrated by centrifugation for single nucleus RNA sequencing (RNAseq). Single nucleus RNAseq was performed with the 10× genomics Chromium Single Cell 3′ v2 kit. The resulting cDNA libraries underwent next generation sequencing.

### Sequence Processing

Post sequencing, raw BCL sequence files (Illumina binary format) were downloaded from Illumina BaseSpace and converted into raw FASTQ read files using customized processing scripts. For each sample, raw FASTQs along with mouse genome and gene annotations (GENCODE version M19, https://uswest.ensembl.org/Mus_musculus/Info/Annotation) were processed using 10× Cell Ranger software (v. 2.1.0). 10× Cell Ranger software demultiplexes the reads by cell and then maps reads to transcripts. For mapping reads to transcripts, a pre-mRNA reference transcriptome was used because a large fraction of transcripts from the nuclear samples are pre-mRNAs. For reads deriving from the AAV vectors, each barcoded AAV transcript sequence was manually added to the reference transcriptome. 10× Cell Ranger generated a file for each sample containing unique molecular identifier (UMI) counts for each gene in each detected nucleus. These UMI count files were then used for dimensionality reduction and clustering to define tissue sub-populations.

### Sequencing Analysis

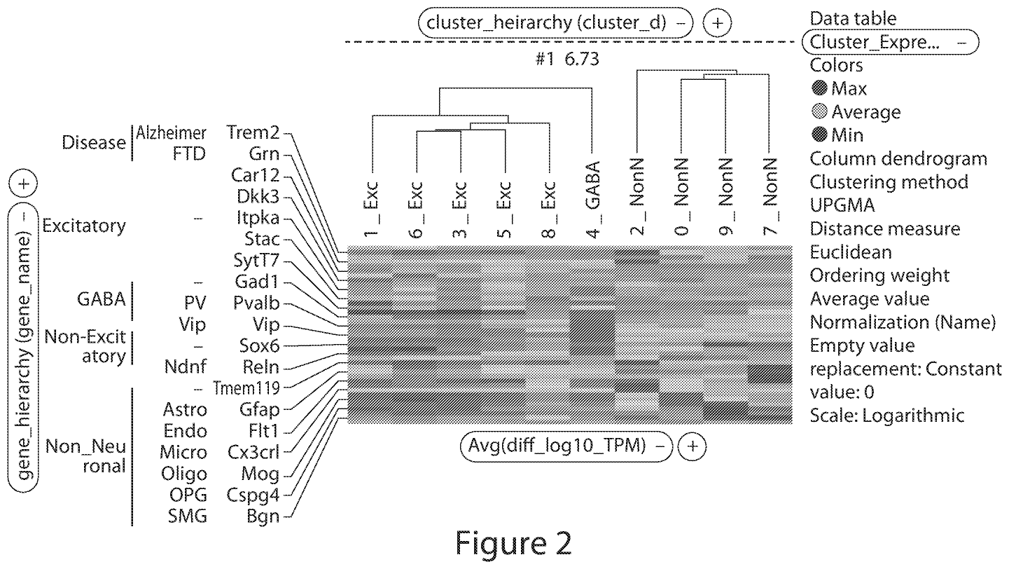

The UMI count files from above were processed using custom R and Python scripts to identify cellular sub-populations. The cell-by-gene count files were first filtered to remove cells that contain less than 300 UMIs in total. The filtered 2D matrix of UMI counts by cell (rows) and genes (columns) was reduced to a smaller size with the same number of cells (rows) but the gene columns replaced by 35 reduced dimensions using ZinbWave (version 1.3.4, D. Risso et al., Nature 9: 284 (2018)). The 35 reduced dimensions are linear combinations of genes and represent biological modules that are active in different cell types. By reducing the dimensionality from −15K genes to 35 biological modules, noise in the data was significantly reduced, effectively alleviating the well-known ‘drop-out’ issue of single cell data, thereby making the clustering more tractable. The top 5000 variable genes (as calculated by Seurat: https:satijalab.org/seurat/, with parameters min.cells=300, min.genes=200, y.cutoff=0.005) were used to calculate the 35 dimensions using ZinbWave (default parameters). In addition, the total transcriptional output of each cell (total UMI) was incorporated as a covariate in the ZinbWave method. 11971 For clustering this matrix, the Louvain clustering algorithm as implemented in the package Louvain (version 0.6.1, https://pypi.org/project/louvain/) was used. Louvain algorithm requires as input a graph, with cells as vertices connected by edges. The graphs were constructed by including an edge between two cells if their correlation (using the 35 dimensional representation) was greater than 0.5. The identified clusters (or cellular sub-populations) were then annotated based on literature-derived canonical biomarkers (see Table 2 and FIG. 2). Comparative analysis of EGFP-KASH expression in neuronal populations was performed to evaluate the effect that a given RE has on transgene expression.

TABLE 2

| | Gene Sub- | | Gene | Chromosome | Chromosome |
|---|---|---|---|---|---|
| Gene Label | label | Gene ID | Name | start | end |
| Disease | FTD | ENSMUSG00000034708 | Grn | 102430314 | 102437048 |
| Disease | Dravet | ENSMUSG00000064329 | Scn1a | 66270777 | 66440840 |
| Disease | Alzheimer | ENSMUSG00000023992 | Trem2 | 48346400 | 48354147 |
| Excitatory | — | ENSMUSG00000032502 | Stac | 111561436 | 111690348 |
| Excitatory | — | ENSMUSG00000070570 | Slc17a7 | 45163948 | 45176142 |
| Excitatory | — | ENSMUSG00000032373 | Car12 | 66713685 | 66766845 |
| Excitatory | — | ENSMUSG00000058420 | Syt17 | 118380716 | 118448222 |
| Excitatory | — | ENSMUSG00000027296 | Itpka | 119742336 | 119751263 |
| Excitatory | — | ENSMUSG00000001119 | Col6a1 | 76708791 | 76726168 |
| Excitatory | — | ENSMUSG00000024617 | Camk2a | 60925617 | 60988152 |
| Excitatory | — | ENSMUSG00000053025 | Sv2b | 75114893 | 75309262 |
| Excitatory | — | ENSMUSG00000041324 | Inhba | 16011850 | 16027211 |
| Excitatory | — | ENSMUSG00000030772 | Dkk3 | 112116016 | 112159057 |
| GABA | — | ENSMUSG00000070880 | Gad1 | 70553071 | 70602014 |
| GABA | Vip | ENSMUSG00000019772 | Vip | 5639217 | 5647617 |
| GABA | — | ENSMUSG00000062209 | Erbb4 | 68032185 | 69108059 |
| GABA | Sst | ENSMUSG00000004366 | Sst | 23889580 | 23890844 |
| GABA | — | ENSMUSG00000026787 | Gad2 | 22622204 | 22693874 |
| GABA | PV | ENSMUSG00000005716 | Pvalb | 78191113 | 78206400 |
| Non_Excitatory | Ndnf | ENSMUSG00000042453 | Reln | 21884453 | 22344702 |
| Non_Excitatory | — | ENSMUSG00000051910 | Sox6 | 115470871 | 116038796 |
| Non_Excitatory | Ndnf | ENSMUSG00000049001 | Ndnf | 65671589 | 65712326 |
| Non_Excitatory | — | ENSMUSG00000037771 | Slc32a1 | 158610766 | 158615748 |
| Non_Neuronal | Astro | ENSMUSG00000020932 | Gfap | 102887335 | 102900912 |
| Non_Neuronal | Endo | ENSMUSG00000029648 | Flt1 | 147561603 | 147726011 |
| Non_Neuronal | OPC | ENSMUSG00000029231 | Pdgfra | 75152291 | 75198215 |
| Non_Neuronal | Oligo | ENSMUSG00000046160 | Olig1 | 91269771 | 91271933 |
| Non_Neuronal | OPC | ENSMUSG00000032911 | Cspg4 | 56865032 | 56899870 |
| Non_Neuronal | — | ENSMUSG00000033208 | S100b | 76253852 | 76261159 |
| Non_Neuronal | — | ENSMUSG00000054675 | Tmem119 | 113793728 | 113800516 |
| Non_Neuronal | Micro | ENSMUSG00000038642 | Ctss | 95526785 | 95556403 |
| Non_Neuronal | Micro | ENSMUSG00000052336 | Cx3cr1 | 119901615 | 120069879 |
| Non_Neuronal | Oligo | ENSMUSG00000076439 | Mog | 37010742 | 37023398 |
| Non_Neuronal | SMC | ENSMUSG00000031375 | Bgn | 73483601 | 73495933 |
| Non_Neuronal | Oligo | ENSMUSG00000036634 | Mag | 30899175 | 30914873 |
| Non_Neuronal | Astro | ENSMUSG00000050953 | Gja1 | 56377299 | 56390419 |
| Non_Neuronal | Astro | ENSMUSG00000024411 | Aqp4 | 15389393 | 15403684 |
| Pan_Neuronal | — | ENSMUSG00000027273 | Snap25 | 136713452 | 136782428 |

### Results

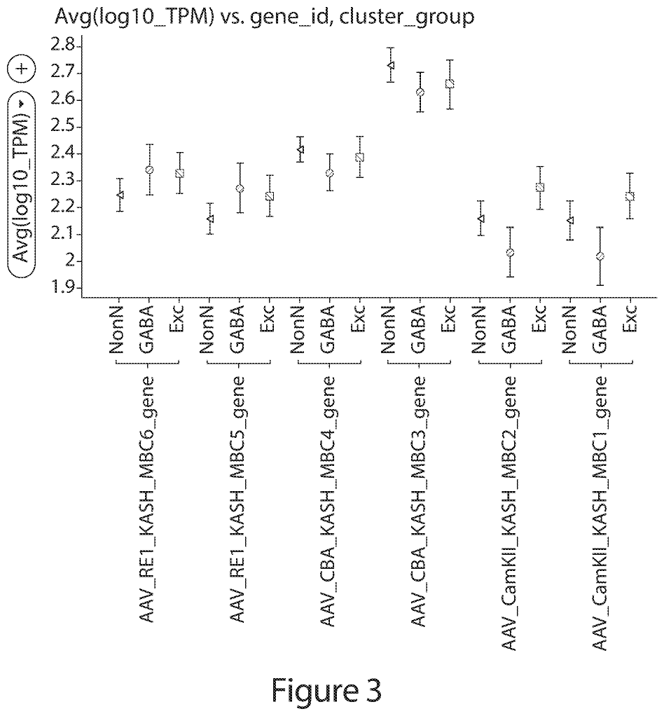

Based on known biomarkers for each sample, the clusters were grouped into three cluster-groups—Excitatory neurons (Exc), GABAergic neurons (GABA), and Non-Neuronal cells (NonN). For ease of interpretation, each of these cluster-groups are referred to as cell populations. From UMI counts, the expression of each barcoded AAV transgene in Transcripts-Per-Million (TPM) was calculated (FIG. 3).

The Gene TPM was calculated as follows:

$$Gene TPM = \frac{{10}^{6} \times ( gene UMI counts within a cell population )}{( \begin{matrix}total UMI counts across all genes \\ within a cell population\end{matrix} )}$$

To be able to compare expression within GABA versus Excitatory and to more easily compare different RE-driven AAV transgenes, the TPM expression of all AAV genes were normalized to their expression in excitatory neurons. Since CBA was utilized as a ubiquitously-expressed positive control, the TPM expression of each AAV gene was also normalized within a cell population to the average TPM expression of the AAV CBA transgene within that population. Finally, for ease of interpretation, the relative expression of each AAV transgene (normalized to CBA) was expressed as a CBA-Normalized Fold Change.

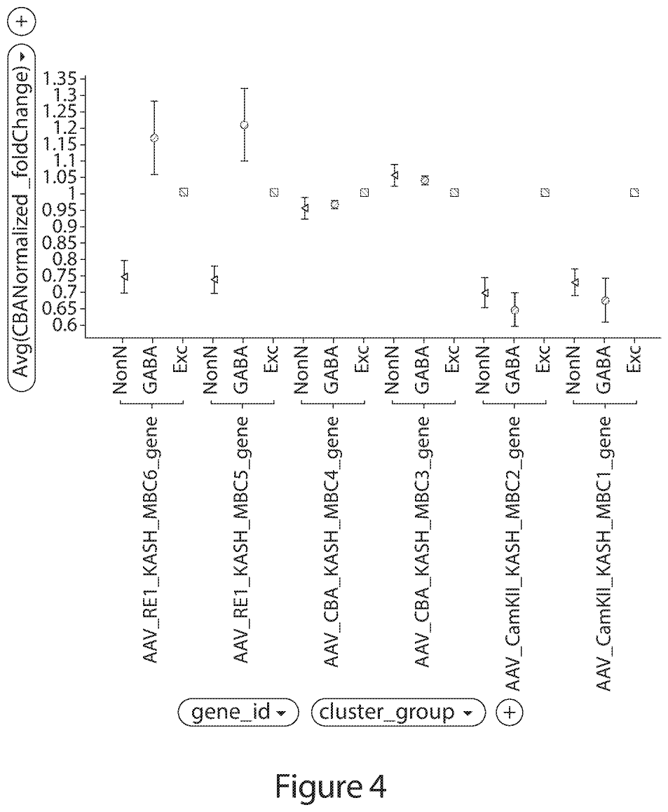

As expected, the relative expression of the two CamKII AAV transgenes is ˜30% lower in GABA and non-neuronal populations as compared to excitatory cells (FIG. 4). The two RE1 driven AAV transgenes are ˜20% higher in GABA neurons compared to excitatory neurons, and ˜25% lower in non-neuronal cells.

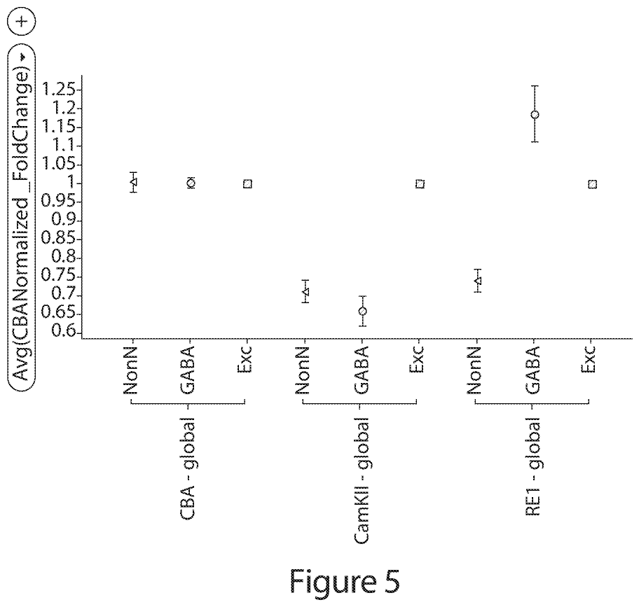

Additionally, since the two barcoded constructs for each AAV transgene show similar expression within each cell population, the expression values between the two barcoded constructs for each AAV transgene were averaged to obtain a simplified expression plot (FIG. 5).

Similar to FIG. 4, FIG. 5 demonstrates that the relative expression of CamKII AAV transgene is ˜30% lower in GABA and non-neuronal populations as compared to excitatory cells, and that the RE1 driven AAV transgene is ˜20% higher in GABA neurons compared to excitatory neurons, and ˜25% lower in non-neuronal cells.

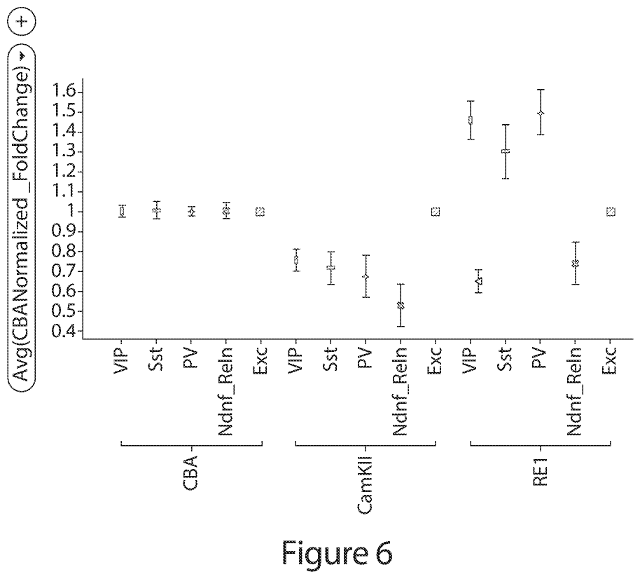

The four major sub-populations of GABAergic neurons were evaluated using known biomarkers (PV, VIP, Sst, Ndnf-Reln). The results demonstrate that the expression of the 106 ml transgene is considerably higher within PV, VIP and Sst sub-populations of GABA (FIG. 6). Additionally, the results demonstrated that the average fold-change in the PV sub-population is highest for RE1 (˜50% higher than in excitatory cells).

These data obtained using the method described above demonstrate that candidate regulatory elements can be screened in vivo to identify cell specific regulatory elements and the magnitude of expression of each transgene under the cell specific regulatory element. Furthermore, the results show that these methods can effectively be used for performing multiplexed analysis of regulatory elements in order to identify regulatory elements which achieve a physiologically relevant dose in a specific population of cells. The assay described herein could be useful in screening upwards of 10⁴ candidate regulatory elements in an in vivo system using a variety of delivery methods.

### Example 2

### Use of REs Identified Using an In Vivo AAV-Based Infection to Evaluate Specificity of REs

After validating the cell selectivity of the regulatory elements identified using a screening assay described herein, the regulatory elements can be utilized for targeting a specific transgene to a specific population of cells. Specifically, each regulatory element can be operably linked to a transgene to target expression selectively to a specific cell population over at least one, two, three, four, five, or more than five non-PV cells.

### Example 3

### Multiplexing Regulatory Elements (REs) In Vivo at Large Scale to Evaluate Specificity of REs in Complex Mixtures

TABLE 3

| Regulatory Element | L3 Barcode | L3.2 Barcode |
|---|---|---|
| Construct 1 (CBA-EGFP-KASH) | MBC7 | MBC7 |
| Construct 2 (EF1α-EGFP-KASH) | MBC10 | MBC10 |
| Construct 3 (RE1-EGFP-KASH) | MBC11 | MBC11 |
| Construct 4 (RE2-EGFP-KASH) | MBC8 | MBC8 |
| Construct 5 (RE3-EGFP-KASH) | MBC9 | MBC9 |
| Construct 6 (RE4-EGFP-KASH) | MBC12 | MBC12 |
| Construct 7 (RES-EGFP-KASH) | MBC13 | MBC13 |
| Construct 8 (RE6-EGFP-KASH) | MBC14 | MBC14 |
| Construct 9 (RE7-EGFP-KASH) | MBC15 | MBC15 |
| Construct 10 (RE8-EGFP-KASH) | MBC16 | MBC16 |
| Construct 11 (RE9-EGFP-KASH) | MBC17 | MBC17 |
| Construct 12 (RE10-EGFP-KASH) | MBC18 | MBC18 |
| Construct 13 (RE11-EGFP-KASH) | MBC19 | MBC19 |
| Construct 14 (RE12-EGFP-KASH) | MBC20 | N/A |
| Construct 15 (RE13-EGFP-KASH) | MBC21 | MBC21 |

To test whether the multiplex assay was capable of evaluating cell type specificity and magnitude of expression of individual REs in complex mixtures of cells, fifteen regulatory elements were assayed in an in vivo AAV-based system. This assay allows for the identification of cell specific regulatory elements as well as the magnitude of expression of each transgene under the cell specific regulatory element, within a complex mixture of multiple different constructs.

### Design, Production and In Vivo Testing of Multiplexed RE AAVs

To test the ability of the system to multiplex a complex mixture of regulatory elements, a transgene of interest was operably linked to one of fifteen candidate REs. Two of the REs were CBA and EF1α, which were both selected as ubiquitously expressed control promoters (Construct 1 and Construct 2, respectively). The regulatory element encoded by the nucleic acid sequence of SEQ ID NO: 1 (RE1), which exhibits preferential expression in inhibitory/paravalbumin (PV) neurons, was used in Construct 3. See Table 3. The remaining twelve promoters were selected for their preferential expression in inhibitory/PV neurons. The transgene consisted of the reporter gene encoding an EGFP protein fused to a KASH nuclear tethering domain (EGFP-KASH). Two regions of the coding sequence of KASH (KASH Sequence 1 and KASH Sequence 2) in the EGFP-KASH transgene were sequence modified to allow for individual identification in a mixed pool (Table 4). These sequence modifications only affected the DNA and RNA sequence of EGFP-KASH and did not vary the amino acid sequence. Therefore, the sequence modifications serve as a unique barcode for a given RE driving the respective EGFP-KASH transgene construct. An additional unique barcode sequence was inserted upstream of the transcription start site for each construct to allow for individual identification of a specific construct in a mixed pool (Table 4, Upstream Sequence). Finally, a unique barcode sequence was inserted after the stop codon of the EGFP transgene for each construct to allow for individual identification of a specific construct in a mixed pool (Table 4, Downstream Sequence). The barcoded transgenes were cloned into an AAV genome backbone and used to prepare AAV9 virus for in vivo studies. The unique barcode sequences are shown in Table 4 below.

TABLE 4

| | Upstresam | KASH | KASH | Downstream |
|---|---|---|---|---|
| Barcode | Sequence | Sequence 1 | Sequence 2 | Sequence |
| MBC7 | CCCCTGGTT | GGTTCATCACAA | TTGCCTGCTTCAGAG | CTAACGGTT |
| | (SEQ ID NO: 8) | (SEQ ID NO: 9) | (SEQ ID NO: 10) | (SEQ ID NO: 11) |
| MBC8 | GTGGATTCT | GGTAGCAGCCAA | TTGCCTGCCAGCGAG | CTTTCTCTC |
| | (SEQ ID NO: 12) | (SEQ ID NO: 13) | (SEQ ID NO: 14) | (SEQ ID NO: 15) |
| MBC9 | GGTGGTACT | GGCAGTAGTCAA | TTGCCCGCTAGTGAG | TCCCATCAT |
| | (SEQ ID NO: 16) | (SEQ ID NO: 17) | (SEQ ID NO: 18) | (SEQ ID NO: 19) |
| MBC10 | GGTTCCTTC | GGCTCGTCGCAA | TTGCCCGCCTCGGAG | AAGTTGGCG |
| | (SEQ ID NO: 20) | (SEQ ID NO: 21) | (SEQ ID NO: 22) | (SEQ ID NO: 23) |
| MBC11 | GGTGGTACT | GGATCTTCTCAA | TTGCCAGCATCTGAG | TCCCATCAT |
| | (SEQ ID NO: 24) | (SEQ ID NO: 25) | (SEQ ID NO: 26) | (SEQ ID NO: 27) |
| MBC12 | GGAGGCAAG | GGGTCCTCCCAA | TTGCCGGCGTCCGAG | CATCAATCG |
| | (SEQ ID NO: 28) | (SEQ ID NO: 29) | (SEQ ID NO: 30) | (SEQ ID NO: 31) |
| MBC13 | TCGCAATCT | GGTTCGTCGCAG | CTCCCTGCATCGGAA | ACGGCTACA |
| | (SEQ ID NO: 32) | (SEQ ID NO: 33) | (SEQ ID NO: 34) | (SEQ ID NO: 35) |
| MBC14 | CGCTACCAG | GGTTCTTCTCAG | CTCCCTGCTTCTGAA | GCGTCGTAA |
| | (SEQ ID NO: 36) | (SEQ ID NO: 37) | (SEQ ID NO: 38) | (SEQ ID NO: 39) |
| MBC15 | ACAACACCT | GGCTCCTCCCAG | CTCCCCGCATCCGAA | ATGACGACC |
| | (SEQ ID NO: 40) | (SEQ ID NO: 41) | (SEQ ID NO: 42) | (SEQ ID NO: 43) |
| MBC16 | AAAGTCCCG | GGCTCATCACAG | CTCCCCGCGTCAGAA | TCTCATCCG |
| | (SEQ ID NO: 44) | (SEQ ID NO: 45) | (SEQ ID NO: 46) | (SEQ ID NO: 47) |
| MBC17 | GACTTCTCT | GGAAGCAGCCAG | CTCCCAGCCAGCGAA | TCCACGGTT |
| | (SEQ ID NO: 48) | (SEQ ID NO: 49) | (SEQ ID NO: 50) | (SEQ ID NO: 51) |
| MBC18 | ACTCCAACT | GGGAGTAGTCAG | CTCCCGGCCAGTGAA | TTCCAGCTC |
| | (SEQ ID NO: 52) | (SEQ ID NO: 53) | (SEQ ID NO: 54) | (SEQ ID NO: 55) |
| MBC19 | CAGGCTGAA | GGTAGTTCTCAG | TTGCCTGCATCTGAA | TTCGCATTG |
| | (SEQ ID NO: 56) | (SEQ ID NO: 57) | (SEQ ID NO: 58) | (SEQ ID NO: 59) |
| MBC20 | CGTCGATGC | GGCAGCTCCCAA | TTGCCAGCTAGCGAG | GACTCCACT |
| | (SEQ ID NO: 60) | (SEQ ID NO: 61) | (SEQ ID NO: 62) | (SEQ ID NO: 63) |
| MBC21 | GTTCGGAAA | GGGAGCTCCCAG | TTGCCGGCAAGTGAG | ACTCCGTCG |
| | (SEQ ID NO: 64) | (SEQ ID NO: 65) | (SEQ ID NO: 66) | (SEQ ID NO: 67) |

The multiplex of complex mixtures was set up similar to the initial experiment described in Example 1, except a single MBC barcode comprising a unique upstream sequence, two unique sequences internal to KASH, and a unique downstream sequence was assigned for each RE, and a plasmid mix was made comprising equal quantities of each barcoded construct (e.g., MBC7-CBA-EGFP-KASH, MBC10-EF1α-EGFP-KASH, MBC11-RE1-EGFP1-KASH, etc.). This mix (referred to as L3) was used to produce adeno-associated virus 9 (AAV9), the selected in vivo delivery vehicle. The experiment was repeated a second time using the same unique bar code sequences, except that the sequence segments that comprised a barcode (e.g., upstream sequence, two sequences internal to KASH, and downstream sequence) were configured within the constructs differently. A plasmid mix was made comprising equal quantities of each of these barcoded constructs. This mix (referred to as L3.2) was used to produce additional AAV9. The L3.2 library did not include Construct 14.

Six to eight week old wildtype (C57B16/J) male mice were infused unilaterally with 1.5 μL of AAV vector pools into the dorsal and ventral cortex, and 1.5 μL into the dorsal and ventral hippocampus (2 injection sites with 3 μL per site; 1.5 μL for hippocampus and 1.5 μL for cortex) with AAV9 L3 or AAV9 L3.2 at a genome content of 1.5×10¹¹ to 2.4×10¹¹ viral genomes/mouse (vg/mouse) at a rate of 0.3 μL/minute with a 4 minute rest period following injection.

Four weeks post injection, animals were sacrificed, and their sensory cortex and hippocampus were surgically removed, stored in RNAlater™ at 4° C. for 24 hours, and then frozen at −80° C. until the tissue was ready to process.

RNAlater™ brain cortex or hippocampus samples were thawed on ice. In order to release nuclei, approximately 20 milligrams of tissue was manually homogenized in lysis buffer. Concentrated, crude nuclei preparations were obtained by PBS-based washes and centrifugation. Nuclei were stained with DAPI for identification on the cell sorter and to confirm nuclei integrity. Nuclei were purified using a BD FACS Melody cell sorter. For every sample, approximately 100,000 nuclei were sorted. Samples were concentrated by centrifugation for single nucleus RNAseq. Single nucleus RNAseq was performed with the 10× Genomics Chromium Single Cell 3′ v3 kit (as described in the manufacturer's instructions—FIG. 1). The resulting cDNA libraries underwent next generation sequencing.

In order to increase detection of AAV constructs with UMIs that fall below the threshold of detection in single nucleus RNAseq, an enrichment PCR step was performed on the cDNA samples from the 10× workflow prior to amplification. This enrichment step produced a 3-10-fold amplification of the signal from AAV constructs that was detected from the 10× libraries. The PCR primers used in the enrichment PCR step included a forward primer from the standard Illumina Truseq sequencing primer (501) and a reverse primer that was designed to bind to a region in the AAV transgene relatively close to the polyA site. This reverse primer had a Read 2 handle added to it, so that it could be used in a subsequent PCR reaction as a means to add an Illumina adaptor to the product (for sequencing purposes). This step is referred to herein as pullout PCR. The primer sequences for this pullout PCR are shown in Table 5.

TABLE 5

| Primer Name | Primer sequence | Primer usage |
|---|---|---|
| 501 Illumina | AATGATACGGCGACCACCGAGATCTA | Illumina sequencing adaptor |
| primer | CACTAGATCGCACACTCTTTCCCTAC | including p5 and Read 1 |
| | ACGACGCTCTTCCGATCT | sequences |
| | (SEQ ID NO: 68) | |
| Perturb_KASH_ | GTGACTGGAGTTCAGACGTGTGCTCT | Reverse primer specific to a |
| 2F | TCCGATCTGATCCTTCTACCCCATGC | region in KASH with Read 2 |
| | TGCGG (SEQ ID NO: 69) | handle |
| 70x Illumina | CAAGCAGAAGACGGCATACGAGATxx | Illumina sequencing adaptor |
| primer | xxxxxxGTGACTGGAGTTCAGACGTG | including p7 and Read 2 |
| | TGCTCTTCCGATC | sequences |
| | (SEQ ID NO: 70) | |

The 10× Genomics Chromium Single Cell 3′ v3 kit workflow improves sensitivity and allows detection of DNA/protein information on a single cell level. The beads that are incorporated into the single nucleus droplets for cDNA production are modified in the v3 workflow. These beads are engineered to capture polyA sequences as well as DNA/RNA sequences that incorporate a Capture 1 or Capture 2 sequence. This facilitates detection of antibody-oligo conjugates for specific proteins of interest as well as DNA species incorporating these capture sequences. In order for the kit to capture these DNA/RNA species and uniquely link them to the RE in a given construct, a unique barcode feature is encoded next to the capture sequence. This barcode is unique to each RE.

In the 10× Genomics Chromium Single Cell 3′ v3 kit workflow, each sample contains four sample indexes for demultiplexing. In pullout PCR, each sample contains only one sample index. To process pullout PCR samples through 10× Cell Ranger software used in the 10× Genomics Chromium Single Cell 3′ v3 kit workflow, the one pullout PCR sample index was combined with three “shan” indexes (different by at least two nucleotides to any 10× index) to mimic the four-sample index requirement by 10× Cell Ranger software. After demultiplexing into 10×-compatible FASTQ files, processing proceeds exactly as 10× sequence processing.

### Sequence Processing

Post sequencing, raw BCL sequence files (Illumina binary format) were downloaded from Illumina BaseSpace and converted into raw FASTQ read files using 10× Cell Ranger software (v.3.0.2) to demultiplex samples, where each sample has four 10× indexes. For each sample, raw FASTQs along with mouse genome and gene annotations (GENCODE version M19, https://uswest.ensembl.org/Mus_musculus/Info/Annotation) were processed using 10× Cell Ranger software (v.3.0.2). 10× Cell Ranger software demultiplexes the reads by cell and then maps reads to transcripts. FASTQ files contain paired-end reads, with Read 1 containing the UMI barcode and 10× cell barcode and Read 2 containing the gene transcript sequence. Read 2 is aligned to the mouse genome and each RE sequence to determine gene/RE identity. The 10× Cell Ranger software generated a file for each sample containing unique molecular identifier (UMI) counts for each gene in each detected nucleus. These UMI count files were then used for dimensionality reduction and clustering in order to define tissue sub-populations.

### Sequencing Analysis

For dimensionality reduction, the top 5000 variable genes (as calculated according to Stuart, Butler et al., bioRxiv, 2018; Butler et al, Nature Biotechnology, 2018; Hafemeister and Satija, bioRxiv 2019; with parameters min.cells=300, min.genes=200, y.cutoff=0.005) were used to calculate 35 dimensions using ZinbWave (default parameters). In addition, the total transcriptional output of each cell (total UMI) was incorporated as a covariate in the ZinbWave method. Similar to processing of the L1 library, the UMI count files were processed using custom R and Python scripts to identify cellular sub-populations. The cell-by-gene count files were first filtered to remove cells that contain less than 300 UMIs in total. The filtered 2D matrix of UMI counts by cell (rows) and genes (columns) was reduced to a smaller size with the same number of cells (rows) but the gene columns replaced by 35 reduced dimensions using ZinbWave (version 1.3.4, D. Risso et al., Nature 9: 284 (2018)). The 35 reduced dimensions are linear combinations of genes and represent biological modules that are active in different cell types. By reducing the dimensionality from ˜15K genes to 35 biological modules, noise in the data was significantly reduced, effectively alleviating the well-known ‘drop-out’ issue of single cell data, thereby making the clustering more tractable.

For clustering this matrix, the Louvain clustering algorithm, as implemented in the package Louvain (version 0.6.1, https://pypi.org/project/louvain/), was used as described above. Louvain algorithm requires a graph as input, with cells as vertices connected by edges. The graphs were constructed by including an edge between two cells if their correlation (using the 35 dimensional representation) was greater than 0.5. The identified clusters (or cellular sub-populations) were then annotated based on literature-derived canonical biomarkers for GABAergic neurons, excitatory neurons, and non-neuronal cell populations as indicated in Table 2 and FIG. 2. Comparative analysis of EGFP-KASH expression in neuronal populations was performed to evaluate the relative magnitude of expression and cell type specificity that a given RE has on transgene expression.

### Results

As described in Example 1, the clusters were grouped into three cluster-groups based on known biomarkers for each sample: Excitatory neurons (Exc), GABAergic neurons (GABA), and Non-Neuronal cells (NonN). From UMI counts, the expression of each barcoded AAV transgene in Transcripts-Per-Million (TPM) was calculated using the Gene TPM algorithm discussed above.

Initially, TPM in both L3 and L3.2 libraries was analyzed from each RE in excitatory and GABAergic neurons to determine the magnitude of gene expression and cell type specificity from each RE in excitatory and GABAergic neurons. The magnitude of expression provides feedback on the strength of a RE. Cell type specificity for excitatory or GABAergic neurons is also displayed, where differences in expression between excitatory and GABAergic neurons for a specific promoter indicate specificity for the respective cell type. For example, Construct 6 and Construct 3 show higher expression in GABAergic neurons, and therefore indicate that this RE is GABAergic neuron specific. However, Construct 1 shows relatively similar expression in both GABAergic and excitatory neurons, indicating a lack of cell type specificity of the promoter.

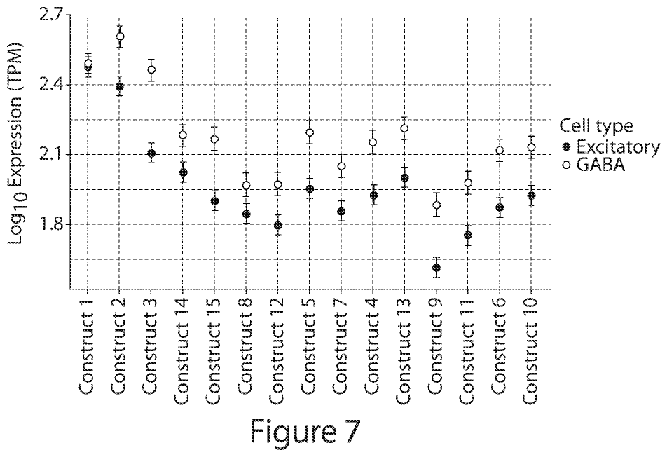

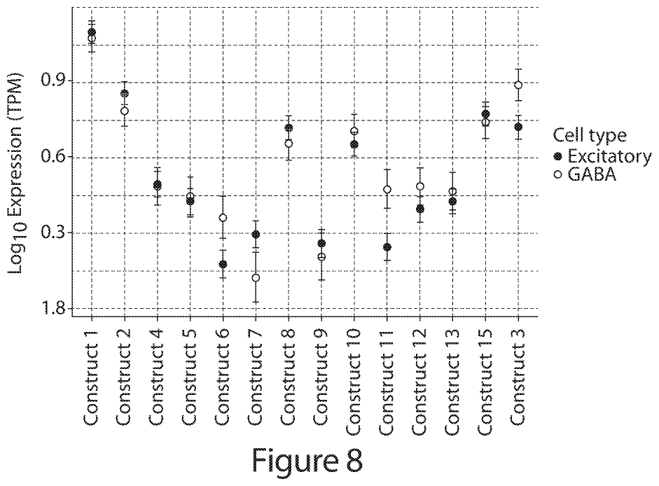

For ease of interpretation, the relative expression of each AAV transgene was presented in log scale. Increased expression from the CBA promoter (Construct 1) and EF1α promoter (Construct 2) was observed. This increased expression from the CBA and EF1α promoters was expected given that these promoters are known to be strong ubiquitous promoters. Increased expression was also observed from RE1 (Construct 3). Lower levels of expression were observed from the other candidate promoters, potentially indicating that these promoters drive gene expression less than CBA and EF1α. Interestingly, cell type specific expression in GABAergic neurons from the tested regulatory elements was observed for several of the constructs. See FIGS. 7 and 8. These data demonstrate that the multiplex assay is capable of detecting multiple REs in a single assay, as well as identify cell type-specific REs and their strength.

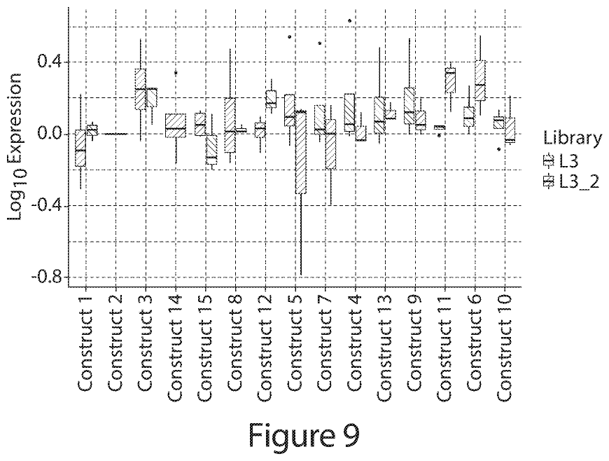

Cell type specific expression from each RE was next evaluated in both L3 and L3.2 libraries for specificity within GABAergic neurons (FIG. 9). Here, the TPM expression of each AAV gene was normalized within a cell population to the average TPM expression of the AAV EF1α associated transgene within that population since EF1α was utilized as a ubiquitously-expressed control. Furthermore, specificity for expression within GABAergic neurons was calculated relative to expression in excitatory neurons as follows:

log₁₀(specificity)=log₁₀(GABA neuron expression)−log₁₀(excitatory neuron expression)

For ease of interpretation, the relative expression of each AAV transgene (normalized to EF1α) was expressed as an EF1α-Normalized Fold Change and presented in log scale.

Since each TPM expression was normalized within a population to the average TPM expression of the EF1α associated transgene, the expression of EF1α is zero. Expression from the CBA promoter (Construct 1) is, on average, similar to expression from the EF1αpromoter. This is expected since CBA and EF1α are highly expressed ubiquitous promoters. In contrast, Construct 3 shows substantially higher expression in GABAergic neurons relative to excitatory neurons, as well as the ubiquitous expression from CBA and EF1α. This is also expected since Construct 3 utilizes an RE that exhibits preferential expression in inhibitory/paravalbumin (PV) neurons (RE1, encoded by SEQ ID NO: 1). The remaining constructs show higher expression in GABAergic neurons relative to excitatory neurons, as well as the ubiquitous expression from CBA and EF1α, although the expression is not as high as for Construct 3. This indicates that the REs in these constructs drive cell type specific expression in GABAergic neurons. These data demonstrate that the multiplex assay is capable of detecting multiple REs that drive GABAergic neuron specific expression.

The multiplex assay was tested for the ability to measure cell type-specific expression (AAV L3.2 library) within specific cell types within the class of GABAergic neurons (e.g., PV, SST, and VIP cells), instead of GABAergic neurons generally. TPM expression of each AAV gene was normalized within a cell population to the average TPM expression of the AAV EF1α associated transgene within that population since EF1α was utilized as a ubiquitously-expressed control. Specificity was also defined as described above. As expected, expression of the EF1α and CBA associated transgenes are similar and close to zero in all specific GABAergic cell types since they are ubiquitously expressed cells. The multiplex assay was also able to identify REs (e.g., Construct 11) that had higher transgene expression in all GABAergic cell types, indicating that these REs are not specific for certain cell types within class of GABAergic neurons (FIG. 10). Importantly, the multiplex assay was able to identify and delineate expression from certain REs that were specific for expression from certain cell types within the class of GABAergic neurons.

The data obtained using the method described above further demonstrate that candidate regulatory elements can be screened in complex mixtures of regulatory elements in vivo in order to identify cell-specific regulatory elements as well as the magnitude of expression of each transgene under the cell specific regulatory element. Furthermore, these results further show that the methods described herein can effectively be used for performing multiplexed analysis of regulatory elements in order to identify regulatory elements that achieve a physiologically relevant dose in a specific population of cells. The assay described herein could be useful in screening upwards of 10⁴ candidate regulatory elements in an in vivo system using a variety of delivery methods.

What is claimed is:

1. A method of identifying a regulatory element that provides selective expression in a given cell type, comprising:
a. providing cells with a mixture of vectors each comprising a candidate regulatory element operably linked to a transgene, wherein each vector further comprises a barcode;
b. isolating RNA from a plurality of single cells expressing said transgene;
c. identifying each of said single cells by sequencing the transcriptome of each of the single cells; and
d. correlating the barcode in the transcriptome to a candidate regulatory element
thereby identifying a regulatory element that provides selective expression in the cell type.

2. The method of claim 1, wherein the regulatory element selectively increases expression of the transgene in the cell type.

3. The method of claim 1, wherein the regulatory element provides selective expression of the transgene that is at least 2 fold, at least 4 fold, at least 6 fold, at least 8 fold, or at least 10 fold greater or less as compared to expression driven by another candidate regulatory and/or a control regulatory element in the same cell type.

4. The method of claim 1, wherein the regulatory element provides selective expression of the transgene that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less as compared to expression driven by another candidate regulatory element and/or control regulatory element in the same cell type.

5. The method of claim 1, wherein the regulatory element provides selective expression of the transgene that is about 1.5 times, 2 times, 3 times, 4 times, 5 times, 6 times, 7 times, 7.5 times, 8 times, 9 times, or 10 times greater or less as compared to expression driven by another candidate regulatory element and/or control regulatory element in the same cell type.

6. The method of claim 1, wherein the regulatory element provides selective expression of the transgene that is at least 2 fold, at least 4 fold, at least 6 fold, at least 8 fold, or at least 10 fold greater or less as compared to expression of the transgene from the same regulatory element in a different cell type.

7. The method of claim 1, wherein the regulatory element provides selective expression of the transgene that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less compared to expression of the transgene from the same regulatory element in a different cell type.

8. The method of claim 1, wherein the regulatory element provides selective expression of the transgene that is about 1.5 times, 2 times, 3 times, 4 times, 5 times, 6 times, 7 times, 7.5 times, 8 times, 9 times, or 10 times greater or less compared to expression of the transgene from the same regulatory element in a different cell type.

9. The method of claim 1, wherein the regulatory element provides selective expression of the transgene in one cell type over at least one other cell type.

10. The method of claim 1, wherein the regulatory element provides selective expression of the transgene in parvalbumin (PV) neurons as compared to non-PV neurons.

11. The method of claim 10, wherein the non-PV neuron is one or more of excitatory neurons, dopaminergic neurons, astrocytes, microglia, or motor neurons.

12. A method of identifying a regulatory element that provides selective expression of the transgene in a cell type, comprising:
a. providing cells with a mixture of vectors each comprising a candidate regulatory element operably linked to a transgene, wherein each vector further comprises a barcode;
b. isolating RNA from a plurality of single cells expressing said transgene;
c. identifying each of said single cells by sequencing the transcriptome of each of the single cells;
d. correlating the barcode in the transcriptome to the candidate regulatory element; and
e. comparing expression level of the transgene provided by each candidate regulatory element to a reference expression level of the transgene;
thereby identifying the candidate regulatory element that provides selective expression of the transgene in the cell type.

13. The method of claim 12, wherein the regulatory element selectively increases or decreases expression of the transgene in the cell type.

14. The method of claim 12, wherein the reference expression level of the transgene is provided by a control regulatory element.

15. The method of claim 12, wherein the regulatory element provides selective expression of the transgene that is at least 2 fold, at least 4 fold, at least 6 fold, at least 8 fold, or at least 10 fold greater or less as compared to expression driven by another candidate regulatory element and/or control regulatory element in the same cell type.

16. The method of claim 12, wherein the regulatory element provides selective expression of the transgene that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less as compared to expression driven by another candidate regulatory element and/or control regulatory element in the same cell type.

17. The method of claim 12, wherein the regulatory element provides selective expression of the transgene that is about 1.5 times, 2 times, 3 times, 4 times, 5 times, 6 times, 7 times, 7.5 times, 8 times, 9 times, or 10 times greater or less as compared to expression driven by another candidate regulatory element and/or control regulatory element in the same cell type.

18. The method of claim 12, wherein the reference expression level of the transgene is provided by a pan-cellular regulatory element.

19. The method of claim 12, wherein the pan-cellular regulatory element is selected from the group consisting of cytomegalovirus major immediate-early promoter (CMV), chicken β-actin promoter (CBA), CMV early enhancer/CBA promoter (CAG), elongation factor-1α promoter (EF1α), simian virus 40 promoter (SV40), phosphoglycerate kinase promoter (PGK), and the polyubiquitin C gene promoter (UBC).

20. The method of claim 12, wherein the regulatory element provides selective expression of the transgene that is at least 2 fold, at least 4 fold, at least 6 fold, at least 8 fold, or at least 10 fold greater or less as compared to expression driven by a pan-cellular regulatory element in the same cell type.

21. The method of claim 12, wherein the regulatory element provides selective expression of the transgene that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less as compared to expression driven by a pan-cellular regulatory element in the same cell type.

22. The method of claim 12, wherein the regulatory element provides selective expression of the transgene that is about 1.5 times, 2 times, 3 times, 4 times, 5 times, 6 times, 7 times, 7.5 times, 8 times, 9 times, or 10 times greater or less as compared to expression driven by a pan-cellular regulatory element in the same cell type.

23. The method of claim 12, wherein the regulatory element provides selective expression of the transgene in one cell type over at least one other cell type.

24. The method of claim 12, wherein the regulatory element results in selective expression of the transgene in PV neurons as compared to non-PV neurons.

25. The method of claim 24, wherein the non-PV neuron is one or more of excitatory neurons, dopaminergic neurons, astrocytes, microglia, or motor neurons.

26. A method of identifying a cell type that selectively expresses a transgene operably linked to a regulatory element, comprising:
a. providing cells with a mixture of vectors each comprising a candidate regulatory element operably linked to a transgene, wherein each vector further comprises a barcode;
b. isolating RNA from a plurality of single cells expressing said transgene;
c. identifying each of said single cells by sequencing the transcriptome of each of the single cells;
d. correlating the barcode in the transcriptome to the candidate regulatory element; and
e. comparing expression level of the transgene provided by the candidate regulatory element in one cell type to expression level of the same candidate regulatory element in a different cell type
thereby identifying the cell type that selectively expresses the transgene operably linked regulatory element.

27. The method of claim 26, wherein the regulatory element selectively increases or decreases expression of the transgene in one cell type as compared to at least one other cell type.

28. The method of claim 26, wherein the regulatory element provides selective expression of the transgene in one cell type that is at least 2 fold, at least 4 fold, at least 6 fold, at least 8 fold, or at least 10 fold greater or less as compared to expression driven by the regulatory element in at least one other cell type.

29. The method of claim 26, wherein the regulatory element provides selective expression of the transgene in one cell type that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater or less as compared to expression driven by the regulatory element in at least one other cell type.

30. The method of claim 26, wherein the regulatory element provides selective expression of the transgene in one cell type that is about 1.5 times, 2 times, 3 times, 4 times, 5 times, 6 times, 7 times, 7.5 times, 8 times, 9 times, or 10 times greater or less as compared to expression driven by the regulatory element in at least one other cell type.

31. The method of claim 26, wherein the regulatory element results in selective expression of the transgene in PV neurons as compared to non-PV neurons.

32. The method of claim 31, wherein the non-PV neuron is one or more of excitatory neurons, dopaminergic neurons, astrocytes, microglia, or motor neurons.

33. The method of any one of claims 1-32, wherein the RNA is selected from the group consisting of: mRNA, long noncoding RNA, antisense transcripts, and pri-miRNAs.

34. The method of any one of claims 1-33, wherein the vector is selected from the group consisting of: a plasmid, a viral vector, or a cosmid.

35. The method of claim 34, wherein the viral vector is an adeno-associated virus (AAV) vector.

36. The method of claim 35, wherein the AAV vector is AAV1, AAV8, AAV9, scAAV1, scAAV8, or scAAV9.

37. The method of claim 36, wherein the AAV vector is AAV9.

38. The method of any one of claims 35-37, wherein the vector comprises a 5′ AAV inverted terminal repeat (ITR) sequence and a 3′ AAV ITR sequence.

39. The method of any one of claims 1-38, wherein the mixture of vectors comprises at least 10⁴ candidate regulatory elements.

40. The method of any one of claims 1-39, wherein each candidate regulatory element correlates to at least one unique barcode.

41. The method of any one of claims 1-40, wherein the transgene comprises a reporter gene sequence.

42. The method of claim 41, wherein the reporter gene sequence is operably linked to a sequence encoding a nuclear binding domain.

43. The method of any one of claims 1-43, wherein the transgene comprises the barcode.

44. The method of any one of claims 42-44, wherein the reporter gene sequence comprises the barcode.

45. The method of claim 43 or 44, wherein the barcode comprises alternative codons.

46. The method of any one of claims 43-45, wherein the sequence encoding the nuclear binding domain comprises the barcode.

47. The method of any one of claims 43-46, wherein the sequence encoding the nuclear binding domain encodes a Klarsicht/ANC-1/Syne homology (KASH) domain or a Sad1p/UNC-84 (SUN) domain protein, or biologically active fragment thereof.

48. The method of any one of claims 1-47, wherein the cell type belongs to a tissue selected from the group consisting of: connective tissue, muscular tissue, nervous tissue, and epithelial tissue.

49. A nucleic acid molecule comprising a regulatory element operably linked to a transgene, wherein the nucleic acid molecule comprises a barcode.

50. The nucleic acid molecule of claim 49, wherein the barcode comprises alternative codons.

51. The nucleic acid molecule of claim 49 or 50, wherein the transgene comprises a reporter gene sequence.

52. The nucleic acid molecule of claim 51, wherein the reporter gene sequence is operably linked to a nucleotide sequence encoding a nuclear binding domain sequence.

53. The nucleic acid molecule of claim 52, wherein the nuclear binding domain sequence encodes a KASH domain or SUN domain protein or biologically active fragment thereof.

54. The nucleic acid molecule of any one of claims 50-53, wherein the regulatory element is non-naturally occurring.

55. The nucleic acid molecule of any one of claims 49-54, wherein the reporter gene sequence encodes a fluorescent protein.

56. The nucleic acid molecule of claim 55, wherein the fluorescent protein is a green fluorescent protein (GFP), enhanced green fluorescent protein (EGFP), a yellow fluorescent protein (YFP), such as mBanana, a red fluorescent protein (RFP), such as mCherry, DsRed, dTomato, tdTomato, mHoneydew, or mStrawberry, TagRFP, far-red fluorescent pamidronate (FRFP), such as mGrape1 or mGrape2, a cyan fluorescent protein (CFP), a blue fluorescent protein (BFP), enhanced cyan fluorescent protein (ECFP), ultramarine fluorescent protein (UMFP), orange fluorescent protein (OFP), such as mOrange or mTangerine, red (orange) fluorescent protein (mROFP), TagCFP, or a tetracystein fluorescent motif.

57. The nucleic acid molecule of any one of claims 49-56, wherein the transgene comprises the barcode.

58. The nucleic acid molecule of any one of claims 49-56, wherein the sequence encoding the nuclear binding domain comprises the barcode.

59. The nucleic acid molecule of any one of claims 49-56, wherein the reporter gene sequence comprises the barcode.

60. The nucleic acid molecule of any one of claims 49-59, wherein the barcode is placed within a coding region of the transgene.

61. The nucleic acid molecule of any one of claims 49-59, wherein the nucleic acid molecule comprises a non-coding region, and wherein the barcode is placed within a non-coding region of the transgene.

62. The nucleic acid molecule of claim 61, wherein the nucleic acid molecule comprises an untranslated region (UTR) and the barcode is placed within the UTR.

63. The nucleic acid molecule of claim 61, wherein the nucleic acid comprises a polyA sequence, and wherein the barcode is placed at least 50 bases upstream of the polyA sequence.

64. The nucleic acid molecule of any one of claims 49-59, wherein the barcode is placed upstream of a transcription start site.

65. A nucleic acid molecule, wherein the nucleic acid molecule is an RNA molecule transcribed from a DNA molecule, wherein the RNA molecule comprises a transgene and a barcode sequence, wherein the DNA molecule comprises a regulatory element, and wherein the barcode sequence in the RNA molecule correlates with the regulatory element in the DNA molecule.

66. The nucleic acid molecule of claim 65, wherein the transgene comprises a reporter gene sequence.

67. The nucleic acid molecule of claim 66, wherein the reporter gene sequence is operably linked to a nucleotide sequence encoding a nuclear binding domain.

68. The nucleic acid molecule of claim 67, wherein the nuclear binding domain is a KASH domain or SUN domain protein or biologically active fragment thereof.

69. The nucleic acid molecule of any one of claims 65-68, wherein the regulatory element is non-naturally occurring.

70. The nucleic acid molecule of any one of claims 66-69, wherein the reporter gene sequence encodes a fluorescent protein.

71. The nucleic acid molecule of claim 70, wherein the fluorescent protein is a green fluorescent protein (GFP), enhanced green fluorescent protein (EGFP), a yellow fluorescent protein (YFP), such as mBanana, a red fluorescent protein (RFP), such as mCherry, DsRed, dTomato, tdTomato, mHoneydew, or mStrawberry, TagRFP, far-red fluorescent pamidronate (FRFP), such as mGrape1 or mGrape2, a cyan fluorescent protein (CFP), a blue fluorescent protein (BFP), enhanced cyan fluorescent protein (ECFP), ultramarine fluorescent protein (UMFP), orange fluorescent protein (OFP), such as mOrange or mTangerine, red (orange) fluorescent protein (mROFP), TagCFP, or a tetracystein fluorescent motif.

72. The nucleic acid molecule of any one of claims 65-71, wherein the transgene comprises the barcode.

73. The nucleic acid molecule of any one of claims 67-71, wherein the sequence encoding the nuclear binding domain comprises the barcode.

74. The nucleic acid molecule of any one of claims 66-71, wherein the reporter gene sequence comprises the barcode.

75. The nucleic acid molecule of any one of claims 66-74, wherein the barcode comprises alternative codons.

76. The nucleic acid molecule of any one of claims 65-71, wherein the nucleic acid molecule comprises an untranslated region (UTR) and the barcode is placed within the UTR.

77. The nucleic acid molecule of any one of claims 65-71, wherein the nucleic acid molecule comprises a polyA sequence, and wherein the barcode is placed at least 50 bases upstream of the polyA sequence.

78. The nucleic acid molecule of any one of claims 65-71, wherein the barcode is placed upstream of a transcription start site.

79. The nucleic acid molecule of any one of claims 65-77, wherein the nucleic acid molecule is connected to a microparticle.

80. The nucleic acid molecule of claim 79, wherein the microparticle is a bead.

81. The nucleic acid molecule of claim 79 or 80, wherein the microparticle is connected to a microparticle polynucleotide molecule.

82. The nucleic acid molecule of claim 81, wherein the nucleic acid molecule is connected to the microparticle via the microparticle polynucleotide molecule.

83. The nucleic acid molecule of claim 81 or 82, wherein the microparticle polynucleotide molecule comprises a primer sequence.

84. The nucleic acid molecule of any one of claims 81-83, wherein the microparticle polynucleotide molecule comprises a cell barcode sequence.

85. The nucleic acid molecule of any one of claims 81-84, wherein the microparticle polynucleotide molecule comprises a Unique Molecular Identifier (UMI) nucleotide sequence.

86. The nucleic acid molecule of any one of claims 81-85, wherein the microparticle polynucleotide molecule comprises an oligo-dT sequence.

87. The nucleic acid molecule of any one of claims 81-86, wherein the microparticle polynucleotide molecule comprises: a) a primer sequence, b) a cell barcode sequence, c) a Unique Molecular Identifier (UMI) nucleotide sequence, and d) an oligo-dT sequence; wherein the nucleic acid comprises a polyA nucleotide sequence, and wherein the microparticle is connected to a)-d) in the following order: microparticle--a)--b)--c)--d); and wherein the polyA nucleotide sequence is hybridized with the oligo-dT sequence.

88. The nucleic acid molecule of claim 87, wherein the microparticle is a bead.

89. A vector comprising the nucleic acid of any one of claims 49-65.

90. The vector of claim 89, wherein the vector is a viral vector.

91. The vector of claim 89, wherein the vector is an adeno-associated viral vector.

92. The vector of claim 89, wherein the adeno-associated viral vector is any one of AAV1, AAV2, AAV3, AAV4, AAV5, AAV6, AAV7, AAV8, AAV9, AAV10, AAV 11, AAV12, rh10, and hybrids thereof, avian AAV, bovine AAV, canine AAV, equine AAV, primate AAV, non-primate AAV, or ovine AAV.

93. The vector of claim 89, wherein the adeno-associated viral vector is an AAV9 vector.

94. A cell comprising the nucleic acid of any one of claims 49-87.

95. A cell comprising the vector of any one of claims 88-92.

96. A microparticle connected to one or more of the nucleic acid of any one of claims 65-88.

97. The microparticle of claim 96, wherein the microparticle is a bead.

98. The microparticle of claim 96 or 97, wherein the microparticle is connected to a microparticle polynucleotide molecule.

99. The microparticle of claim 98, wherein the microparticle polynucleotide molecule comprises a primer sequence.

100. The microparticle of claim 98 or 99, wherein the microparticle polynucleotide molecule comprises a Unique Molecular Identifier (UMI).

101. The microparticle of any one of claims 98-100, wherein the microparticle polynucleotide molecule comprises an oligo-dT sequence.

102. The microparticle of claim 98-101, wherein the nucleic acid comprises a polyA nucleotide sequence, and wherein the polyA nucleotide sequence is hybridized to the oligo-dT sequence.

103. The microparticle of any one of claims 98-102, wherein the microparticle polynucleotide molecule comprises: a) a primer sequence, b) a cell barcode sequence, c) a Unique Molecular Identifier (UMI) sequence, and d) an oligo-dT sequence; wherein the nucleic acid comprises a polyA nucleotide sequence, wherein the microparticle is connected to a)-d) in the following order: microparticle--a)--b)--c)--d); and wherein the polyA nucleotide sequence is hybridized with the oligo-dT sequence.

104. The microparticle of claim 103, wherein the microparticle is a bead.

105. A droplet comprising the nucleic acid molecule of any one of claims 49-88.

106. A droplet comprising the cell of claim 94 or 95.

107. A droplet comprising the microparticle of any one of claims 96-104.

108. A droplet comprising the cell of claim 94 or 95 and the microparticle of any one of claims 96-104.


SEQUENCE LISTING (70 sequences)

SEQ ID NO: 1 - DNA, 1878 bp, Artificial Sequence
  Description of Artificial Sequence Synthetic polynucleotide
tcaacagggggacacttgggaaagaaggatggggacagagccgagaggactgttacacattagagaaacatcagtgactgtgccagctttggggtagactgcacaaaagccctgaggcagcacaggcaggatccagtctgctggtcccaggaagctaaccgtctcagacagagcacaaagcaccgagacatgtgccacaaggcttgtgtagagaggtcagaggacagcgtacaggtcccagagatcaaactcaacctcaccaggcttggcagcaagcctttaccaacccacccccaccccacccaccctgcacgcgcccctctcccctccccatggtctcccatggctatctcacttggccctaaaatgtttaaggatgacactggctgctgagtggaaatgagacagcagaagtcaacagtagattttaggaaagccagagaaaaaggcttgtgctgtttttagaaagccaagggacaagctaagatagggcccaagtaatgctagtatttacatttatccacacaaaacggacgggcctccgctgaaccagtgaggccccagacgtgcgcataaataacccctgcgtgctgcaccacctggggagagggggaggaccacggtaaatggagcgagcgcatagcaaaagggacgcggggtccttttctctgccggtggcactgggtagctgtggccaggtgtggtactttgatggggcccagggctggagctcaaggaagcgtcgcagggtcacagatctgggggaaccccggggaaaagcactgaggcaaaaccgccgctcgtctcctacaatatatgggagggggaggttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccccagcagccgaaggcgctactaggaacggtaacctgttacttttccaggggccgtagtcgacccgctgcccgagttgctgtgcgactgcgcgcgcggggctagagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccgctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctg

SEQ ID NO: 2 - DNA, 258 bp, Artificial Sequence
  Description of Artificial Sequence Synthetic polynucleotide
gaggaggaggaggagacagacagcaggatgccccacctcgacagccccggttcatcacaaccgagacgctccttcctctcaagggtgatcagggcagctctaccgttgcagctgcttctgctgctgctgctgctcctggcctgcctgttgcctgcttcagaggatgactacagctgcacccaggccaacaactttgcccgatccttctaccccatgctgcggtacaccaacgggccacctcccacctaggactcagct

SEQ ID NO: 3 - DNA, 258 bp, Artificial Sequence
  Description of Artificial Sequence Synthetic polynucleotide
gaggaggaggaggagacagacagcaggatgccccacctcgacagccccggtagcagccaaccgagacgctccttcctctcaagggtgatcagggcagctctaccgttgcagctgcttctgctgctgctgctgctcctggcctgcctgttgcctgccagcgaggatgactacagctgcacccaggccaacaactttgcccgatccttctaccccatgctgcggtacaccaacgggccacctcccacctagcttactagc

SEQ ID NO: 4 - DNA, 258 bp, Artificial Sequence
  Description of Artificial Sequence Synthetic polynucleotide
gaggaggaggaggagacagacagcaggatgccccacctcgacagccccggcagtagtcaaccgagacgctccttcctctcaagggtgatcagggcagctctaccgttgcagctgcttctgctgctgctgctgctcctggcctgcctgttgcccgctagtgaggatgactacagctgcacccaggccaacaactttgcccgatccttctaccccatgctgcggtacaccaacgggccacctcccacctagtcaggaatc

SEQ ID NO: 5 - DNA, 258 bp, Artificial Sequence
  Description of Artificial Sequence Synthetic polynucleotide
gaggaggaggaggagacagacagcaggatgccccacctcgacagccccggctcgtcgcaaccgagacgctccttcctctcaagggtgatcagggcagctctaccgttgcagctgcttctgctgctgctgctgctcctggcctgcctgttgcccgcctcggaggatgactacagctgcacccaggccaacaactttgcccgatccttctaccccatgctgcggtacaccaacgggccacctcccacctagagacaggta

SEQ ID NO: 6 - DNA, 258 bp, Artificial Sequence
  Description of Artificial Sequence Synthetic polynucleotide
gaggaggaggaggagacagacagcaggatgccccacctcgacagccccggatcttctcaaccgagacgctccttcctctcaagggtgatcagggcagctctaccgttgcagctgcttctgctgctgctgctgctcctggcctgcctgttgccagcatctgaggatgactacagctgcacccaggccaacaactttgcccgatccttctaccccatgctgcggtacaccaacgggccacctcccacctaggattctcag

SEQ ID NO: 7 - DNA, 258 bp, Artificial Sequence
  Description of Artificial Sequence Synthetic polynucleotide
gaggaggaggaggagacagacagcaggatgccccacctcgacagccccgggtcctcccaaccgagacgctccttcctctcaagggtgatcagggcagctctaccgttgcagctgcttctgctgctgctgctgctcctggcctgcctgttgccggcgtccgaggatgactacagctgcacccaggccaacaactttgcccgatccttctaccccatgctgcggtacaccaacgggccacctcccacctagcagatacca

SEQ ID NO: 8 - DNA, 9 bp, Artificial Sequence
  Description of Artificial Sequence Synthetic oligonucleotide
cccctggtt

SEQ ID NO: 9 - DNA, 12 bp, Artificial Sequence
  Description of Artificial Sequence Synthetic oligonucleotide
ggttcatcacaa

SEQ ID NO: 10 - DNA, 15 bp, Artificial Sequence
  Description of Artificial Sequence Synthetic oligonucleotide
ttgcctgcttcagag

SEQ ID NO: 11 - DNA, 9 bp, Artificial Sequence
  Description of Artificial Sequence Synthetic oligonucleotide
ctaacggtt

SEQ ID NO: 12 - DNA, 9 bp, Artificial Sequence
  Description of Artificial Sequence Synthetic oligonucleotide
gtggattct

SEQ ID NO: 13 - DNA, 12 bp, Artificial Sequence
  Description of Artificial Sequence Synthetic oligonucleotide
ggtagcagccaa

SEQ ID NO: 14 - DNA, 15 bp, Artificial Sequence
  Description of Artificial Sequence Synthetic oligonucleotide
ttgcctgccagcgag

SEQ ID NO: 15 - DNA, 9 bp, Artificial Sequence
  Description of Artificial Sequence Synthetic oligonucleotide
ctttctctc

SEQ ID NO: 16 - DNA, 9 bp, Artificial Sequence
  Description of Artificial Sequence Synthetic oligonucleotide
ggtggtact

SEQ ID NO: 17 - DNA, 12 bp, Artificial Sequence
  Description of Artificial Sequence Synthetic oligonucleotide
ggcagtagtcaa

SEQ ID NO: 18 - DNA, 15 bp, Artificial Sequence
  Description of Artificial Sequence Synthetic oligonucleotide
ttgcccgctagtgag

SEQ ID NO: 19 - DNA, 9 bp, Artificial Sequence
  Description of Artificial Sequence Synthetic oligonucleotide
tcccatcat

SEQ ID NO: 20 - DNA, 9 bp, Artificial Sequence
  Description of Artificial Sequence Synthetic oligonucleotide
ggttccttc

SEQ ID NO: 21 - DNA, 12 bp, Artificial Sequence
  Description of Artificial Sequence Synthetic oligonucleotide
ggctcgtcgcaa

SEQ ID NO: 22 - DNA, 15 bp, Artificial Sequence
  Description of Artificial Sequence Synthetic oligonucleotide
ttgcccgcctcggag

SEQ ID NO: 23 - DNA, 9 bp, Artificial Sequence
  Description of Artificial Sequence Synthetic oligonucleotide
aagttggcg

SEQ ID NO: 24 - DNA, 9 bp, Artificial Sequence
  Description of Artificial Sequence Synthetic oligonucleotide
ggtggtact

SEQ ID NO: 25 - DNA, 12 bp, Artificial Sequence
  Description of Artificial Sequence Synthetic oligonucleotide
ggatcttctcaa

SEQ ID NO: 26 - DNA, 15 bp, Artificial Sequence
  Description of Artificial Sequence Synthetic oligonucleotide
ttgccagcatctgag

SEQ ID NO: 27 - DNA, 9 bp, Artificial Sequence
  Description of Artificial Sequence Synthetic oligonucleotide
tcccatcat

SEQ ID NO: 28 - DNA, 9 bp, Artificial Sequence
  Description of Artificial Sequence Synthetic oligonucleotide
ggaggcaag

SEQ ID NO: 29 - DNA, 12 bp, Artificial Sequence
  Description of Artificial Sequence Synthetic oligonucleotide
gggtcctcccaa

SEQ ID NO: 30 - DNA, 15 bp, Artificial Sequence
  Description of Artificial Sequence Synthetic oligonucleotide
ttgccggcgtccgag

SEQ ID NO: 31 - DNA, 9 bp, Artificial Sequence
  Description of Artificial Sequence Synthetic oligonucleotide
catcaatcg

SEQ ID NO: 32 - DNA, 9 bp, Artificial Sequence
  Description of Artificial Sequence Synthetic oligonucleotide
tcgcaatct

SEQ ID NO: 33 - DNA, 12 bp, Artificial Sequence
  Description of Artificial Sequence Synthetic oligonucleotide
ggttcgtcgcag

SEQ ID NO: 34 - DNA, 15 bp, Artificial Sequence
  Description of Artificial Sequence Synthetic oligonucleotide
ctccctgcatcggaa

SEQ ID NO: 35 - DNA, 9 bp, Artificial Sequence
  Description of Artificial Sequence Synthetic oligonucleotide
acggctaca

SEQ ID NO: 36 - DNA, 9 bp, Artificial Sequence
  Description of Artificial Sequence Synthetic oligonucleotide
cgctaccag

SEQ ID NO: 37 - DNA, 12 bp, Artificial Sequence
  Description of Artificial Sequence Synthetic oligonucleotide
ggttcttctcag

SEQ ID NO: 38 - DNA, 15 bp, Artificial Sequence
  Description of Artificial Sequence Synthetic oligonucleotide
ctccctgcttctgaa

SEQ ID NO: 39 - DNA, 9 bp, Artificial Sequence
  Description of Artificial Sequence Synthetic oligonucleotide
gcgtcgtaa

SEQ ID NO: 40 - DNA, 9 bp, Artificial Sequence
  Description of Artificial Sequence Synthetic oligonucleotide
acaacacct

SEQ ID NO: 41 - DNA, 12 bp, Artificial Sequence
  Description of Artificial Sequence Synthetic oligonucleotide
ggctcctcccag

SEQ ID NO: 42 - DNA, 15 bp, Artificial Sequence
  Description of Artificial Sequence Synthetic oligonucleotide
ctccccgcatccgaa

SEQ ID NO: 43 - DNA, 9 bp, Artificial Sequence
  Description of Artificial Sequence Synthetic oligonucleotide
atgacgacc

SEQ ID NO: 44 - DNA, 9 bp, Artificial Sequence
  Description of Artificial Sequence Synthetic oligonucleotide
aaagtcccg

SEQ ID NO: 45 - DNA, 12 bp, Artificial Sequence
  Description of Artificial Sequence Synthetic oligonucleotide
ggctcatcacag

SEQ ID NO: 46 - DNA, 15 bp, Artificial Sequence
  Description of Artificial Sequence Synthetic oligonucleotide
ctccccgcgtcagaa

SEQ ID NO: 47 - DNA, 9 bp, Artificial Sequence
  Description of Artificial Sequence Synthetic oligonucleotide
tctcatccg

SEQ ID NO: 48 - DNA, 9 bp, Artificial Sequence
  Description of Artificial Sequence Synthetic oligonucleotide
gacttctct

SEQ ID NO: 49 - DNA, 12 bp, Artificial Sequence
  Description of Artificial Sequence Synthetic oligonucleotide
ggaagcagccag

SEQ ID NO: 50 - DNA, 15 bp, Artificial Sequence
  Description of Artificial Sequence Synthetic oligonucleotide
ctcccagccagcgaa

SEQ ID NO: 51 - DNA, 9 bp, Artificial Sequence
  Description of Artificial Sequence Synthetic oligonucleotide
tccacggtt

SEQ ID NO: 52 - DNA, 9 bp, Artificial Sequence
  Description of Artificial Sequence Synthetic oligonucleotide
actccaact

SEQ ID NO: 53 - DNA, 12 bp, Artificial Sequence
  Description of Artificial Sequence Synthetic oligonucleotide
gggagtagtcag

SEQ ID NO: 54 - DNA, 15 bp, Artificial Sequence
  Description of Artificial Sequence Synthetic oligonucleotide
ctcccggccagtgaa

SEQ ID NO: 55 - DNA, 9 bp, Artificial Sequence
  Description of Artificial Sequence Synthetic oligonucleotide
ttccagctc

SEQ ID NO: 56 - DNA, 9 bp, Artificial Sequence
  Description of Artificial Sequence Synthetic oligonucleotide
caggctgaa

SEQ ID NO: 57 - DNA, 12 bp, Artificial Sequence
  Description of Artificial Sequence Synthetic oligonucleotide
ggtagttctcag

SEQ ID NO: 58 - DNA, 15 bp, Artificial Sequence
  Description of Artificial Sequence Synthetic oligonucleotide
ttgcctgcatctgaa

SEQ ID NO: 59 - DNA, 9 bp, Artificial Sequence
  Description of Artificial Sequence Synthetic oligonucleotide
ttcgcattg

SEQ ID NO: 60 - DNA, 9 bp, Artificial Sequence
  Description of Artificial Sequence Synthetic oligonucleotide
cgtcgatgc

SEQ ID NO: 61 - DNA, 12 bp, Artificial Sequence
  Description of Artificial Sequence Synthetic oligonucleotide
ggcagctcccaa

SEQ ID NO: 62 - DNA, 15 bp, Artificial Sequence
  Description of Artificial Sequence Synthetic oligonucleotide
ttgccagctagcgag

SEQ ID NO: 63 - DNA, 9 bp, Artificial Sequence
  Description of Artificial Sequence Synthetic oligonucleotide
gactccact

SEQ ID NO: 64 - DNA, 9 bp, Artificial Sequence
  Description of Artificial Sequence Synthetic oligonucleotide
gttcggaaa

SEQ ID NO: 65 - DNA, 12 bp, Artificial Sequence
  Description of Artificial Sequence Synthetic oligonucleotide
gggagctcccag

SEQ ID NO: 66 - DNA, 15 bp, Artificial Sequence
  Description of Artificial Sequence Synthetic oligonucleotide
ttgccggcaagtgag

SEQ ID NO: 67 - DNA, 9 bp, Artificial Sequence
  Description of Artificial Sequence Synthetic oligonucleotide
actccgtcg

SEQ ID NO: 68 - DNA, 70 bp, Artificial Sequence
  Description of Artificial Sequence Synthetic primer
aatgatacggcgaccaccgagatctacactagatcgcacactctttccctacacgacgctcttccgatct

SEQ ID NO: 69 - DNA, 57 bp, Artificial Sequence
  Description of Artificial Sequence Synthetic primer
gtgactggagttcagacgtgtgctcttccgatctgatccttctaccccatgctgcgg

SEQ ID NO: 70 - DNA, 65 bp, Artificial Sequence
  Description of Artificial Sequence Synthetic primer
  modified_base (25)..(32): a, c, t, g, unknown or other
caagcagaagacggcatacgagatnnnnnnnngtgactggagttcagacgtgtgctcttccgatc